(19) United States
(12) Patent Application Publication
(10) Pub. No.: US 2022/0308022 A1
(43) Pub. Date: Sep. 29, 2022

(54) CHARACTERIZATION OF GENE THERAPY VIRAL PARTICLES USING SIZE EXCLUSION CHROMATOGRAPHY AND MULTI-ANGLE LIGHT SCATTERING TECHNOLOGIES

(71) Applicant: BIOMARIN PHARMACEUTICAL INC., Novato, CA (US)

(72) Inventors: Vikas BHAT, Novato, CA (US); Geoffrey Yehuda BERGUIG, Novato, CA (US); Nicole Louise MCINTOSH, Novato, CA (US)

(73) Assignee: BIOMARIN PHARMACEUTICAL INC., Novato, CA (US)

(21) Appl. No.: 17/635,488

(22) PCT Filed: Sep. 25, 2020
(86) PCT No.: PCT/US2020/052738

§ 371 Date: Feb. 15, 2022

Related U.S. Application Data
(60) Provisional application No. 62/907,509, filed on Sep. 27, 2019.
(60) Provisional application No. 63/043,571, filed on Jun. 24, 2020.

(51) Int. Cl. G01N 30/74; C12N 7/00; G01N 15/02

(52) U.S. Cl. CPC G01N 30/74; C12N 7/00; G01N 15/0205; C12N 2750/14122; C12N 2750/14123; C12N 2750/14151; C12N 2740/15022; C12N 2740/15023; C12N 2740/15051; G01N 2030/027

(57) ABSTRACT

This disclosure relates to the use of size exclusion chromatography and/or size exclusion chromatography with multi-angle light scattering technology to characterize viral particles such as adeno-associated virus and lentivirus particles. The disclosed methods are also useful for estimating the titer of viral particles, determining the integrity of the viral particles and estimating the amount of DNA encapsidated in the viral particle.

### CROSS-REFERENCE TO RELATED APPLICATIONS

This application claims the benefit of U.S. Provisional Application Ser. No. 62/907,509, filed on Sep. 27, 2019, and U.S. Provisional Application Ser. No. 63/043,571, filed on Jun. 24, 2020, the entirety of which are incorporated by reference herein.

### FIELD

This disclosure relates to the use of size exclusion chromatography and/or size exclusion chromatography with multi-angle light scattering technology to characterize viral particles such as adeno-associated virus and lentivirus particles. The disclosed methods are also useful for estimating the titer of viral particles, determining the integrity of the viral particles and estimating the amount of deoxyribonucleic acid encapsidated in the viral particle.

### BACKGROUND

Adeno-associated virus (AAV) is a small, replication-defective, non-enveloped animal virus that infects humans and some other primate species. Several features of AAV make this virus an attractive vehicle for delivery of therapeutic proteins by gene therapy, including, for example, that AAV is not known to cause human disease and induces a mild immune response, and that AAV vectors can infect both dividing and quiescent cells without integrating into the host cell genome.

AAVs consist of a family of virus particles in size range of 25 nm from the Parvoviridae family. These are non-enveloped viruses with icosahedral protein shell made of three different viral proteins (VP1, VP2 and VP3) encapsidating a single stranded DNA genome of ˜4.7 kilobases (kb) in length (Balakrishnan et al., Curr Gene Ther 14, 86-100, 2014). Due to their relative safety and long-term gene expression, different serotypes of recombinant AAV vectors are currently used in various gene therapy programs, both at non-clinical and clinical stage. At present 150 gene therapy trials using recombinant AAVs (rAAVs) are ongoing worldwide in Phase I, II and III stages, of them are in Phase III clinical trials and 6 of those are running in USA (http://www.abedia.com/wiley/search.php). Several purification methods have been developed to generate these virus particles at large scale for use in clinic over the years with striking success (Brument et al., Mol Ther 6, 678-686, 2002, Qu et al., J Virol Methods 140, 183-192, 2007, Okada et al., Hum Gene Ther 20, 1013-1021, 2009, Mietzsch et al., Hum Gene Ther 25, 212-222, 2014, Clement et al., Mol Ther Methods Clin Dev 3, 16002, 2016). However, development of robust analytics to account for all variables that exist in overall process development and final purified AAV drug product is still a developing field.

Recombinant lentiviruses (rLVs) are useful for delivering heterologous transgenes (i.e., genes that are not native to the lentivirus (LV)) to hematopoietic stem cells in order to treat genetic diseases such as adenosine deaminase deficiency (Farinelli, et al, 2014), β-thalassemia, sickle cell disease (Negre et al., 2016), severe combined immune deficiencies, metachromatic leukodystrophy, adrenoleukodystrophy, Wiskott-Aldrich syndrome, chronic granulomatous disease (Booth et al., 2016), and several lysosomal storage disorders (Rastall, et al., 2015).

The LV genus belongs to the Retroviridae family. Characterized by a long incubation period before disease onset in a host, LV received its name from the latin “lente”, meaning “slow” (Milone and O′Doherty, Leukemia 32,1529-1541, 2018). Through extensive optimization of lentiviral capsids, the successful production of nonpathogenic lentiviral vectors, which are not capable of replication after initial gene delivery, has established the safety and efficiency of LV vector biotherapy. Most of these vectors are based on human immunodeficiency virus (HIV), which consists of two single-stranded RNA copies coated by 2,000 p24 proteins arranged into a conical capsid (Milone and O'Doherty, Leukemia 32,1529-1541, 2018). Capsid integrity is further protected by a p17 matrix, all of which is enveloped by a roughly spherical —120 nm diameter phospholipid envelope (Milone and O'Doherty, Leukemia 32,1529-1541, 2018). The large Megadalton size-range of these viral particles enables them to encapsulate a large genome up to 8.5 kb (Milone and O'Doherty, Leukemia 32,1529-1541, 2018)³¹. This single-stranded RNA genome codes for nine genes, five indispensable for viral survival and function and four accessory genes, flanked by long terminal repeats (LTRs) (Milone and O'Doherty, Leukemia 32,1529-1541, 2018). In recombinant LV vectors designed for gene therapy purposes, the phospholipid envelope is replaced most often by a vesicular stomatitis virus G (VSV-G) envelope, which recognizes the ubiquitously-expressed lipoprotein (LDL) receptor, thus enabling vector transduction in a diverse range of cell types (Milone and O'Doherty, Leukemia 32,1529-1541, 2018). Additionally, viral accessory genes are removed to enhance vector safety and are replaced by a therapeutic gene expression cassette. The general infection process involves cellular entry by receptor-mediated endocytosis or membrane fusion following attachment of glycoproteins on the vector envelope with their respective cell-membrane receptors (Milone and O'Doherty, Leukemia 32,1529-1541, 2018). Cellular entry is followed by genome-uncoating and reverse transcription of the released single stranded ribonucleic acid (ssRNA). The newly synthesized cDNA is then transported to the nucleus where it integrates with the host genome. Vital to enhancing the rational design of LV vectors is the detailed understanding of the steps involving this infectivity. However, many aspects of these steps are not fully understood.

One area of particular importance is the genome-uncoating step of the LV infectivity process. Importantly, reverse transcription of the delivered ribonucleic acid (RNA) only takes place upon successful genome uncoating (Milone and O'Doherty, Leukemia 32,1529-1541, 2018). Therefore, it can be reasoned that the cellular transduction efficiency of LV vectors is, in part, dependent on their uncoating efficiency. To date, little is known for sure about LV genome-uncoating. However, it has been speculated that local changes which lower pH promote the uncoating process (Milone and O'Doherty, Leukemia 32,1529-1541, 2018). Furthermore, limited literature is available regarding the use of biophysical tools and characterization methods to evaluate the relationship between LV biological and physical properties.

Gene therapy programs have shown linearity between the presences of gene copy numbers and protein expression as well as potential adverse effects.8-10 A successful gene therapy program therefore is highly dependent on the safety and efficacy outcome post dosage which in turn is dependent on precise and accurate method of AAV vector titration and characterization. Different methods like electron microscopy, dynamic light scattering, analytical ultracentrifugation (AUC), enzyme-linked immunosorbent assay (ELISA), and polymerase chain reaction (PCR) have been separately employed to keep a track of different attributes of purified capsid particles including size, aggregation propensity, stability, amount of empty to full capsids, capsid and vector genome titer respectively. Although they have been used for a while in field of gene therapy, each existing method has its own associated limitations ranging from low throughput to high variability.

Bulk optical density measurement has been tested in past to quantify vector genome and capsid protein content of adenovirus preparations (Maizel et al. Virology 36, 115-125, 1968, Mittereder et al., J Virol 70, 7498-7509, 1996, Sweeney et al., Virology 295, 284-288., 2002). Later, similar idea was applied to quantify the vector genome and capsid protein content of relatively simple structured AAV2 capsid particles (Sommer et al., Mol Ther 7, 122-128, 2003). Although, the results showed good similarity with the existing methods like qPCR and ELISA upon comparison, one major limitation was the significant effect of extrinsic impurities in the solution like protein, nucleic acids and buffer components that can absorb in the same UV range. Moreover, since the method is a bulk measurement of the capsid solution, estimation of the amount of extrinsic impurities as well as capsid aggregates separately was not possible. Due to these potential limitations and the fact that the method couldn't specifically quantify the encapsidated genome within the capsids, the usage of this method has been restricted to academic labs and non-clinical trials.

There is a need for more efficient high throughput techniques for characterizing gene therapy viral particles. For example, there is a need for accurate methods capable of counting viral particles and determining the size distribution of viruses in samples with unknown or hard-to-determine viral concentrations. The methods disclosed herein are highly reliable and allow for rapid and automated size distribution analysis and quantification of viral particles.

### SUMMARY

Provided herein are high throughput and multipurpose characterization methods that utilizes separation ability of size exclusion chromatography (SEC) or SEC in combination with multiangle light scattering (MALS) technology to monitor viral particle capsid size, presence of higher order capsid aggregates, non-encapsidated deoxyribonucleic acid (DNA) and protein impurities, capsid integrity, amount of light capsids, molecular mass of capsid protein and encapsidated DNA, capsid titer and genome titer. The methods utilize capsid protein and encapsidated DNA absorbance properties at ultraviolet (UV) 280 nanometer (nm) and UV 260 nm respectively coupled refractometer and light scattering to monitor multiple attributes of AAV capsids in a single run. An accurate method capable of counting viral particles and determining the size distribution of viruses in samples with unknown or hard-to-determine viral concentrations is highly desirable. The characterization of such properties in viral samples may provide more information to help shorten the development time associated with optimizing virus preparation conditions and identifying stable formulations. Separating viral particles by their size enables estimation of the amount of aggregated viral particles and yields a more accurate virus count. Once the aggregated viral particles are separated, their aggregation state (number of individual virions per aggregate and geometry of the aggregate) can be characterized.

The present disclosure provides methods, processes, and systems for characterizing and quantifying viral particles such as AAV and LV particles using SEC and/or size exclusion chromatography with multi-angle light scattering (SEC-MALS).

SEC, a size based separation technique has the advantage of separating higher order aggregates from monomeric species based on partial exclusion of bigger species from the pores of the stationary phase. The method has been used in case of large VLPs (virus like particle) like influenza particles and uses the UV absorbance properties of these molecules to monitor their elution profile. (Vajda et al. J Chromatogr A 1465, 117-125, 2016, Weigel et al., J Virol Methods 207, 45-53, 2014, Lagoutte et al., J Virol Methods 232, 8-11, 2016, Yang et al., Vaccine 33, 1143-1150, 2015, Ladd Effio et al. Vaccine 34, 1259-1267, 2016). The utility of this technique can be further enhanced if coupled with multiangle light scattering (MALS) and refractive index (RI) detectors. MALS has been widely used in association with SEC or field flow fractionation to determine the absolute molecular weight, size, conformation and distribution of polymers as well as protein bio therapeutics (Ye et al., Anal Biochem 356, 76-85, 2006, Wyatt et al., Analytica Chimica Acta 272, 1-40, 1993, Zinovyev et al., ChemSusChem 11, 3259-3268, 2018, Letourneau et al., Protein Pept Lett 25, 973-979, 2018, Sahin et al., Methods Mol Biol 899, 403-423, 2012 Minton et al., Anal Biochem 501, 4-22, 2016). More recently, some studies have shown that along with other properties light scattering can also be used for quantification of VLPs in solution (Steppert et al., J Chromatogr A 1487, 89-99, 2017, McEvoy et al., Biotechnol Prog 27, 547-554, 2011, Weiet al., J Virol Methods 144, 122-132, 2007, Makra et al., Methods 2, 91-99, 2015).

The development and use of an SEC-MALS characterization method in combination with various biophysical tools to comprehensively characterize LV vector structure and stability as a function of pH is also described. Notably, the described SEC-MALS method is identified as a rapid and reproducible way to directly quantify LV particles without need of a calibration curve and therein estimate LV particle titer. Additionally, in agreement with the proposed genome-uncoating mechanism, our results demonstrate LV capsid destabilization under acidic conditions.

In one aspect, the methods, processes, and systems of various embodiments include the steps of analyzing by SEC a sample of a preparation of viral particles having vector genomes encapsulated within capsids and from the SEC analysis, characterizing and quantifying the viral particles. The characterizing and quantifying include quantifying aggregation of the viral particles in the preparation, quantifying a concentration of nucleic acid and/or protein impurities in the preparation, determining weight averaged molecular weights of the capsids and vector genomes, and quantifying concentrations of viral particles and capsids devoid of encapsulated vector genomes in the preparation. The SEC analysis includes the steps of fractioning the sample by size exclusion chromatography, measuring properties of the viral particles, and characterizing the viral particles. The measurements include measuring light absorption by the fractions at wavelengths of about 260 nm and about 280 nm. In a refinement, the characterizing step includes identifying viral particles such as AAV in at least one of the fractions when the at least one of the fractions exhibits a first wavelength to the second wavelength (first wavelength: second wavelength) absorbance ratio of 1.13 to less than 1.75. In various embodiments, the methods, processes, and systems of various embodiments further include the step of, prior to SEC analysis, analyzing the sample by analytical ultracentrifugation to identify the viral particles and/or capsids without encapsidated vector genomes.

In another aspect, the methods, processes, and systems of various embodiments include the steps of analyzing by SEC and SEC-MALS a sample of a preparation of viral particles having vector genomes encapsulated within capsids and from the SEC and SEC-MALS analysis, characterizing and quantifying the viral particles. The characterizing and quantifying include quantifying aggregation of the viral particles in the preparation, quantifying a concentration of nucleic acid and/or protein impurities in the preparation, determining weight averaged molecular weights of the capsids and vector genomes, and quantifying concentrations of viral particles and capsids devoid of encapsulated vector genomes in the preparation. The SEC analysis includes the steps of fractioning the sample by size exclusion chromatography, measuring properties of the viral particles, and characterizing the viral particles. The measurements include measuring light absorption by the fractions at wavelengths of about 260 nm and about 280 nm. In a refinement, the characterizing step includes identifying viral particles such as AAV in at least one of the fractions when the at least one of the fractions exhibits a first wavelength to the second wavelength (first wavelength: second wavelength) absorbance ratio of 1.13 to less than 1.75. The SEC-MALS analysis includes the steps of fractioning the sample by size exclusion chromatography, measuring properties of the viral particles, and characterizing the viral particles. The measurements including the refractive indexes of the fractions, the light absorption by the fractions at a wavelength within the ultraviolet spectrum, and the intensity of light scatter by the fractions using MALS. In various embodiments, the methods, processes, and systems further include the step of determining a size distribution of the viral particles and/or the capsids devoid of encapsulated vector genomes in the preparation by dynamic light scattering analysis. The size distribution of the viral particles can include the radius of gyration (Rg) and/or hydrodynamic radius (Rh) of the viral particles. In various embodiments, the quantifying concentrations of viral particles and capsids devoid of encapsulated vector genomes in the preparation of various embodiments can include determining the Rg and Rh of the viral particles and/or the capsids devoid of encapsulated vector genomes by dynamic light scattering analysis, wherein a ratio of Rg to Rh correlates to the percentage concentration of viral particles in the preparation. In various embodiments, the methods, processes, and systems of various embodiments further include the step of, prior to SEC or SEC-MALS analysis, analyzing the sample by analytical ultracentrifugation to identify the viral particles and/or capsids without encapsidated vector genomes.

Under another aspect, the methods, processes, and systems of various embodiments include the steps of analyzing by SEC a plurality of samples from a preparation of viral particles having vector genomes encapsulated within capsids and from the SEC analysis, monitoring the structural integrity of the capsids in each of the samples. Each of the samples is modified to have a property that is different from the others. For example, the property is storage of the samples of lengths of time at 25° C. Also, the changes in protein and nucleic acid concentrations between the samples correlate to changes in the structural integrity of the capsids. The SEC analysis includes the steps of fractioning the sample by size exclusion chromatography, measuring properties of the viral particles, and characterizing the viral particles. The measurements include measuring light absorption by the fractions at wavelengths of about 260 nm and about 280 nm. In a refinement, the characterizing step includes identifying viral particles such as AAV in at least one of the fractions when the at least one of the fractions exhibits a first wavelength to the second wavelength (first wavelength: second wavelength) absorbance ratio of 1.13 to less than 1.75. In various embodiments, the methods, processes, and systems further include the step of, prior to SEC-MALS analysis, analyzing the plurality of samples by analytical ultracentrifugation to identify the viral particles and/or capsids without encapsidated vector genomes.

Under another aspect, the methods, processes, and systems of various embodiments include the steps of analyzing by SEC and SEC-MALS a plurality of samples from a preparation of viral particles having vector genomes encapsulated within capsids and from the SEC and SEC-MALS analysis, monitoring the structural integrity of the capsids in each of the samples. Each of the samples is modified to have a property that is different from the others. For example, the property is storage of the samples of lengths of time at 25 degrees Celsius (° C.). Also, the changes in protein and nucleic acid concentrations between the samples correlate to changes in the structural integrity of the capsids. The SEC analysis includes the steps of fractioning the sample by size exclusion chromatography, measuring properties of the viral particles, and characterizing the viral particles. The measurements include measuring light absorption by the fractions at wavelengths of about 260 nm and about 280 nm. In a refinement, the characterizing step includes identifying viral particles such as AAV in at least one of the fractions when the at least one of the fractions exhibits a first wavelength to the second wavelength (first wavelength: second wavelength) absorbance ratio of 1.13 to less than 1.75. The SEC-MALS analysis includes the steps of fractioning the sample by size exclusion chromatography, measuring properties of the viral particles, and characterizing the viral particles. The measurements including the refractive indexes of the fractions, the light absorption by the fractions at a wavelength within the ultraviolet spectrum, and the intensity of light scatter by the fractions using MALS. In various embodiments, the methods, processes, and systems further include the step of, prior to SEC-MALS analysis, analyzing the plurality of samples by analytical ultracentrifugation to identify the viral particles and/or capsids without encapsidated vector genomes.

### DESCRIPTION OF DRAWINGS

FIG. 1 is a graph showing light, intermediate, and heavy capsid species in AAV samples. The graph depicts sedimentation of 0% and 100% light capsid material from analytical ultracentrifugation. The sedimentation of light capsids is depicted by the ˜50-60 S peak, while heavy capsids sediment ˜80-100 S. Intermediate capsids are depicted by the shoulder of the heavy capsid peak ˜70-80 S.

FIGS. 2A, 2B, 2C, 2D, 2E, and 2F are graphs of titer calculations from size-exclusion chromatography UV absorbance values. FIG. 2A shows 280 nm and 260 nm absorbance profiles of a heavy AAV sample. FIG. 2B shows 280 nm and 260 nm absorbance profiles of a light AAV sample. FIG. 2C shows a linear regression model fitted to capsid titer. FIG. 2D shows Vg titer as a function of light capsid content (n=3). FIG. 2E shows an underestimation in capsid titer and overestimation in Vg titer calculated from UV absorbance values as a function of light capsid content, fit to exponential and linear regression models, respectively (n=3). FIG. 2F shows a polynomial regression model fit to 260 nm and 280 nm absorbance ratio as a function of light capsids (n=3).

FIGS. 3A and 3B are graphs of capsid and vector genome standard curves for titer calculation by size-exclusion chromatography. FIG. 3A shows a plot of the 280 nm absorbance versus capsid loading (n=3). FIG. 3B shows a plot of the 260 nm absorbance versus vector genome loading (n=3).

FIGS. 4A, 4B, 4C, and 4D are graphs highlighting that multi-angle light scattering measures mass and molar mass of capsid and encapsulated DNA and enables calculation of titers. FIG. 4A is a light scattering chromatogram of a heavy AAV sample with the molar mass of the capsid protein and encapsulated DNA. FIG. 4B is a light scattering chromatogram of a light AAV sample with the molar mass of the capsid protein and encapsulated DNA. FIG. 4C shows a linear regression model fit to mass of capsid and encapsulated DNA, respectively as a function of light capsid content (n=3). FIG. 4D shows a linear regression model fit to molar mass of capsid and encapsulated DNA, respectively as a function of light capsid content (n=3).

FIGS. 5A, 5B, 5C, and 5D are graphs showing an accounting for light and intermediate capsids in multi-angle light scattering titer calculations. FIG. 5A shows a trend in protein fraction from multi-angle light scattering with light-capsid content (n=3). FIG. 5B shows a plot of heavy-capsid percentage calculated from light scattering masses vs expected light capsid percentage fit to a linear regression model (n=3). FIG. 5C shows a linear regression model fit to vector capsid and vector genome titer as a function of light capsids (n=3). FIG. 5D shows the capsid/vector genome ratio calculated from multi-angle light scattering vs expected values of samples containing 0-100% light capsids fit to linear regression models (n=3).

FIGS. 6A and 6B are graphs showing the difference in MALS-derived capsid and vector genomes from theoretical values. The percent difference in capsid and vector genomes from theoretical values was calculated for AAV samples containing 0-100% light capsids (n=3). The shaded region highlights the ±5% difference from the theoretical value.

FIGS. 7A, 7B, 7C, 7D, 7E, and 7F are graphs showing an SEC-MALS analysis of heavy and light capsid thermal stability. FIG. 7A shows 280 nm absorbance profiles of heavy capsid samples incubated at 10° intervals from 25° C. to 95° C. FIG. 7B shows 280 nm absorbance profiles of light capsid samples incubated at 10° intervals from 25° C. to 95° C. FIG. 7C shows a Boltzmann sigmoidal regression model fit to the monomer 280 nm peak absorbance percentage of heavy and light capsids as a function of temperature (n=3). The V50 value (50.03° C.) of the heavy capsid model is indicated by the vertical dotted line. FIG. 7D shows a Monomer peak A60/A280 for heavy and light capsids as a function of temperature (n=3). The V50 value (61.22° C.) of the Boltzmann sigmoidal regression model fit to the heavy capsid data is indicated by the vertical dotted line. FIG. 7E shows the hydrodynamic radius (Rh) and radius of gyration (Rg) of monomeric heavy and light capsid species as a function of temperature (n=3). FIG. 7F shows the Molar mass of heavy and light capsid protein and encapsidated DNA as a function of temperature (n=3). The V50 value (57.89° C.) of the Boltzmann sigmoidal regression model fit to the heavy capsid DNA data is indicated by the vertical dotted line.

FIG. 8A shows a representative SEC and high-performance liquid chromatography (HPLC) profile. The inset figure shows a 100× magnification of the labelled peaks at 260 nm and inset table identifies each peak present in the profile. Each peak was also identified using PCR based methods.

FIG. 8B shows a representative elution profile of AAV capsid particles by SEC.

FIGS. 9A, 9B, and 9C show analysis of capsid stability by monitoring the change in the SEC peak area. Adeno-associated virus (AAV) samples were stored at 25° C. for 0, 1, 3, 5, 7, 10, 14, 21, and 28 days. The % peak area of the extraneous deoxyribonucleic acid (DNA) peak increased linearly by about 2-fold, which suggests a change in the stability of the capsids.

FIGS. 10A, 10B, and 10C show multiangle light scattering (MALS) analysis of SEC eluted capsids and encapsidated vector genomes.

FIGS. 11 and 12 show examples of data acquired from SEC-MALS analysis of the AAV samples.

FIGS. 13A, 13B, and 13C are graphs highlighting the DNA mass of AAV particles decreasing linearly with increasing concentrations of light capsids, while protein mass remains constant as the total concentration of capsid (full or empty) does not change.

FIG. 14A is a graph highlighting the total molecular weight (MW) of the AAV particles (capsid and DNA) as measured by MALS, show a decrease in MW with increasing concentrations of light capsids.

FIGS. 14B and 14C are graphical representation showing that the decrease in the total MW of the AAV particles as shown in FIG. 14A is due to decreasing MW of the DNA portion. FIGS. 14B and 14C also show that the molecular weight of the DNA portion decreases linearly with increasing concentrations of light capsids while the molecular weight of the capsid remains constant regardless of the portion of light capsids.

FIG. 15 is a fluorescent image of an ethidium bromide stained agarose gel of encapsidated DNA from AAV samples.

FIG. 16 is a graph showing the sedimentation of light, intermediate, and heavy capsids.

FIGS. 17A, 17B, and 17C are graphs showing the percentage of full capsid concentration, capsid concentration, and vector concentration relative to the percentage of light capsids.

FIGS. 18A and 18B are graphs showing comparisons of MW and size distributions among different AAV samples as calculated by MALS.

FIG. 19 is a graph showing an estimation of the percentage of empty capsid particles using SEC-MALS data.

FIGS. 20A, 20B, 20C and 20D are graphs showing estimations of capsid and vector genome titers of AAV particles using SEC-MALS.

FIG. 21 provides SEC elution profiles of LV samples. Fractions 1-12 were collected after injection of purified LV sample and circled fractions were selected for p24 and droplet digital PCR (ddPCR) analysis. These analyses confirmed presence of LV particles eluting in the void volume of the column, represented by the peak around the 19 minute mark. Remaining peaks between 25 and 45 minutes represent protein or nucleic acid sample impurities.

FIG. 22 provides the effect of buffer salt concentration on LV SEC profile. SEC elution profiles, detected by UV absorbance at 280 nm, following 50 μL injections of purified LV sample. 20 mM Tris at pH 7.40 with either 300 mM NaCl (bold line) or 150 mM NaCl (dashed line) was used as an elution buffer and a flow rate of 0.300 mL/min was applied.

FIGS. 23A and 23B provide SEC-MALS elution profiles of LV sample. FIG. 23A provides the mean SEC elution profile, detected by UV absorbance at 280 nm, obtained after triplicate 40 μL injections of LV sample. FIG. 23B provides the corresponding mean MALS profile obtained from the same sample injections. The prominent light scattering peak around the 17 minute mark further supports p24 and ddPCR data indicating LV particle elution in the void volume of the column.

FIGS. 24A and 24B provide a linearity model of MALS number density analysis of LV samples. FIG. 24A provides mean MALS peak profiles obtained after triplicate 10 μL, 20 μL, 40 μL, and 80 μL injections of LV sample. While the average MW of particles eluting at 17 minutes was consistent (˜1.25×108 Da) regardless of injection volume, the intensity of the MALS signals was proportional to the volume of sample injected. FIG. 24B provides the calibration curve of the SEC-MALS method, correlating number density measured by SEC-MALS to sample injection volume. Validity of the linear model was confirmed by F-test statistics at the 95% confidence interval with an average correlation coefficient of 0.9947.

FIGS. 25A and 25B provide SEC-MALS pH stability analysis of LV particles. FIG. 25A provides the mean SEC elution profile, detected by UV absorbance at 280 nm, obtained after triplicate 25 μL injections of LV particles dialyzed to pH 4.00, pH 7.40, and pH 10.00. Elution of capsids is represented by the peak observed ˜17 minutes after injection. Compared to the SEC profile of pH 7.00 LV particles, the pH 4.00 LV peak is almost negligible while the pH 10.00 peak is enlarged. FIG. 25B provides mean MALS peak profiles displaying the same trend seen in the SEC profiles described above.

FIG. 26 provides a circular dichroism (CD) secondary structure analysis of LV particle proteins as a function of pH. Overlain CD curves of LV particles at pH 4.00, 7.40, and 10.00. Curve characteristics, including double minima at 210 nm and 220 nm, suggest a predominantly alpha-helical conformation in LV particles at pH 7.40 and 10.00. This conformation is entirely lost at pH 4.00.

FIGS. 27A and 27B provide dynamic light scattering (DLS) melting curves of LV particles as a function of pH. FIG. 27A provides a comparison of thermal curves of LV particles at pH 6.00, pH 7.40, and pH 10.00, derived by monitoring the hydrodynamic diameter of LV particles assessed by DLS as a function of temperature. The melting temperature of each pH sample is depicted by the vertical dotted lines. FIG. 27B provides a bar graph depicting the statistical significance of differences in LV melting temperature as a function of pH.

FIGS. 28A and 28B provide SEC-MALS salt stability analysis of LV particles. FIG. 28A provides a mean SEC elution profile, detected by UV absorbance at 280 nm, obtained after triplicate 25 uL injections of LV particles dialyzed to 0 mM, 300 mM, and 1 mM NaCl. Elution of capsids is represented by the peak observed ˜17 minutes after injection. FIG. 28B provide the mean MALS peak profiles corresponding to the SEC profiles described above in FIG. 28A.

FIG. 29 provides CD curves of LV salt samples.

FIGS. 30A and 30B provide the DLS thermal ramp of LV particles as a function of salt. FIG. 30A provides a comparison of the thermal curves of LV particles at 0 mM, 300 mM, and 1 M NaCl, derived by monitoring the hydrodynamic diameter of LV particles assessed by DLS as a function of temperature. The melting temperature of each salt sample is depicted by the vertical dotted lines. FIG. 30B provides a bar graph depicting the statistical significance of differences in LV melting temperature as a function of salt.

FIGS. 31A, 31B, 31C, and 31D provide an initial thermal stability analysis of empty and full rAAV5 capsids using intrinsic fluorescence and circular dichroism. FIG. 31A shows 1st derivatives of representative intrinsic fluorescent curves of empty and full capsids showing a melting temperature of proteins from both capsids around 90° C., consistent with previously reported AAV5 melting temperatures determined by DSC. FIG. 31B provides representative Far-UV CD spectra of empty and full capsids, showing differences in absorbance at 210 nm and 270 nm. Prominent minimum around 210 nm observed in empty-capsid spectrum indicates more of an alpha-helical conformation of empty-capsid proteins than full-capsid proteins. DNA encapsulated by full capsids showed absorption at 270 nm, not seen in empty capsids which are not encapsulating DNA. FIG. 31C provides a comparison of the melting curve of empty and full capsids, derived by monitoring the CD ellipticity at 220 nm as a function of temperature. The melting temperature of both capsid types around 90° C. is depicted by the vertical dotted line, while the start of the biphasic event observed only in full capsids is indicated by the arrow. FIG. 31D provides a comparison of the melting curve of empty and full capsids, derived by monitoring the CD ellipticity at 270 nm as a function of temperature. Again, the melting temperature of both capsid types, indicated by the vertical dotted line, is 90° C., while the arrow indicates a biphasic event observed before the melting temperature exclusively in full capsids.

FIGS. 32A, 32B, 32C, and 32D provides SEC-MALS thermal stability analysis of empty and full rAAV5 capsids. FIG. 32A provides the mean SEC elution profile, detected by UV absorbance at 280 nm, obtained after triplicate injections of empty capsids incubated at 10° C. intervals from 25° C. to 95° C. Elution of capsids is represented by the prominent peak observed ˜11 minutes after injection. SEC profile of empty capsids remains relatively constant, with limited reduction in main peak size up to 75° C., followed by a sharp decline due to the melting of protein capsids between 85° C. and 95° C. FIG. 32B provides the mean SEC UV 280 nm elution profile obtained after triplicate injections of full capsids incubated at respective temperatures. Changes in SEC profile of full capsids begin at 45° C., with a drop in prominent peak size observed between 45° C. and 65° C. FIG. 32C provides the percentage of main UV peak for both empty and full capsids was measured and plotted to show decline in capsid integrity as a function of temperature. FIG. 32D provides the percentage of main MALS peak for both empty and full capsids, mirroring the same trend as that seen in UV peak percentage.

FIGS. 33A and 33B provide MALS analysis of capsid protein and encapsulated DNA molecular weight as a function of temperature. FIG. 33A shows the molecular weight of the protein capsid as a function of temperature for both empty and full capsids as a function of temperature. FIG. 33B shows the molecular weight of the encapsulated DNA as a function of temperature for both empty and full capsids.

FIGS. 34A, 34B, and 34C provide extrinsic fluorescent analysis of empty and full rAAV5 capsids using SYBR gold dye. FIG. 34A provides an extrinsic fluorescence assay scheme. FIG. 34B shows the fluorescence spectrum of SYBR gold dye mixed with full capsids as a function of temperature. FIG. 34C shows the total area under the fluorescent curves was measured and plotted as a function of temperature for both full and empty capsids. A biphasic fit was obtained for full capsids, while empty capsids did not show any trend on this plot. The vertical dotted lines represent half maximum of the two transition temperatures as observed for full capsids.

FIGS. 35A and 35B provide agarose gel images of in-house and ViGene AAV5 samples. FIG. 35A shows an agarose gel image of AAV5 capsids coating a range of single-stranded genome sizes. FIG. 35B shows an agarose gel image of AAV5 capsids coating a 3.5 kb or 4.5 kb genome, obtained from ViGene Biosciences.

FIGS. 36A, 36B, 36C, and 36D provide extrinsic fluorescent analysis to determine the effect of DNA load on rAAV5 capsid integrity. FIGS. 36A and 36C provide fluorescence spectra of SYBR gold dye mixed with capsids having variable size of encapsulated DNA was obtained and the area under the curve was calculated and plotted as a function of temperature for capsids from Source A and Source B. FIGS. 36B and 36D provide bar graphs depicting the statistically significant differences in rAAV5 capsid transition temperature as a function of encapsulated genome size for capsids from Source A and Source B. As the size of the encapsulated genome increases, the transition temperature, indicative of capsid breakdown, decreases.

FIGS. 37A, 37B, 37C, and 37D provide SEC analysis to establish the effect of increasing DNA load on rAAV5 capsid integrity. SEC profile of sample 2 capsids (FIG. 37A), sample 4 capsids (FIG. 37B), and sample 6 capsids (FIG. 37C) were subjected to 25° C., 55° C., and 75° C. for 30 minutes. Samples were selected based on increasing DNA load of the capsids as shown by the alkaline gel picture in FIG. 22 herein. While all samples displayed similar profiles at 25° C. (uniform rAAV5 capsid peak) and 75° C. (<10% of the original peak), the profiles of samples obtained at 55° C. varied based on the different transition temperatures for each of capsids in each of the three samples based on the size of their encapsulated genome.

### DETAILED DESCRIPTION

Herein, the biophysical characterization of viral particles such as AAV or LV was achieved through the use of SEC or SEC-MALS. In particular, the methods were used to quantify aggregation of the viral particles in a preparation, quantify a concentration of nucleic acid and/or protein impurities in the preparation, determining weight averaged molecular weights of the capsids and vector genomes, quantify concentrations of viral particles and capsids devoid of encapsulated vector genomes in the preparation, determine a size distribution of the viral particles and/or the capsids devoid of encapsulated vector genomes in the preparation, and monitor the structural integrity of the capsids.

Also, the biophysical characterization of AAV (non-enveloped) or LV (enveloped) particles was achieved through the use of SEC-MALS, CD, DLS, and fluorescence to assess capsid structure. In particular, these methods were used to determine viral particle size and viral capsid destabilization. There is a positive correlation between capsid integrity and pH. Additionally, high ionic conditions serve to stabilize LV capsids. Lastly, a role of the encapsulated genome size in regulating AAV capsid thermal stability was identified. The combined use of these biophysical tools (SEC and MALS) serves as an effective means for comprehensive characterization and structure-function elucidation of viral vectors used for gene therapy.

### General Techniques

The practice of the present methods will employ, unless otherwise indicated, conventional techniques of cell biology, molecular biology, cell culture, virology, and the like which are in the skill of one in the art. These techniques are fully disclosed in current literature and reference is made specifically to Sambrook, Fritsch and Maniatis eds., “Molecular Cloning, A Laboratory Manual”, 2nd Ed., Cold Spring Harbor Laboratory Press (1989); Celis J. E. “Cell Biology, A Laboratory Handbook” Academic Press, Inc. (1994) and Bahnson et al., J. of Virol. Methods, 54:131-143 (1995). Furthermore, all publications and patent applications cited in this specification are indicative of the level of skill of those skilled in the art to which these methods pertains and are hereby incorporated by reference in their entirety.

### Definitions

Throughout the present disclosure, several terms are employed that are defined in the following paragraphs.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.”

As used herein, the term “about” when applied to values indicates that the calculation or the measurement allows some slight imprecision in the value (with some approach to exactness in the value; approximately or reasonably close to the value; nearly). If, for some reason, the imprecision provided by “about” is not otherwise understood in the art with this ordinary meaning, then “about” as used herein indicates at least variations that may arise from ordinary methods of measuring or using such parameters.

As used herein, the term “and/or” includes any and all combinations of one or more of the associated listed items.

The term “viral particle” or “virion” refers to an RNA or DNA core with a protein coat and depending on the virus the core and protein may comprise an external envelope. Exemplary viral particles are AAV particles, LV particles, adenovirus particles, alphavirus particles, herpesevirus particles, retrovirus particles, and vaccinia virus particles.

The term “capsid particle” refers to protein coat that may or may not contain an RNA or DNA core. For example, a capsid particle that does not contain an RNA or DNA core can also be described as an empty or light capsid particle or an empty or light capsid. A capsid particle that does contain an RNA or DNA core can also be described as a full capsid particle or viral particle or virion depending on the type of virus (e.g. viruses without external envelopes).

As used herein, an “AAV vector” refers to nucleic acids, either single-stranded or double-stranded, having an AAV 5′ inverted terminal repeat (ITR) sequence and an AAV 3′ ITR flanking a protein-coding sequence (preferably a functional therapeutic protein-encoding sequence; e.g., FVIII, FIX, and PAH) operably linked to transcription regulatory elements that are heterologous to the AAV viral genome, i.e., one or more promoters and/or enhancers and, optionally, a polyadenylation sequence and/or one or more introns inserted between exons of the protein-coding sequence. A single-stranded AAV vector refers to nucleic acids that are present in the genome of an AAV virus particle, and can be either the sense strand or the anti-sense strand of the nucleic acid sequences disclosed herein. The size of such single-stranded nucleic acids is provided in bases. A double-stranded AAV vector refers to nucleic acids that are present in the DNA of plasmids, e.g., pUC19, or genome of a double-stranded virus, e.g., baculovirus, used to express or transfer the AAV vector nucleic acids. The size of such double-stranded nucleic acids in provided in base pairs (bp).

An “AAV virion” or “AAV viral particle” or “AAV vector particle” or “AAV virus” refers to a viral particle composed of at least one AAV capsid protein and an encapsidated polynucleotide AAV vector as described herein. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector”. Thus, production of AAV vector particles necessarily includes production of AAV vector, as such a vector is contained within an AAV vector particle.

The term “lentivirus” refers to a group of complex retroviruses, while the term “recombinant lentivirus” refers to a recombinant virus derived from lentivirus genome (such as an HIV-1 genome) engineered such that it cannot replicate but can be produced in cultured cells (e.g., 293T cells) and can deliver genes to cells of interest.

The term “vesiculovirus” refers to a genus of negative-sense single stranded retrovirus in the family of Rhabdoviridae.

The term “envelope protein” refers to a transmembrane protein on the surface of a virus that determines what species and cell types the virus can transduce.

The term “pseudotyping” refers to the replacement of any component of a virus with that from a heterologous virus. In particular, “pseudotyping” denotes a recombinant virus comprising an envelope different from the wild-type envelope, and thus possessing a modified tropism. In the case of the pseudotyped lentiviruses, they are lentiviruses which have a heterologous envelope of non-lentiviral origin or a different species or subspecies of lentivirus, for example originating from another virus, or of cellular origin, or the envelope is replaced with another cellular membrane protein originating from another virus or cellular origin

The terms “VSV envelope” refers to an envelope protein from a rhabdovirus called vesicular stomatitis virus (VSV). Often this protein is also referred to as the VSV-G protein where “G” means glycoprotein. The envelope protein of rhabdoviruses is the only rhabdovirus protein that is glycosylated.

“Size exclusion chromatography” (SEC), also known as “gel filtration chromatography” refers to a chromatography method which separates molecules based on their size by filtration through a gel.

The term “fractionation” or “fractioning” refers to separation of molecules of varying molecular weights within the SEC gel matrix. With this separation method, the molecules of interest should fall within the fractionation range of the gel. The term “fraction” refers to a peak of molecules eluted off the SEC gel matrix.

The term “flow rate” refers to the volume of fluid that is passing through a given cross sectional area of the SEC column per unit time. In general, moderate flowrates offer the highest resolution. Flowrates are specific to the type of media being used. Moderate flow rates allow time for the molecules to fully access the surface area of the stationary phase permitting the smaller MW species the time to enter the pores, resulting in improved partitioning of the different MW species. Flowrates that are too slow will reduce resolution since the peaks or bands will diffuse too much as they travel through the column.

“Multiangle light scattering” (MALS) refers to a technique for measuring the light scattered by a sample into a plurality of angles. This technique is useful for determining both the absolute molar mass and the average size of molecules in solution, by detecting how they scatter light. The “multi-angle” term refers to the detection of scattered light at different discrete angles as measured, for example, by a single detector moved over a range that includes the particular angles selected or an array of detectors fixed at specific angular locations. MALS measurements are generally expressed as scattered intensities or scattered irradiance.

The “refractive index increment” or “dn/dc” is a constant indicating the variation of the refractive index with the viral particle, capsid, or vector genome. The refractive index increment is used in Snell's law to measure concentrations from the refractive index (RI). For example, the dn/dc for protein is 0.185 and the dn/dc for DNA is 0.170. The refractive index increment can be determined by AUC.

The “extinction coefficient”, “molar extinction coefficient”, or “ε” is a measure of how strongly a chemical species or substance absorbs light at a particular wavelength. The extinction coefficient is used in Beer-Lambert law to measure concentrations from light absorbance at a wavelength (e.g. 280 nm). For example, the extinction coefficient for the AAVS capsid is 1.79 and the extinction coefficient for an exemplary vector genome is 17.0.

Empty viral particles, also referred to as “empty capsids” or “light capsids” refer to viral particles that contain low amounts or no viral genome DNA. Empty capsids that are typically formed during AAV or LV vector production. These empty viral particles may copurify with genome-containing vector particles during chromatographic purification, and the excess of empty capsids confounds simple determinations of vector genome concentration by absorbance.

Capsid particle (Cp) titer refers to the number of viral particles per milliliter.

Vector genome (Vg) titer refers to the number of viral genomes per milliliter. This titer may be determined by the ratio of viral particle absorbance at UV 260/UV 280. The absorbance (A260) of a highly purified AAV preparation depends on the MW of the vector DNA and the amount of capsid protein.

The “radius of gyration (Rg)” also known as the “root mean square radius” refers to the measurement of absolute molar mass of the viral particles in solution as measured by MALS. This measurement is determined by the mass weighted average distance from the core of a molecule to each mass element in the viral particle. The Rg can be determined by dynamic light scattering analysis.

The “hydrodynamic radius (Rh)” is the radius of an equivalent hard sphere diffusing at the same rate as the viral particle under observation. The Rh is measured by dynamic light scattering in the MALS detector. As the solutions of viral particles do not exist as “hard spheres” so, the determined Rh reflects the apparent size adopted by the viral particle in solution. The Rh can be determined by dynamic light scattering analysis.

### Methods of Characterizing Viral Particles

The present disclosure provides for combined method utilizing SEC and SEC-MALS techniques. These methods provide a robust and direct approach for quantification of multiple attributes of AAV and LV particles. These methods exploit the absorbance and light scattering properties of capsid and encapsidated DNA to deduce the total capsid particle (cp) and encapsidated vector genome (vg) content in solution. Furthermore, these methods determine the average molecular mass of the viral particles and the encapsidated vector genome, the size distribution and aggregation profile of the viral particles, the amount of extrinsic DNA, the capsid integrity as well as the ratio of empty to full viral particles in purified AAV or LV preparations. Currently multiple techniques are being used to generate the same amount of information with varying accuracy and precision. The disclosed methods utilize intrinsic properties of the capsid particle and encapsidated vector genome that provides critical information and quantifies numerous physical characteristics of an AAV or LV solution in one run with high precision and minimal manipulation of the sample. Data generated through these methods were compared with orthogonal techniques and results demonstrate that the SEC-MALS assay is able to determine various quality attributes of AAV or LV rapidly and with high precision. This well-established method with novel applications is a powerful tool for product development and process analytics in the field of AAV gene therapy.

Currently multiple techniques are being used to generate the same amount of information with varying accuracy and precision. The disclosed methods utilize the intrinsic properties of the viral particle and encapsidated vector genome that provides critical information and quantifies numerous physical characteristics of an AAV or LV solution in one run with high precision and minimal manipulation of the sample.

Comprehensive biophysical characterization of both intermediate and final viral vector products is crucial in their rational design and development for biomedical applications. Further, biophysical tools can be used to elucidate viral vector structure-function relationships. Consequently, initial work with LV samples in the present study focused on the development of an LV characterization method, with method-design rationale centering on the use of biophysical tools to qualitatively and quantitatively measure parameters, including heterogeneity, size, and titer of LV particles in real time.

Currently, SEC, also known as gel-filtration chromatography, is widely-used as an industry workhorse to quickly and reproducibly evaluate biotherapeutics with minimal cost and effort. SEC is a powerful technique which provides information about the heterogeneity, distribution, and aggregation of biomolecules in a sample. SEC separates molecules in solution by their size or weight in solution, with larger species eluting from the column faster than smaller ones.

In general, the SEC gel consists of spherical beads containing pores of a specific size distribution. Separation occurs when molecules of different sizes are included or excluded from the pores within the matrix. Small molecules diffuse into the pores and their flow through the column is retarded according to their size, while large molecules do not enter the pores and are eluted in the column's void volume. Consequently, molecules separate based on their size as they pass through the column and are eluted in order of decreasing molecular weight (MW).

Operating conditions and gel selection depend on the application and the desired resolution. Two common types of separations performed by SEC are fractionation and desalting (or buffer exchange.) The resolution of separation depends on particle size, pore size, flow rate, column length and diameter, and sample volume. In general, the smaller the particle size, the higher the resolution. Pore size controls the exclusion limit and the fractionation range of the media. Resolution increases with the column length, and as the column diameter increases, the capacity of the column increases due to the larger column or bed volume. Column eluates are then detected by an UV detector, in this case at 280 nm, the wavelength of light that proteins in solution absorb. Though this tool offers many advantages in evaluating viral samples, an important handicap is the qualitative vs quantitative nature of the data it provides. However, this can be augmented by combining SEC with a quantitative biophysical tool.

Column packing is critical to resolution; an overpacked column can collapse the pores in the beads resulting in diminished resolution. An underpacked column increases the mixing volume outside of the pores, resulting in broader, less resolved, peaks. Dead volume at the top of the column can significantly reduce resolution as the sample is allowed to diffuse prior to entering the column bed, resulting in “band broadening” or wider peaks. Dead volume at the top of the column is possibly the most critical consideration because the loss in resolution is then multiplied as the molecules travel through the column.

Exemplary columns that can be used in the disclosed methods include TSKgel G5000PWXL (TOSOH Bioscience), Superdex 200 10/300 GL, Sepax SEC 1000 column (Sepax technologies), Superdex 200 (GE Lifescience), or Superdex XK26/60 (GE Healthscience) qEV size exclusion columns (Izon Scientific). The columns selected for separation of AAV or LTs is able to separate proteins in the 100 kilodaltons (kDa) to 10 megadaltons (MDa) range or with hydrodynamic sizes between 10 and 40 nm.

Analysis of an elution profile of an AAV or LT particle on SEC indicates that the first and second peaks at about UV260:UV280 are extrinsic DNA, the third peak represents dimers of the viral particles, fourth peak represents the monomer viral particles, the fifth peak represents small nucleotides and buffer components. Though SEC allows for qualitative analysis of AAV and LV vector samples, inability to quantitatively assess the gene therapy vectors is a substantial drawback of this method. These quantitative analytical measurements, including total virus particle count and size, are key to effectively gain understanding of the biophysical parameters of viral vectors. Therefore, in developing an AAV or LV biophysical characterization method, the coupling of SEC to MALS was evaluated as a means of broadening the scope of accessible data.

In the present study, the coupling of SEC to MALS for the directly quantification of biomolecules eluting from the SEC column. MALS, which measures light scattered by molecules in solution at multiple angles, uses the intensity of the scattered light to extricate the molecular weight, size, and number of the light-scattering molecules. The light scattering principle delineates that the intensity of the MALS peak is equivalent to the light-scattering molecule's weight squared.

Size exclusion chromatography with multi-angle static light scattering is referred to herein as “SEC-MALS”. SEC separates molecules based on hydrodynamic volume, but is dependent on similarity to a set of reference standards for accurate mass determination. MALS uses the intensity and the angular dependence of the scattered light to measure absolute molar mass and size of the molecules (root mean square radius, rg) in solution. The number of angles in a MALS system can vary between 2 up to 20 angles, where the scattering is detected simultaneously at each angle. While any light scattering detector (single or multi angle) can measure molecular weight, the main benefit of obtaining light scattering data as a function of scattering angle is that the Rg or root mean squared (RMS) radius can be calculated to give the size of molecules. Combining SEC and MALS in an SEC-MALS experiment allows for more accurate mass measurements that either method alone. An inline Quasi-Elastic Light Scattering (QELS), also called a Dynamic Light Scattering (DLS) detector, enables measurement of a hydrodynamic radius.

The large size of LV and AAV particles, theoretically enabling them to scatter plenty of light, makes MALS an attractive tool in LV and AAV particle analysis. Another advantage of MALS is that, as an absolute method, it does not require preparation of a calibration curve. Using a method previously outlined by Steppert et al., (J Chromatogr A. 2017; 1487:89-99) for the characterization of virus-like particles as a starting point, a SEC-MALS method to biophysically analyze LV particles was developed and is disclosed herein. Notably, the disclosed method not only allowed for qualitative and quantitative evaluation of AAV or LV particles, but offered a new, reproducible way to rapidly estimate LV particle titer.

In any of the disclosed methods, SEC-MALS is carried out using at with least 2 angles, at least 3 angles, at least 4 angles, at least 5 angles, at least 6 angles, at least 7 angles, at least 8 angles, at least 9 angles, at least 10 angles, at least 11 angles, at least 12 angles, at least 13 angles, at least 14 angles, at least 15 angles, at least 16 angles, at least 17 angles, at least 18 angles, at least 19 angles, at least 20 angles to determine the molecular weight of the AAV or LV.

In any of the methods described herein, SEC-MALS is carried out with a flow rate ranging from about 0.1 ml/ml to 1.5 ml/min, or a flow rate ranging from about 0.3 ml/min to about 1.0 ml/min, or a flow rate ranging from 0.3 ml/min to about 0.5 ml/min, or a flow rate ranging from 0.5 ml/min to about 1.0 ml/min. For example, the SEC-MALS is carried out with a flow rate of about 0.1 ml/min., about 0.2 ml/min., about 0.3 ml/min, about 0.4 ml/min, about 0.5 ml/min, about 0.6 ml/min, about 0.7 ml/min, about 0.8 ml/min, about 0.9 ml/min, about 1.0 ml/min, about 1.1 ml/min, about 1.2 ml/min, about 1.3 ml/min, about 1.4 ml/min, about 1.5 ml/min, about 1.6 ml/min, about 1.7 ml/min, about 1.8 ml/min, about 1.9 ml/min, about 2.0 ml/min.

### Recombinant AAV Particles

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228; and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, e.g., Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “rAAV viron” or “rAAV viral particle” or “rAAV vector particle” or “AAV virus” refers to a viral particle composed of at least one capsid or Cap protein and an encapsidated rAAV vector genome as described herein. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV vector”. Thus, production of AAV vector particles necessarily includes production of rAAV vector, as such a vector is contained within an rAAV vector particle.

A therapeutically effective AAV particle or therapeutic AAV virus is capable of infecting cells such that the infected cells express (e.g. by transcription and/or by translation) an element (e.g. nucleotide sequence, protein, etc.) of interest. To this extent, the therapeutically effective AAV particles can include AAV particles having capsids or vector genomes (vgs) with different properties. For example, the therapeutically effective AAV particles can have capsids with different post translation modifications. In other examples, the therapeutically effective AAV particles can vgs with differing sizes/lengths, plus or minus strand sequences, different flip(5′ ITR)/flop(3′ ITR) ITR configurations (e.g. 5′ ITR/3′ ITR, 3′ ITR/5′ ITR, 3′ ITR/3′ ITR, 5′ ITR/5′ ITR, etc.), different number of ITRs (1, 2, 3, etc.), or truncations. For example, overlapping homologous recombination occurs in AAV infected cells between nucleic acids having 5′ end truncations and 3′ end truncations so that a “complete” nucleic acid encoding the large protein is generated, thereby reconstructing a functional, full-length gene. Therapeutically effective AAV particles are also referred to as “heavy” or “full” capsids.

As an example, a “therapeutic AAV virus”, which refers to an AAV virion, AAV viral particle, AAV vector particle, or AAV virus that comprises a heterologous polynucleotide that encodes a therapeutic protein, can be used to replace or supplement the protein in vivo. The “therapeutic protein” is a polypeptide that has a biological activity that replaces or compensates for the loss or reduction of activity of a corresponding endogenous protein. For example, a functional phenylalanine hydroxylase (PAH) is a therapeutic protein for phenylketonuria (PKU). Thus, for example recombinant AAV PAH virus can be used for a medicament for the treatment of a subject suffering from PKU. The medicament may be administered by intravenous (IV) administration and the administration of the medicament results in expression of PAH protein in the bloodstream of the subject sufficient to alter the neurotransmitter metabolite or neurotransmitter levels in the subject. Optionally, the medicament may also comprise a prophylactic and/or therapeutic corticosteroid for the prevention and/or treatment of any hepatotoxicity associated with administration of the AAV PAH virus. The medicament comprising a prophylactic or therapeutic corticosteroid treatment may comprise at least 5, 10, 15, 20, 25, 30, 35, 40, 45, 50, 55, 60, or more mg/day of the corticosteroid. The medicament comprising a prophylactic or therapeutic corticosteroid may be administered over a continuous period of at least about 3, 4, 5, 6, 7, 8, 9, 10 weeks, or more. The PKU therapy may optionally also include tyrosine supplements.

Therapeutically ineffective AAV particles are incapable of infecting cells or a cell infected with therapeutically ineffective AAV particles are unable to express (e.g. by transcription and/or by translation) an element (e.g. nucleotide sequence, protein, etc.) of interest. Therapeutically ineffective AAV particles can contribute to decreased effectiveness per unit dose of capsid and can increase the risk of an immune response due to a needed increased amount of foreign proteins being introduced into the patient for an effective amount of heavy/full capsid. Therapeutically ineffective AAV particles can include AAV particles having capsids or vgs with different properties and are referred to as “partially full” capsids and empty capsids or “light” capsids that include both partially full and empty capsids. For example, empty capsids do not have a vg or have an unquantifiable vg concentration. Empty capsids can also have different capsid properties. While not being bound to by any particular theory, the heavy/full capsids differ from partially full or empty capsids in their charge and/or density.

AAV “rep” and “cap” genes are genes encoding replication and encapsidation proteins, respectively. AAV rep and cap genes have been found in all AAV serotypes examined to date, and are described herein and in the references cited. In wild-type AAV, the rep and cap genes are generally found adjacent to each other in the viral genome (i.e., they are “coupled” together as adjoining or overlapping transcriptional units), and they are generally conserved among AAV serotypes. AAV rep and cap genes are also individually and collectively referred to as “AAV packaging genes.” The AAV cap genes encode Cap proteins which are capable of packaging AAV vectors in the presence of rep and adeno helper function and are capable of binding target cellular receptors. In some embodiments, the AAV cap gene encodes a capsid protein having an amino acid sequence derived from a particular AAV serotype.

The AAV sequences employed for the production of AAV can be derived from the genome of any AAV serotype. Generally, the AAV serotypes have genomic sequences of significant homology at the amino acid and the nucleic acid levels, provide a similar set of genetic functions, produce virions which are essentially physically and functionally equivalent, and replicate and assemble by practically identical mechanisms. For the genomic sequence of AAV serotypes and a discussion of the genomic similarities. (See, e.g., GenBank Accession number U89790; GenBank Accession number J01901; GenBank Accession number AF043303; GenBank Accession number AF085716; Chiorini et al., J. Vir. (1997) vol. 71, pp. 6823-6833; Srivastava et al., J. Vir. (1983) vol. 45, pp. 555-564; Chiorini et al., J. Vir. (1999) vol. 73, pp. 1309-1319; Rutledge et al., J. Vir. (1998) vol. 72, pp. 309-319; and Wu et al., J. Vir. (2000) vol. 74, pp. 8635-8647).

The genomic organization of all known AAV serotypes is very similar. The genome of AAV is a linear, single-stranded DNA molecule that is less than about 5,000 nucleotides (nt) in length. Inverted terminal repeats (ITRs) flank the unique coding nucleotide sequences for the non-structural Rep proteins and the structural (VP) proteins. The VP proteins form the capsid. The terminal 145 nt are self-complementary and are organized so that an energetically stable intramolecular duplex forming a T-shaped hairpin may be formed. These hairpin structures function as an origin for viral DNA replication, serving as primers for the cellular DNA polymerase complex. The Rep genes encode the Rep proteins, Rep78, Rep68, Rep52, and Rep40. Rep78 and Rep68 are transcribed from the p5 promoter, and Rep 52 and Rep40 are transcribed from the p19 promoter. The cap genes encode the VP proteins, VP1, VP2, and VP3. The cap genes are transcribed from the p40 promoter. The ITRs employed in the disclosed vectors may correspond to the same serotype as the associated cap genes, or may differ. In a particularly preferred embodiment, the ITRs employed in the disclosed vectors correspond to an AAV2 serotype and the cap genes correspond to an AAVS serotype.

In some embodiments, a nucleic acid sequence encoding an AAV capsid protein is operably linked to expression control sequences for expression in a specific cell type, such as Sf9 or HEK cells. Techniques known to one skilled in the art for expressing foreign genes in insect host cells or mammalian host cells can be used herein. Methodology for molecular engineering and expression of polypeptides in insect cells is described, for example, in Summers and Smith (1986) A Manual of Methods for Baculovirus Vectors and Insect Culture Procedures, Texas Agricultural Experimental Station Bull. No. 7555, College Station, Tex.; Luckow (1991) In Prokop et al., Cloning and Expression of Heterologous Genes in Insect Cells with Baculovirus Vectors' Recombinant DNA Technology and Applications, 97-152; King, L. A. and R. D. Possee (1992) The baculovirus expression system, Chapman and Hall, United Kingdom; O'Reilly, D. R., L. K. Miller, V. A. Luckow (1992) Baculovirus Expression Vectors: A Laboratory Manual, New York; W. H. Freeman and Richardson, C. D. (1995) Baculovirus Expression Protocols, Methods in Molecular Biology, volume 39; U.S. Pat. No. 4,745,051; US2003148506; and WO 03/074714, all of which are incorporated by reference in their entireties. A particularly suitable promoter for transcription of a nucleotide sequence encoding an AAV capsid protein is e.g. the polyhedron promoter. However, other promoters that are active in insect cells are known in the art, e.g. the p10, p35 or IE-1 promoters and further promoters described in the above references are also contemplated.

Use of insect cells for expression of heterologous proteins is well documented, as are methods of introducing nucleic acids, such as vectors, e.g., insect-cell compatible vectors, into such cells and methods of maintaining such cells in culture. (See, e.g., METHODS IN MOLECULAR BIOLOGY, ed. Richard, Humana Press, N J (1995); O'Reilly et al., BACULOVIRUS EXPRESSION VECTORS, A LABORATORY MANUAL, Oxford Univ. Press (1994); Samulski et al., J. Vir. (1989) vol. 63, pp. 3822-3828; Kajigaya et al., Proc. Nat'l. Acad. Sci. USA (1991) vol. 88, pp. 4646-4650; Ruffing et al., J. Vir. (1992) vol. 66, pp. 6922-6930; Kirnbauer et al., Vir. (1996) vol. 219, pp. 37-44; Zhao et al., Vir. (2000) vol. 272, pp. 382-393; and U.S. Pat. No. 6,204,059). In some embodiments, the nucleic acid construct encoding AAV in insect cells is an insect cell-compatible vector. An “insect cell-compatible vector” or “vector” as used herein refers to a nucleic acid molecule capable of productive transformation or transfection of an insect or insect cell. Exemplary biological vectors include plasmids, linear nucleic acid molecules, and recombinant viruses. Any vector can be employed as long as it is insect cell-compatible. The vector may integrate into the insect cells genome but the presence of the vector in the insect cell need not be permanent and transient episomal vectors are also included. The vectors can be introduced by any means known, for example by chemical treatment of the cells, electroporation, or infection. In some embodiments, the vector is a baculovirus, a viral vector, or a plasmid. In a more preferred embodiment, the vector is a baculovirus, i.e. the construct is a baculoviral vector. Baculoviral vectors and methods for their use are described in the above cited references on molecular engineering of insect cells.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori (Bm)NPV).

Baculoviruses are commonly used for the infection of insect cells for the expression of recombinant proteins. In particular, expression of heterologous genes in insects can be accomplished as described in for instance U.S. Pat. No. 4,745,051; Friesen et al (1986); EP 127,839; EP 155,476; Vlak et al (1988); Miller et al (1988); Carbonell et al (1988); Maeda et al (1985); Lebacq-Verheyden et al (1988); Smith et al (1985); Miyajima et al (1987); and Martin et al (1988). Numerous baculovirus strains and variants and corresponding permissive insect host cells that can be used for protein production are described in Luckow et al (1988), Miller et al (1986); Maeda et al (1985) and McKenna (1989).

### Methods for Producing Recombinant AAVs

The present disclosure provides materials and methods for producing recombinant AAVs in insect or mammalian cells. In some embodiments, the viral construct further comprises a promoter and a restriction site downstream of the promoter to allow insertion of a polynucleotide encoding one or more proteins of interest, wherein the promoter and the restriction site are located downstream of the 5′ AAV ITR and upstream of the 3′ AAV ITR. In some embodiments, the viral construct further comprises a posttranscriptional regulatory element downstream of the restriction site and upstream of the 3′ AAV ITR. In some embodiments, the viral construct further comprises a polynucleotide inserted at the restriction site and operably linked with the promoter, where the polynucleotide comprises the coding region of a protein of interest. As a skilled artisan will appreciate, any one of the AAV vector disclosed in the present application can be used in the method as the viral construct to produce the recombinant AAV.

In some embodiments, the helper functions are provided by one or more helper plasmids or helper viruses comprising adenoviral or baculoviral helper genes. Non-limiting examples of the adenoviral or baculoviral helper genes include, but are not limited to, E1A, E1B, E2A, E4 and VA, which can provide helper functions to AAV packaging.

Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US Publication No. 20110201088 (the disclosure of which is incorporated herein by reference), helper vectors pHELP (Applied Viromics). A skilled artisan will appreciate that any helper virus or helper plasmid of AAV that can provide adequate helper function to AAV can be used herein.

In some embodiments, the AAV cap genes are present in a plasmid. The plasmid can further comprise an AAV rep gene which may or may not correspond to the same serotype as the cap genes. The cap genes and/or rep gene from any AAV serotype (including, but not limited to, AAV1, AAV2, AAV4, AAV5, AAV6, AAV7, AAV8, AAV9, AAV10, AAV11, AAV12, AAV13 and any variants thereof) can be used herein to produce the recombinant AAV. In some embodiments, the AAV cap genes encode a capsid from serotype 1, serotype 2, serotype 4, serotype 5, serotype 6, serotype 7, serotype 8, serotype 9, serotype 10, serotype 11, serotype 12, serotype 13 or a variant thereof.

In some embodiments, the insect or mammalian cell can be transfected with the helper plasmid or helper virus, the viral construct and the plasmid encoding the AAV cap genes; and the recombinant AAV virus can be collected at various time points after co-transfection. For example, the recombinant AAV virus can be collected at about 12 hours, about 24 hours, about 36 hours, about 48 hours, about 72 hours, about 96 hours, about 120 hours, or a time between any of these two time points after the co-transfection.

Recombinant AAV can also be produced using any conventional methods known in the art suitable for producing infectious recombinant AAV. In some instances, a recombinant AAV can be produced by using an insect or mammalian cell that stably expresses some of the necessary components for AAV particle production. For example, a plasmid (or multiple plasmids) comprising AAV rep and cap genes, and a selectable marker, such as a neomycin resistance gene, can be integrated into the genome of the cell. The insect or mammalian cell can then be co-infected with a helper virus (e.g., adenovirus or baculovirus providing the helper functions) and the viral vector comprising the 5′ and 3′ AAV ITR (and the nucleotide sequence encoding the heterologous protein, if desired). The advantages of this method are that the cells are selectable and are suitable for large-scale production of the recombinant AAV. As another non-limiting example, adenovirus or baculovirus rather than plasmids can be used to introduce rep and cap genes into packaging cells. As yet another non-limiting example, both the viral vector containing the 5′ and 3′ AAV LTRs and the rep-cap genes can be stably integrated into the DNA of producer cells, and the helper functions can be provided by a wild-type adenovirus to produce the recombinant AAV.

### Cell Types Used in AAV Production

The viral particles comprising the disclosed AAV vectors may be produced using any invertebrate cell type which allows for production of AAV or biologic products and which can be maintained in culture. For example, the insect cell line used can be from Spodoptera frupperda, such as SF9, SF21, SF900+, drosophila cell lines, mosquito cell lines, e.g., Aedes albopictus derived cell lines, domestic silkworm cell lines, e.g. Bombyx mori cell lines, Trichoplusia ni cell lines such as High Five cells or Lepidoptera cell lines such as Ascalapha odorata cell lines. Preferred insect cells are cells from the insect species which are susceptible to baculovirus infection, including High Five, Sf9, Se301, SeIZD2109, SeUCR1, Sf9, Sf900+, Sf21, BTI-TN-5B1-4, MG-1, Tn368, HzAml, BM-N, Ha2302, Hz2E5 and Ao38.

Baculoviruses are enveloped DNA viruses of arthropods, two members of which are well known expression vectors for producing recombinant proteins in cell cultures. Baculoviruses have circular double-stranded genomes (80-200 kbp) which can be engineered to allow the delivery of large genomic content to specific cells. The viruses used as a vector are generally Autographa californica multicapsid nucleopolyhedrovirus (AcMNPV) or Bombyx mori (Bm-NPV) (Kato et al., 2010).

Baculoviruses are commonly used for the infection of insect cells for the expression of recombinant proteins. In particular, expression of heterologous genes in insects can be accomplished as described in for instance U.S. Pat. No. 4,745,051; Friesen et al (1986); EP 127,839; EP 155,476; Vlak et al (1988); Miller et al (1988); Carbonell et al (1988); Maeda et al (1985); Lebacq-Verheyden et al (1988); Smith et al (1985); Miyajima et al (1987); and Martin et al (1988). Numerous baculovirus strains and variants and corresponding permissive insect host cells that can be used for protein production are described in Luckow et al (1988), Miller et al (1986); Maeda et al (1985) and McKenna (1989).

In another aspect, the disclosed methods are carried out with any mammalian cell type which allows for replication of AAV or production of biologic products, and which can be maintained in culture. Preferred mammalian cells used can be HEK293, HeLa, CHO, NSO, SP2/0, PER.C6, Vero, RD, BHK, HT 1080, A549, Cos-7, ARPE-19, and MRC-5 cells.

### Recombinant Lentivirus Particles

The present disclosure provides recombinant viruses with lentiviral gene therapy vectors in combination with viral envelope proteins which enable transduction of hematopoietic stem cells, such as human CD34+ cells. In one embodiment, the disclosure provides a recombinant lentivirus composed of a lentivirus gene vector packaged in a heterologous envelope comprising the binding domain of a rhabdovirus envelope protein or an amino acid sequence derived therefrom. The disclosed lentiviral vector, at a minimum, lentivirus 5′ long terminal repeat (LTR) sequences. a molecule for delivery to the host cells, and a functional portion of the lentivirus 3′ LTR sequences. Optionally, the vector may further contain a w (psi) encapsidation sequence, Rev response element (RRE) sequences or sequences which provide equivalent or similar function. The heterologous molecule carried on the vector for delivery to a host cell may be any desired substance including, without limitation, a polypeptide, protein, enzyme, carbohydrate, chemical moiety, or nucleic acid molecule which may include oligonucleotides, RNA, DNA, and/or RNA/DNA hybrids. In one embodiment, the heterologous molecule is a nucleic acid molecule which introduces specific genetic modifications into human chromosomes, e.g., for correction of mutated genes. In another desirable embodiment, the heterologous molecule comprises a transgene comprising a nucleic acid sequence encoding a desired protein, peptide, polypeptide, enzyme, or another product and regulatory sequences directing transcription and/or translation of the encoded product in a host cell, and which enable expression of the encoded product in the host cell. Suitable products and regulatory sequences are discussed in more detail below. However, the selection of the heterologous molecule carried on the vector and delivered by the disclosed viruses is not a limitation of the present disclosure.

In selecting the lentiviral elements described herein for construction of the lentivirus vector and the recombinant virus, one may readily select sequences from any suitable lentivirus and any suitable lentivirus serotype or strain. Suitable lentiviruses include, for example, human immunodeficiency virus (HIV), simian immunodeficiency virus (SIV), caprine arthritis and encephalitis virus (CAEV), equine infectious anemia virus (EIAV), visna virus, and feline immunodeficiency virus (Hy), bovine immune deficiency virus (BIV). The examples provided herein illustrate the use of a vector derived from HIV. However, FIV and other lentiviruses of non-human origin may also be particularly desirable. The sequences used in the disclosed constructs may be derived from academic, non-profit (e.g., the American Type Culture Collection, Manassas, Va.) or commercial sources of lentiviruses. Alternatively, the sequences may be produced recombinantly, using genetic engineering techniques, or synthesized using conventional techniques (e.g., G. Barony and R. B. Merrifield, THE PEPTIDES: ANALYSIS, SYNTHESIS & BIOLOGY, Academic Press, pp. 3-285 (1980)) with reference to published viral sequences, including sequences contained in publicly accessible electronic databases.

The lentiviral vector contains a sufficient amount of lentiviral long terminal repeat (LTR) sequences to permit reverse transcription of the genome, to generate cDNA, and to permit expression of the RNA sequences present in the lentiviral vector. Suitably, these sequences include both the 5′ LTR sequences, which are located at the extreme 5′ end of the vector and the 3′ LTR sequences, which are located at the extreme 3′ end of the vector. These LTR sequences may be intact LTRs native to a selected lentivirus or a cross-reactive lentivirus, or more desirably, may be modified LTRs.

Various modifications to lentivirus LTRs have been described. One particularly desirable modification is a self-inactivating LTR, such as that described in H. Miyoshi et al, J. Virol., 72:8150-8157 (Oct. 1998) for HIV. In these HIV LTRs, the U3 region of the 5′ LTR is replaced with a strong heterologous promoter (e.g., CMV) and a deletion of 133 bp is made in the U3 region of the 3′ LTR. Thus, upon reverse transcription, the deletion of the 3′ LTR is transferred to the 5′ LTR, resulting in transcriptional inactivation of the LTR. The complete nucleotide sequence of HIV is known, see, L. Ratner et al. Nature. 313(6000):277-284 (1985). Yet another suitable modification involves a complete deletion in the U3 region, so that the 5′ LTR contains only a strong heterologous promoter, the R region, and the U5 region; and the 3′ LTR contains only the R region, which includes a polyA. In yet another embodiment, both the U3 and U5 regions of the 5′ LTRs are deleted and the 3′ LTRs contain only the R region. These and other suitable modifications may be readily engineered by one of skill in the art, in HIV and/or in comparable regions of another selected lentivirus.

Optionally, the lentiviral vector may contain a ψ (psi) packaging signal sequence downstream of the 5′ lentivirus LTR sequences. Optionally, one or more splice donor sites may be located between the LTR sequences and immediately upstream of the ψ sequence. According to the present disclosure, the ψ sequences may be modified to remove the overlap with the gag sequences and to improve packaging. For example, a stop codon may be inserted upstream of the gag coding sequence. Other suitable modifications to the ψ sequences may be engineered by one of skill in the art. Such modifications are not a limitation of the present disclosure.

In one suitable embodiment, the lentiviral vector contains lentiviral Rev responsive element (RRE) sequences located downstream of the LTR and ψ sequences. Suitably, the RRE sequences contain a minimum of about 275 to about 300 nt of the native lentiviral RRE sequences, and more preferably, at least about 400 to about 450 nt of the RRE sequences. Optionally, the RRE sequences may be substituted by another suitable element which assists in expression of gag/pol and its transportation to the cell nucleus. For example, other suitable sequences may include the CT element of the Manson-Pfizer virus, or the woodchuck hepatitis virus post-regulatory element (WPRE). Alternatively, the sequences encoding gag and gag/pol may be altered such that nuclear localization is modified without altering the amino acid sequences of the gag and gag/pol polypeptides. Suitable methods will be readily apparent to one of skill in the art.

Design of a transgene or another nucleic acid sequence that requires transcription, translation and/or expression to obtain the desired gene product in cells and hosts may include appropriate sequences that are operably linked to the coding sequences of interest to promote expression of the encoded product. “Operably linked” sequences include both expression control sequences that are contiguous with the nucleic acid sequences of interest and expression control sequences that act in trans or at a distance to control the nucleic acid sequences of interest.

Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. A great number of expression control sequences˜native, constitutive, inducible and/or tissue-specific—are known in the art and may be utilized to drive expression of the gene, depending upon the type of expression desired. For eukaryotic cells, expression control sequences typically include a promoter, an enhancer, such as one derived from an immunoglobulin gene, SV40, cytomegalovirus, etc. and a polyadenylation sequence which may include splice donor and acceptor sites. The polyadenylation (polyA) sequence generally is inserted following the transgene sequences and before the 3′ lentivirus LTR sequence. Most suitably, the lentiviral vector carrying the transgene or other molecule contains the polyA from the lentivirus providing the LTR sequences, e.g., HIV. However, other source of polyA may be readily selected for inclusion in the disclosed construct. In one embodiment, the bovine growth hormone polyA is selected. A lentiviral vector may also contain an intron, desirably located between the promoter/enhancer sequence and the transgene. One possible intron sequence is also derived from SV-40 and is referred to as the SV-40 T intron sequence. Another element that may be used in the vector is an internal ribosome entry site (IRES). An IRES sequence is used to produce more than one polypeptide from a single gene transcript. An IRES sequence would be used to produce a protein that contains more than one polypeptide chain. Selection of these and other common vector elements are conventional, and many such sequences are available (see, e.g., Sambrook et al. and references cited therein at, for example, pages 3.18-3.26 and 16.17-16.27 and Ausubel et al., CURRENT PROTOCOLS IN MOLECULAR BIOLOGY. John Wiley & Sons, New York, 1989).

In one embodiment, high-level constitutive expression will be desired. Examples of useful constitutive promoters include, without limitation, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EFlα promoter (Invitrogen). Inducible promoters, regulated by exogenously supplied compounds, are also useful and include, the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA. 93:3346-3351 (1996)), the tetracycline-repressible system (Gossen et al. Proc. Natl. Acad. Sci. USA, 89:5547-5551 (1992)), the tetracycline-inducible system (Gossen et al, Science. 268: 1766-1769 (1995), see also Harvey et al, Curr. Opin. Chem. Biol. 2:512-518 (1998)), the RU486-inducible system (Wang et al. Nat. Biotech. 15:239-243 (1997) and Wang et al, Gene Ther. 4:432-441 (1997)) and the rapamycin-inducible system (Magari et al, J. Clin. Invest. 100:2865-2872 (1997)). Other types of inducible promoters which may be useful in this context are those which are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally or in a tissue-specific manner, or in response to specific transcriptional stimuli. In a further embodiment, other native expression control elements, such as enhancer elements, polyadenylation sites or Kozak consensus sequences may also be used to mimic the native expression. Another embodiment of the transgene includes a transgene operably linked to a tissue-specific promoter.

Not all expression control sequences will function equally well to express all possible transgenes. However, one of skill in the art may make a selection among these expression control sequences without departing from the scope of this disclosure. Suitable promoter/enhancer sequences may be selected by one of skill in the art using the guidance provided by this application. Such selection is a routine matter and is not a limitation of the molecule or construct. For instance, one may select one or more expression control sequences may be operably linked to the coding sequence of interest, and inserted into the transgene, the vector, and the disclosed recombinant virus. After following one of the methods for packaging the lentivirus vector taught in this specification, or as taught in the art, one may infect suitable cells in vitro or in vivo. The number of copies of the vector in the cell may be monitored by Southern blotting or quantitative PCR. The level of RNA expression may be monitored by Northern blotting or quantitative RT-PCR. The level of expression may be monitored by Western blotting, immunohistochemistry, ELISA, MA or tests of the gene product's biological activity. Thus, one may easily assay whether a particular expression control sequence is suitable for a specific produced encoded by the transgene, and choose the expression control sequence most appropriate. Alternatively, where the molecule for delivery does not require expression, e.g., a carbohydrate, polypeptide, peptide, etc., the expression control sequences need not form part of the lentiviral vector or other molecule.

Optionally, the lentivirus vector may contain other lentiviral elements, such as those well known in the art, many of which are described below in connection with the lentiviral packaging sequences. However, notably, the lentivirus vector lacks the ability to assemble lentiviral envelope protein. Such a lentivirus vector may contain a portion of the envelope sequences corresponding to the RRE but lack the other envelope sequences. However, more desirably, the lentivirus vector lacks the sequences encoding any functional lentiviral envelope protein in order to substantially eliminate the possibility of a recombination event which results in replication competent virus.

Thus, the disclosed lentiviral vector contains, at a minimum, lentivirus 5′ long terminal repeat (LTR) sequences, (optionally) a ψ (psi) encapsidation sequence, a molecule for delivery to the host cells, and a functional portion of the lentivirus 3′ LTR sequences. Desirably, the vector further contains RRE sequences or their functional equivalent. Suitably, a lentiviral vector is delivered to a host cell for packaging into a virus by any suitable means, e.g., by transfection of the “naked” DNA molecule comprising the lentiviral vector or by a vector which may contain other lentiviral and regulatory elements described above, as well as any other elements commonly found on vectors. A “vector” can be any suitable vehicle which is capable of delivering the sequences or molecules carried thereon to a cell. For example, the vector may be readily selected from among, without limitation, a plasmid, phage, transposon, cosmid, virus, etc. Plasmids are particularly desirable for use in the disclosed methods of producing lentivirus. The selected vector may be delivered by any suitable method, including transfection, electroporation, liposome delivery, membrane fusion techniques, high velocity DNA-coated pellets, viral infection and protoplast fusion. According to the present disclosure, the lentiviral vector is packaged in a heterologous (i.e., non-lentiviral) envelope using the methods described below to form the recombinant virus.

### LV Envelope Protein

The envelope in which the lentiviral vector is packaged is suitably free of lentiviral envelope protein and comprises the binding domain of at least one heterologous envelope protein. In one embodiment, the envelope may be derived entirely from rhabdovirus glycoprotein or may contain a fragment of the rhabdovirus envelope (a rhabdovirus polypeptide or peptide) which contains the binding domain fused in frame to an envelope protein, polypeptide, or peptide, of a second virus. In an alternative, the envelope may contain a viral envelope protein comprising a sequence derived from the CD34+ cell transduction determinant discussed below. In another embodiment, the envelope may be derived entirely from arenavirus glycoprotein or a fragment thereof.

The rhabdovirus which provides the sequences encoding the envelope protein or a polypeptide or peptide thereof (e.g., the binding domain) can be derived from any suitable serotype from the vesiculovirus subfamily, e.g. VSV-G (Indiana), Morreton, Maraba, Cocal, Alagoa, Carajas, VSV-G (Arizona), Isfahan, VSV-G (New Jersey), or Piry. The sequences encoding the envelope protein may be obtained by any suitable means, including application of genetic engineering techniques to a viral source, chemical synthesis techniques, recombinant production or combinations thereof. Suitable sources of the desired viral sequences are well known in the art, and include a variety of academic, non-profit, commercial sources, and from electronic databases. The methods by which the sequences are obtained is not a limitation of the present disclosure. In one desirable embodiment, the heterologous envelope sequences are derived from a 31 amino acid human CD34+ cell transduction determinant that is found in all envelope proteins that can mediate transduction of human CD34+ cells but is not found in those that do not mediate transduction of human CD34+ cells.

Thus, in one embodiment, the envelope protein is intact rhabdovirus glycoprotein. Alternatively, it may be desirable to utilize a fragment of the selected rhabdovirus which contains, at a minimum, the binding domain of the rhabdovirus envelope glycoprotein, which is located within a 31 amino acid human CD34+ cell transduction determinant. Suitably, this rhabdovirus protein fragment is fused, directly or indirectly, via a linker, to a second, non-lentiviral, envelope protein or fragment thereof. This fusion protein may be desirable to improve packaging, yield, and/or purification of the resulting envelope protein. The second, non-lentiviral envelope protein or fragment thereof contains, at a minimum, the membrane domain. In one desirable embodiment, a truncated fragment of the 31 amino acid human CD34+ cell transduction determinant is fused to a VSV-G envelope protein. Still other fusion (chimeric) proteins according to the present disclosure can be generated by one of skill in the art.

In another embodiment, the envelope protein is an intact arenavirus envelope protein or a fragment of the selected arenavirus envelope protein which contains, at a minimum, the binding domain of the arenavirus envelope glycoprotein. Suitably, this arenavirus protein fragment is fused, directly or indirectly, via a linker, to a second, non-lentiviral, envelope protein or fragment thereof. This fusion protein may be desirable to improve packaging, yield, and/or purification of the resulting envelope protein. The second, non-lentiviral envelope protein or fragment thereof contains, at a minimum, the membrane domain.

Protective neutralizing antibody immunity against the arenaviral envelope glycoprotein (GP) is minimal, meaning that infection results in minimal antibody-mediated protection against re-infection if any. This characteristic allows for repeated immunization with vectors comprising the arenavirus envelope protein. Pre-existing immunity for arenavirus is low or negligible in the human population. In addition, arenavirus are generally non-cytolytic (not cell-destroying), and may under certain conditions, maintain long-term antigen expression in animals without eliciting disease.

Arenavirus envelope proteins may be from Lassa virus. Luna virus, Lujo virus, Lymphocytic choriomeningitis virus (LCMV), Mobala virus, Mopeia virus, Ippy virus, Amapari virus, Flexal virus, Guanarito virus, Junin virus, Latino virus, Machupo virus, Oliveros virus, Parana virus, Pichinde virus, Pirital virus, Sabia virus, Tacaribe virus, Tamiami virus, Bear Canyon virus, Whitewater Arroyo virus, Merino walk virus, Menekre virus, Morogoro virus, Gbagroube virus, Kodoko virus, Lemniscomys virus, Mus minutoides virus, Lunk virus, Giaro virus, and Wenzhou virus, Patawa virus, Pampa virus, Tonto Creek virus, Allpahuayo virus, Catarina virus, Skinner Tank virus, Real de Catorce virus, Big Brushy Tank virus, Catarina virus, and Ocozocoautla de Espinosa virus.

### Methods of Producing Recombinant Lentivirus

The recombinant lentivirus is replication defective, and therefore the virus is produced in a “producer cell line” in which the necessary constituents are provided in a single cell. As used herein, the term “producer cell line” refers to a cell line which is capable of producing recombinant retroviral particles, comprising a packaging cell line and a transfer vector construct comprising a packaging signal. The production of infectious viral particles and viral stock solutions may be carried out using conventional techniques. Methods of preparing viral stock solutions are known in the art and are illustrated by, e.g., Y. Soneoka et al. (1995) Nucl. Acids Res. 23:628-633, and N. R. Landau et al. (1992) J. Virol. 66:5110-5113. Infectious virus particles may be collected from the packaging cells using conventional techniques. For example, the infectious particles can be collected by cell lysis, or collection of the supernatant of the cell culture, as is known in the art. Optionally, the collected virus particles may be purified if desired. Suitable purification techniques are well known to those skilled in the art.

Three or four separate plasmid systems are used to generate the producer cell line. The four plasmid system comprises three helper plasmids and one transfer vector plasmid. For example, the Gag-Pol expression cassette encodes structural proteins and enzymes. Another cassette encodes Rev, which is an accessory protein necessary for vector genome nuclear export. A third cassette encodes a heterologous envelope protein, such as a vesiculovirus or arenavirus envelope protein, that allows lentivirus particle entry into target cells. The transfer vector cassette encodes the vector genome itself, which carries signals for incorporation into particles and an internal promoter driving transgene expression. The transfer vector carries the heterologous transgene and is the only genetic material is transferred to the target cells, e.g. CD34+ cell. The three plasmid system comprises two helper plasmids coding for the gag-pol and the envelope functions and the transfer vector cassette. See Merten et al., Mol. Ther. Methods Clin. Dev. 3: 16017, 2016.

The multiple constituent expression cassettes are transiently or stably transfected in the producer cell. In one embodiment, the producer cell line in which the necessary constituents are continuously and constitutively produced. The producer cell may be HEK293 cells, HEK293T cells, 293FT, 293SF-3F6, SODk1 cells, CV-1 cells, COS-1 cells, HtTA-1 cells, STAR cells, RD-MolPack cells, Win-Pac, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRCS cells, A549 cells, HT1080 cells, B-50 cells, 3T3 cells, NIH3T3 cells, HepG2 cells, Saos-2 cells, Huh7 cells, HeLa cells, W163 cells, 211 cells, and 211A cells. There are commercially available lentivirus packaging systems, e.g. Lenti Suite Kit (Systems Biosciences, Palo Alto, Calif.), Lenti-X packaging system (Takara Bio, Mountain View, Calif.), ViraSafe Packaging System (Cell Biolabs, Inc. San Diego, Calif.), ViroPower Lentiviarl Packaging Mix (Invitrogen) and Mission Lentiviral Packaging mix (Millapore Sigma, Burlington, Mass.).

In another embodiment, producer cell lines comprise inducible expression cassettes to express the packaging function. For example, the tetracycline-inducible expression system is used to generate the producer cells including the TET-Off system and the TET-On system. In addition, the ecdysone-inducible system is used.

Lentivirus production is performed using surface adherent cells grown in Petri dishes, T-flasks, multitray systems (Cell Factories, Cell Stacks), or HYPERFlask. At optimal confluence (<50%), cells are transfected using either the traditional Ca-phosphate protocol or the more recently developed polyethylenimine (PEI) method. Other efficient cationic transfection agents that are used include lipofectamine (Thermo-Fisher), fugene (Promega) LV-MAX (Thermo-Fisher), TransIT (Mirus) or 293fectin (Thermo-Fisher).

Alternatively, lentivirus production is performed using suspension cultures using shaker flasks, glass bioreactors, stainless steel bioreactor, wave bags, and disposable stirred tanks. The suspension cultures are transfected using Ca-phosphate or cationic polymers, and linear polyethyleneimine. The cells are also transfected using electroporation.

Purification of the lentivirus is carried out using membrane process steps such as filtration/clarification, concentration/diafiltration using tangential flow filtration (TFF) or membrane-based chromatography, and/or chromatography process steps such as ion-exchange chromatography (IEX), affinity chromatography, and size exclusion chromatography-based process steps. Any combination of these processes is used to purify the lentivirus. A benzonase/DNase treatment for the degradation of contaminating DNA is either part of the downstream protocol or is performed during vector production.

Purification is carried out three phases: (i) capture is the initial purification of the target molecule from either crude or clarified cell culture and leads to elimination of major contaminants. (ii) intermediate purification consists of steps performed on clarified feed between capture and polishing stages which results in removing specific impurities (proteins, DNA, and endotoxins), (iii) polishing is the final step aiming at removing trace contaminants and impurities leaving an active and safe product in a form suitable for formulation or packaging. Contaminants are often conformer to the target molecule, trace amounts of other impurities or suspected leakage products. Any type of chromatography and ultrafiltration process are used for the intermediate purification and the final polishing step(s).

Exemplary standard processes for purification of lentivirus include i) for removal of removal of cells and debris carried out with frontal filtration (0.45 μm) or centrifugation, ii) capture chromatography is carried out with anion-exchange chromatography such as Mustang Q or DEAE Sepharose, or affinity chromatography (heparin), iii) polishing is carried out with size-exclusion chromatography, iv) concentration and buffer exchange is carried out with tangential flow filtration or ultracentrifugation, v) DNA reduction is carried out with Benzonase and vi) sterilization is carried out with a 0.2-μm filter. See Merten et al., Mol. Ther Methods Clin Dev. 3: 16017, 2016.

Over the previous decade, multiple groups have evaluated the thermal stability of AAV capsids. While the size of the encapsulated genome has been shown to affect capsid thermal stability, some have since concluded otherwise. Importantly, these conflicting conclusions were drawn using different biophysical tools. Differential scanning fluorimetry (DSF) has commonly been used as a determinant of AAV capsid thermal stability. However, DSF solely measures the temperature at which the capsid proteins melt. While the melting of capsid proteins certainly delineates a disassembled capsid, it is possible for capsid structural integrity to be lost long before this melting event. Therefore, it can be argued this method is not an effective means of truly evaluating AAV capsid thermal stability. However, if the dye used in DSF (typically Sypro Orange) is exchanged with a dye which binds to DNA, such as SYBR Gold, capsid breakdown and DNA leakage can be evaluated rather than capsid protein melting. This method, in turn, offers a more accurate means of assessing capsid integrity. Using this method, rAAV5 capsids coating a range of single-stranded genome sizes were assessed and an inverse correlation between genome size and capsid thermal stability was found. Furthermore, these results were supported using a wide range of biophysical tools. Consequently, the present study concludes that the size of the encapsulated genome indeed plays a role in regulating AAV5 capsid thermal stability. Moving forward, this study highlights the necessity of using multiple biophysical tools to comprehensively characterization and elucidate structure-function of viral vectors used for gene therapy.

Other aspects and advantages of the present disclosure will be understood upon consideration of the following illustrative examples.

### EXAMPLES

### Example 1

### Comprehensive Characterization and Quantification of Adeno Associated Vectors by Size Exclusion Chromatography and Multi Angle Light Scattering

### Material and Methods

### Buffers

1.8× Phosphate Buffered Saline (PBS) with 10% EtOH was used as the mobile phase for isocratic chromatography in all SEC-MALS experiments. Stock solutions for this buffer were Dulbecco's PBS (10×) (Corning®, Corning, N.Y.), and 200-proof EtOH (Sigma-Aldrich, St. Louis, Mo.). Buffers were prepared with purified water from a Milli-Q® EMD Millipore system (Millipore, Burlington, Mass.) and filtered through a 0.2 μm polyether sulfone membrane (Nalgene, Rochester, N.Y.).

AAV5 H eavy and Light Capsids Production and Purification

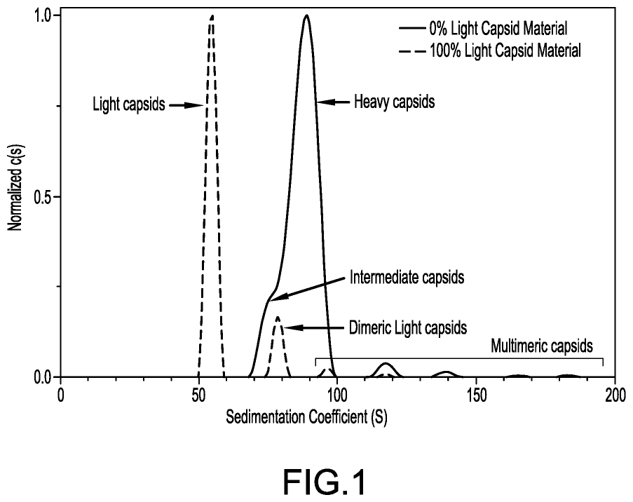

In this study, terms describing the extent of DNA packaging in capsid species, like “full”, “partially full,” and “empty”, are replaced by scientifically supportable “heavy”, “intermediate,” and “light” descriptors, respectively. This nomenclature is based on previous work showing purified AAV preparations to have a gradient of fullness based on the size of their encapsidated DNA and even “empty” capsids to not be completely devoid of DNA. (Torikai et al., J Virol 6, 363-369 (1970), de la Maza et al., J Biol Chem 255, 3194-3203 (1980), Lipps and Mayor, J Gen Virol 58 Pt 1, 63-72 (1982)). rAAV capsids referred to as Constructs 1 and 2, were generated from SF9 insect cell system by adapting and standardizing previously published method of baculo virus based production and purification process for AAV capsids. (Kohlbrenner et al., Mol Ther 12, 1217-1225 (2205), Smith et al., Mol Ther 17, 1888-1896 (2009)). As shown in FIG. 1, the final purified AAV capsid material contained 0% light capsids as analyzed by analytical ultracentrifugation. The light capsid material used for this study was a byproduct of capsid purification that was confirmed by analytical ultracentrifugation.

### AAV Heavy and Light Capsid Preparation

Total Cp and Vg titers of heavy and light capsid material were quantified by Capsid ELISA and qPCR using previously described methods. (Mayginnes et al., J Virol Methods 137, 193-204 (2006), Wang et al., Med Sci Monit Basic Res 19, 187-193 (2013)). Heavy and light capsids were diluted with proprietary phosphate-based buffer containing NaCl, pluronic acid, and sugar at pH 7.40 to a final concentration of 2.00e13 Cp/mL before analysis. All material was stored at −80° C. and thawed at room temperature (˜22-25° C.) prior to experiments. The materials were then combined by volume to generate a series of samples containing 0% to 100% light capsids at a final concentration of 2.00e13 Cp/mL for all samples.

### Size-Exclusion Chromatography

A Sepax SRT SEC-1000 column (4.6×300 mm) and guard column (Sepax, Newark, Del.) were used for all SEC-MALS experiments. The column was equilibrated with an isocratic mobile phase of PBS (2×)+10% EtOH at 0.2 mL/min for 12 hours. The flow rate was slowly ramped to 1 mL/min over 3 hours before loading 50 μL samples onto the column. The stationary and mobile phases were contained within an Agilent Series 1260 Infinity II LC System (Agilent, Waldbronn, Germany) consisting of an automated, thermally-controlled 1290 vial sampler at 4° C. and binary pump. UV absorbance of column eluates at 260 nm and 280 nm was detected by a multiple-wavelength diode array detector. ChemStation OpenLab LC systems software version 2.1.1.13 was used for controlling the HPLC system and analyzing UV absorbance data. All steps post-injection were performed at 25° C.

### Multi-Angle Light Scattering Analysis

A multi-angle light scattering (MALS) system was coupled downstream of the LC system. MALS signals were detected by a DAWN HELEOS 18-angle static light scattering (SLS) detector (Wyatt, Santa Barbara, Calif.) with a built-in QELS dynamic light scattering (DLS) detector and an Optilab rEX refractive index (RI) detector (Wyatt, Santa Barbara, Calif.). Astra 7.3.1 software was used for acquiring and analyzing UV, RI, and MALS data.

MALS uses the intensity of light scattered by molecules in solution to extricate the molar mass, size, and number of the light-scattering species. For each capsid species resolved in the flow mode, angular and concentration dependence of SLS intensity were measured by the detector and used by the Zimm equation (1) in ASTRA. (Wyatt et al., Analytica Chimica Acta 272, 1-40(1993)).

[Kc/R_{θ}]=((1/M)+2A₂c) {1+(16π²(R_{g})²/3λ²)sin² (θ/2)} (1)

where R_{θ} is the excess Raleigh ratio, c is the capsid concentration (mg/ml), θ is the scattering angle, M is the observed molar mass of each capsid particle, λ₂ is the second virial coefficient, λ is the wavelength of laser light in solution (658 nm), Rg is the radius of gyration of protein, and K is defined by Equation 2:

K=[4π²n² (dn/dc)²]/N₀λ⁴ (2)

where n is the refractive index of the solvent, do/dc is the refractive index increment of the capsids in solution, and N₀ is Avogadro's number (6.02×10²³ mol×⁻¹).

For a highly diluted capsid solution where (c→0), Equation 1 reduces to the straight-line equation (3):

[Kc/R_{θ}]=((1/M)+{1+((16π²(R_{g})²/3λ²)sin² (θ/2))} (3)

A plot of [Kc/R_{θ}] versus sin² (θ/2) will therefore yield a straight line that has a slope defined by 16π²(Rg)²/3Mλ² and y-intercept as 1/M. Equation 3 was used to derive the weighted-average molecular weight (M_{w}) and Rg for AAV capsids using a global analysis on the data acquired by 18 SLS detectors as defined by Equations 4 and 5:

Mw=Σ(cᵢMᵢ)/Σcᵢ (4)

Rg=Σ(cᵢRgᵢ)/Σcᵢ (5)

where ci is the protein concentration, Mᵢ is the observed molar mass, and Rgᵢ is the observed radius of gyration at the i-th slice within an elution profile.

The hydrodynamic radius (Rh) of each eluting species of AAV vectors was determined by the Wyatt QELS detector positioned at 90° with respect to the incident laser beam. Rh data is generated by measuring and iteratively fitting the time and concentration dependence of dynamic light scattering (DLS) intensity fluctuations using nonlinear least-squares regression analysis to the built-in equation (6) in ASTRA. The obtained Γ value from Equation 6 was used to calculate the translational diffusion coefficient (Dt) of each eluting capsid species using the built-in equation (7) which is finally used to calculate Rh by fitting into Stokes-Einstein equation (8). (Wang, Feng, et al., Medical science monitor basic research 19 (2013): 187, Koppel, J. Chem. Phys. 57, 4814-4820 (1972), Berne, Dynamic Light Scattering, Wiley, New York, N.Y. (1976))

G(τ)=α Exp(−2στ)+β (6)

Dt=[(Γλ₂)/(16π₂n² sin² θ/2)] (7)

Rh=[(k_{B}T)/(6πηDₜ)] (8)

G(τ) is the autocorrelation function of DLS intensity fluctuation I, α is the initial amplitude of the autocorrelation function at zero delay time, Γ is the decay rate constant of the autocorrelation function, τ is the delay time of the autocorrelation function, and β is the baseline offset (the value of the autocorrelation function at infinite delay time). λ is the wavelength of laser light in solution (658 nm), and n is the refractive index of the solvent, and θ is the scattering angle)(90° . Lastly, kB is Boltzmann's constant (1.38×10⁻²³ J K⁻¹), T is the absolute temperature, and η is the solvent viscosity.

The Rh reported here represents the weighted-average value as defined by Equation 9:

Rh=Σ(cᵢR_{h,i})/Σcᵢ (9)

where cᵢ is the protein concentration and R_{h,i} is the observed hydrodynamic radius at the i-th slice within an elution profile.

Capsid concentration (c) along the elution profile of each capsid species was automatically quantified in ASTRA from the change in refractive index (Δn) with respect to the solvent as measured by the Wyatt Optilab rEX detector using Equation 10:

c=(Δn)/(dn/dc) (10)

where dn/dc is the refractive index increment of the AAV vector in solution.

Since vectors are a combination of AAV capsid proteins and the encapsidated DNA, molar mass and concentration obtained directly from MALS represents the combined protein-DNA complex. To calculate the contribution of capsid proteins and encapsidated DNA separately, the built-in protein conjugate method in ASTRA was applied to all data sets. This method, adapted and further modified from M. Kunitani et.al, uses information from two different concentration detectors, RI and UV at 280 nm to determine the total concentration of the protein-DNA complex from the capsid protein using a system of equations. (Kunitani, Michael, et al., Journal of Chromatography A 588.1-2 (1991): 125-137, Chu Benjamin. Laser light scattering: basic principles and practice. Courier Corporation, 2007). The method works on the assumption that the RI (and UV) response is a sum of the responses from protein capsid and the encapsidated DNA. Equation 11 is used to calculate the combined dn/dc of the protein-DNA complex (V) as function of the mass fraction from the capsid protein (x):

$${( \frac{dn}{d c} )}_{V} = {( \frac{dn}{d c} )}_{CP} \cdot x + {( \frac{dn}{d c} )}_{DNA} \cdot ( 1 - x ) \qquad ( 11 )$$

where CP and DNA subscripts denote the intrinsic dn/dc values of 0.185 and 0.170 for the capsid protein and encapsidated DNA, respectively. Equation 12 is then used to calculate the concentration of the protein-DNA complex (C_{dRI}) based on the change in refractive index (Δn):

$${C}_{dRI} = \frac{Δ n}{{( \frac{dn}{d c} )}_{V}} \qquad ( 12 )$$

Similarly, equation 13 is used to calculate the combined extinction coefficient of the protein-DNA complex (εᵥ) as a function of the mass fraction from the capsid protein (x)

εᵥ=ε_{cp}·x+ε_{DNA}·(1−x) (13)

Where ε_{cp} and ε_{DNA} denote the intrinsic extinction coefficients of 1.790 mL/mg·cm and 17.000 mL/mg·cm for the capsid protein and encapsidated DNA, respectively. For the capsid protein, the coefficient was determined based on the VP proteins assuming their 1:1:10 ratio. Equation 14 is then used to calculate the concentration of the protein-DNA complex based on the A280 absorbance:

$${C}_{UV} = \frac{{A}_{280}}{{ε}_{υ} \cdot L} \qquad ( 13 )$$

Finally, because the concentration of the protein-DNA complex calculated by UV and RI are equal, Astra can then solve for the mass of the capsid protein using equation 15:

$$\frac{Δ n}{{( \frac{dn}{d c} )}_{cp} \cdot {x ( \frac{dn}{d c} )}_{DNA} \cdot ( 1 - x )} = \frac{{A}_{280}}{{ε}_{cp} \cdot x + {ε}_{DNA} \cdot ( 1 - x ) \cdot L} \qquad ( 15 )$$

Knowing the mass fraction from the capsid protein enables measuring physical attributes of the AAV capsid and encapsidated DNA independently. BSA (Thermo Scientific, Waltham, Mass.) [2 mg/mL] was used to normalize the light scattering detectors before AAV sample analysis.

### Analytical Ultracentrifugation

A Beckman Coulter ProteomeLab XL-I AUC (Beckman, Brea, Calif.) equipped with absorbance and Rayleigh interference (RI) optics was used for sample analysis. Samples were loaded into 2-sector sample cells containing Epon centerpieces. Cells were then loaded into an 8-hole rotor. Samples were temperature-equilibrated at 20° C. for no less than 2 hours. After temperature equilibration, sedimentation velocity centrifugation was performed on samples at 10,000 rpm for 10-12 hours and scans were collected at the maximum detection rate of the equipment.

Data were analyzed with the c(s) method as implemented in the program Sedfit and has previously been utilized for AAV capsid analysis. (Kunitani, Michael, et al., Journal of Chromatography A 588.1-2 (1991): 125-137, Schuck, Peter., Biophysical journal 78.3 (2000): 1606-1619). Briefly, Sedfit directly models the data with numerical solutions to the fundamental equation that describes diffusion and sedimentation in a sector shaped compartment, the Lamm equation (12):

∂c/∂t=[(∂²c/∂r)]−sω²[r(∂c/∂r)+2c] (12)

where c is total AAV concentration, t is time, D is diffusion constant, r is radius, s is sedimentation coefficient and ω is rotor speed. The two terms on the right side of the equation describe two competing forces: diffusion and sedimentation. The diffusion force is driven by molecular motion and moves toward a homogeneous solute solution. The sedimentation force is driven by the applied gravitational field and transports solute to the base of the cell.

### Data Analysis

GraphPad Prism version 8.2.1 was used to generate all data plots.

### Results

### AAV Characterization and Titer Estimation by Size Exclusion Chromatography

AAV samples were separated by SEC and the resulting elution profiles were monitored by a multi-detector system consisting of UV (260 and 280 nm), MALS, and RI detectors. The column effectively separated monomeric AAV capsid species (eluting ˜11.5 mins) from dimers (eluting ˜10.5 mins), higher order multimers (eluting <10 mins), and smaller nucleotide impurities and buffer components (eluting >12 min) (FIGS. 1A and 1B). By monitoring absorbance at 280 and 260 nm, each elution peak corresponding to different capsid species was characterized for its protein and DNA content based on its A260/A280 ratio. Monomeric heavy capsids had a consistent A260/A280 ratio of ˜1.34, while light capsids had a ratio of —0.6. DNA outside of intact capsids was detected in early elution peaks displaying A260/A280 ratios>1.7 (FIGS. 1A and 1B).

The Cp and Vg titers of denatured AAV2 capsids have previously been estimated using a UV-based bulk optical density method¹¹. Here, the SEC method was evaluated as a more advanced method for titer estimation which would not require highly purified or denatured capsids. AAV absorbance values at 280 and 260 nm were obtained via drop-line integration of the monomer and dimer peak areas in Chemstation. The A280 and A260 peak area measurements showed high reproducibility [CV_{A280}=0.36%, CV_{A260}=0.41%] as shown in Table A and were found to trend linearly with the amount of Cp and Vg loaded, respectively (Data not shown).

TABLE A

| | A280 Peak Area | A260 Peak Area |
|---|---|---|
| Average | 3377.77 | 2527.42 |
| Standard Deviation | 12.04 | 10.28 |
| CV (%) | 0.36 | 0.41 |

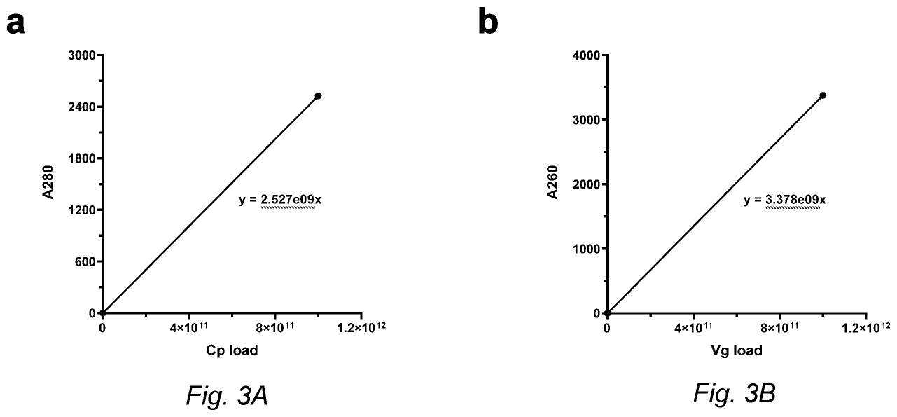

Due to the high reproducibility and linearity of the SEC assay, standard curves generated from AAV capsid material with known Cp and qPCR titers from ELISA and qPCR (FIGS. 3A and 3B) were used to calculate Cp and Vg titers for unknown samples. These standard curves, where y equals absorbance and x equals titer load, enable calculation of unknown titers from known absorbance values and the slope of the linear trendline. Specifically, calculating the Cp titer of an unknown sample simply requires dividing the A280 monomer and dimer peak area of the sample by the slope of the trendline (2.527e09, FIG. 3A) and multiplying by the injection volume (Equation 13).

$$Cp titer ( Cp / mL ) = ( \frac{A 280 peak area}{slope of A 280 linear trendline} ) * injection volume ( mL ) \qquad ( 13 )$$

Similarly, Vg titer is determined using the A260 peak area and slope (3.378e09, FIG. 3B) (Equation 14).

$$Vg titer ( Vg / mL ) = ( \frac{A 260 peak area}{slope of A 260 linear trendline} ) * injection volume ( mL ) \qquad ( 14 )$$

Using this method, the Cp and Vg titers of AAV samples containing 0-100% light capsids were calculated. While the SEC assay separates monomeric AAV capsids from higher- or lower-order impurities, it does not separate light from heavy capsids.

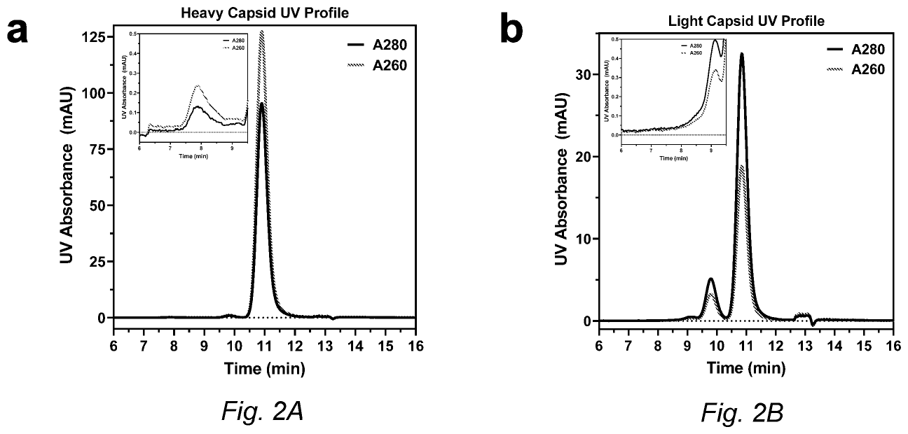

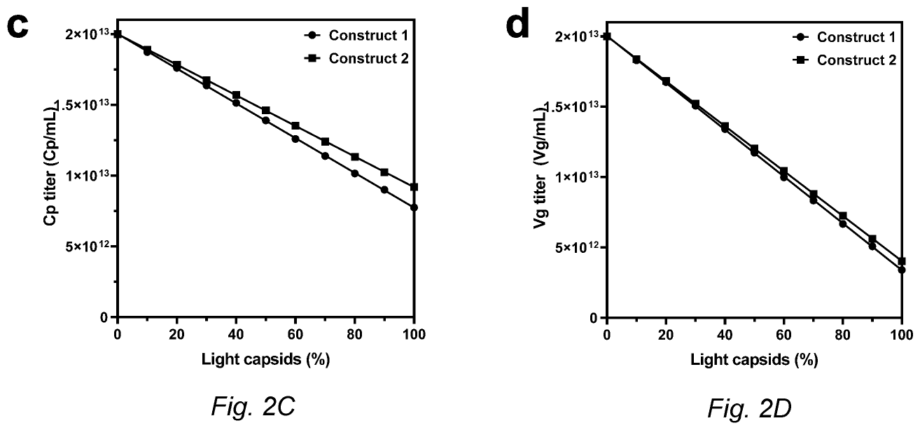

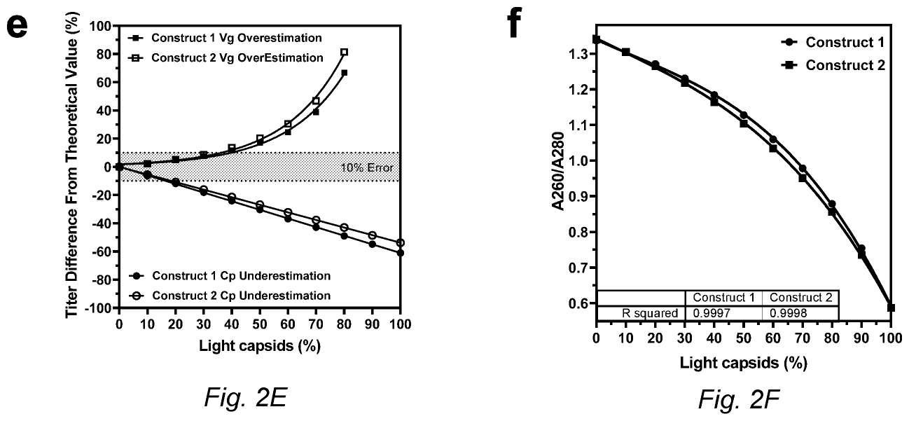

Consequently, linear decreases in both titers as a function of light-capsid content were observed, with R²>0.999 (FIGS. 2C and 2D). While a linear decline in Vg titer is expected with increasing light-capsids, the analogous drop in Cp titer indicates the influence of encapsidated vector genomes on A280 peak area. This genome contribution to the heavy capsid absorbance at 280 nm results in apparently higher Cp titers and highlights an erroneous assumption of the assay that the protein capsids and encapsidated DNA contribute exclusively to the A280 and A260 peak areas, respectively. Though this method in the current format is limited by A280 and A260 convolution, error in Cp and Vg titers of both constructs was less than 7% (underestimation) and 3% (overestimation), respectively, with samples containing up to 10% light capsids (FIG. 2E). 10% error in Cp and Vg titers was reached only with samples containing above 16% and 36% light capsids, respectively (FIG. 2E). With the high precision of the SEC assay taken into account, the error is still within the variability range of the widely-used PCR and ELISA titering methods in samples with even up to ˜50% light capsids. (Fagone et al., Hum Gene Ther Methods 23, 1-7 (2012), Kuck et al., J Virol Methods 140, 17-24 (2007), Dorange and Le Bec, Cell Gene Ther. Insights, 119-129 (2018). Further, using UV absorbance, the relative percentage of light to heavy capsids was estimated from A260/A280 peak area ratios. The A260/A280 ratios of AAV samples containing 0-100% light capsids were calculated and plotted as a function of light capsid content (FIG. 2F). The resulting plot best fit a third-order polynomial model. This polynomial regression model enables calculation of the light-capsid percentage of an AAV sample simply from its A260/A280 value. Although A260 and A280 convolution can be mitigated by applying a correction factor to titer calculations, we found that coupling MALS to SEC offers a more direct approach to circumvent this drawback as described below.

AAV Characterization and Titer Estimation Using Size-Exclusion Chromatography with Multi-Angle Light Scattering

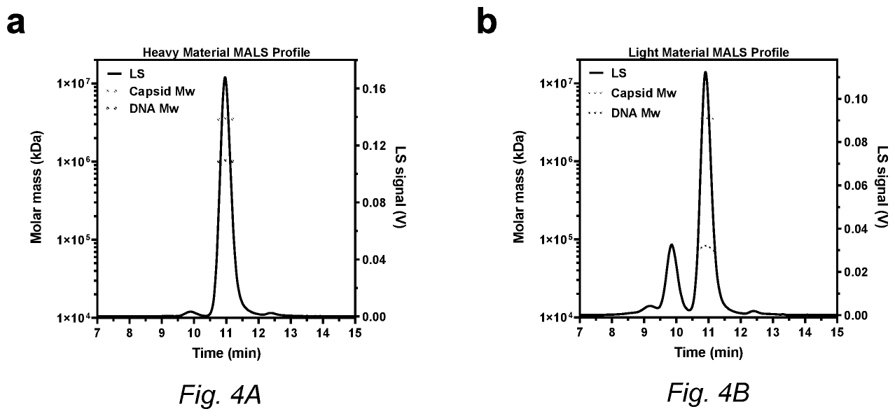

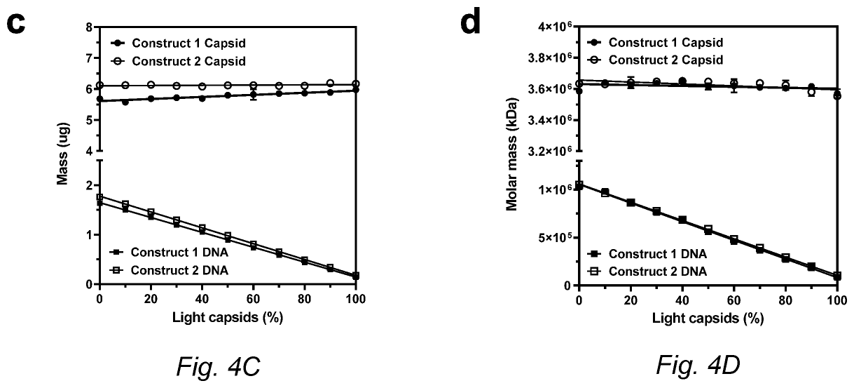

MALS has previously been coupled to SEC and other separation techniques to provide direct quantification and supplemental characterization of virus particles. (Koppel, J. Chem. Phys. 57, 4814-4820 (1972)). Unlike SEC, MALS is an absolute method and is not limited by A280 and A260 convolution. Briefly, MALS involves the detection of light scattered by species as a function of concentration and size in solution. ASTRA software then uses the angle of scattered light to quantify physical attributes of the scattering species. Using intrinsic properties of the protein and DNA, the Protein Conjugate Analysis feature in ASTRA calculates the mass and molar mass of the capsid and encapsidated DNA for heavy and light AAV samples (FIGS. 4A and 4B). Thus, a detailed summary of capsid integrity, aggregation, and physical features is achieved. Using the protein-conjugate feature, the capsid and encapsidated DNA mass and molar mass of AAV samples containing 0 to 100% light capsids were measured. As hypothesized, capsid mass was constant at around 6 micrograms (μg) while DNA mass decreased linearly from around 1.7 to 0.14 μg as a function of light capsid content, with R²>0.999 (FIG. 4C). Similarly, capsid molar mass remained consistent at around 3650 kilo Daltons (kDa) while the molar mass of the encapsidated DNA decreased linearly from around 1000 to 100 kDA with R²>0.997 (FIG. 4D). Mass and molar mass of capsid and encapsidated DNA, derived from MALSm were used to calculate Cp and Vg titers with Equations 15 and 16, where N_{A} is Avogadro's number (6.023e23).

$$Cp titer ( Cp / mL ) = \frac{capsid mass ( g )}{capsid molar mass ( \frac{g}{mol} )} \times \frac{{N}_{A} ( \frac{Cp}{mL} )}{injection volume ( mL )} \qquad ( 15 )$$

$$Vg titer ( Vg / mL ) = \frac{encapsidated DNA mass ( g )}{\begin{matrix}encapsidated DNA \\ molar mass ( \frac{g}{mol} )\end{matrix}} \times \frac{{N}_{A} ( \frac{Cp}{mL} )}{injection volume ( mL )} \qquad ( 16 )$$

Using these equations Cp and Vg titers of Construct 1 heavy capsid material was calculated to be 1.908e13 Cp/mL and 1.906e13 Vg/mL, respectively, with a Cp/Vg ratio of 1.00. These values are comparable to those previously obtained using Equations 13 and 14 (1.99e13 Cp/mL and 2.01e13 Vg/mL, respectively). While Equation 15 is independent of light capsid content, Equation 16 assumes that the AAV sample contains 0% light capsids. Consequently, calculating accurate Vg titers of samples with light and intermediate capsids requires accounting and correcting for relative capsid content.

### SEC MALS Estimation and Correction for Light and Intermediate Capsids

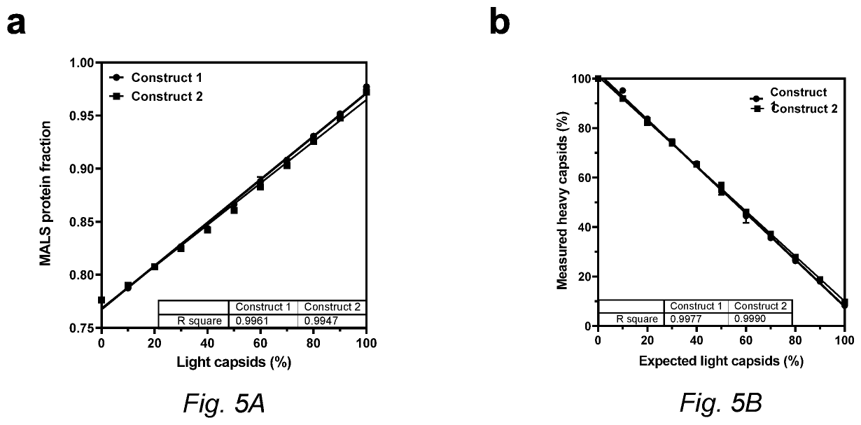

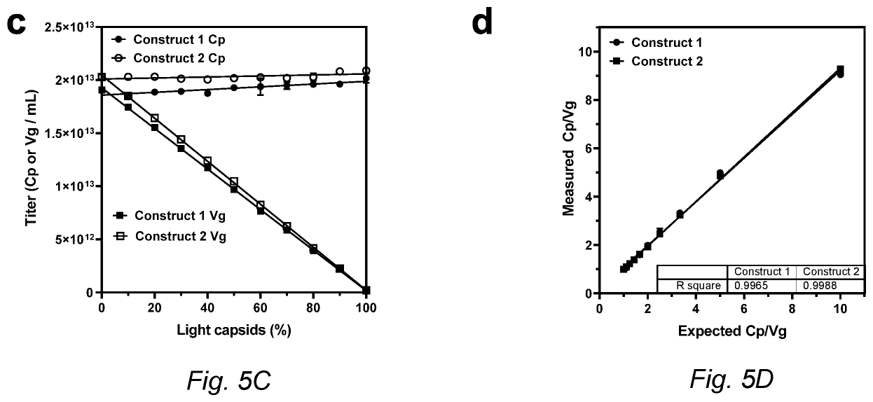

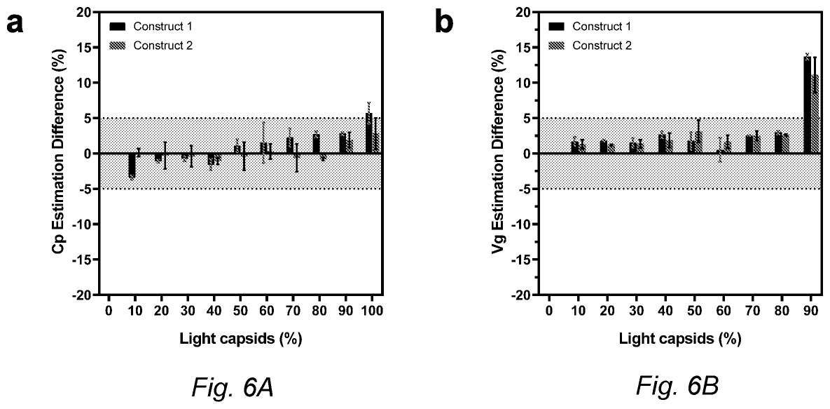

The coelution of light and heavy capsids from the SEC column necessitates determining their relative percentages to achieve accurate titers. SEC-MALS allows multiple ways to calculate relative capsid content. In addition to A260/A280 peak area, the MALS-derived protein fraction (relative capsid protein mass to protein DNA complex mass) enables estimation of light capsid content. The protein fraction trended linearly with light capsid content where 0% to 100% lights resulted in an increase of 0.77 to 0.98 (R²>0.99 for both constructs) (FIG. 5A) and can be used to correct for light capsid contribution to Vg titers. Even post-purification AAV vector preparations without light capsids do not exclusively contain heavy capsids. (Schuck, Peter. Biophysical journal 78.3 (2000): 1606-1619). AAV preparations are known to consist of capsids with varying-sized genomes that sediment in between heavy and light capsids when monitored by AUC (FIG. 1). The presence of these intermediate capsids results in the measured molar mass of the encapsidated DNA (1.03e06 kDa, FIG. 4D) being lower than the theoretical value (˜1.50e06 kDa). To account for light and intermediate capsids in SEC-MALS titer calculations, the packing efficiency of the capsids was determined by dividing the measured molar mass of the encapsidated DNA from a 0% light AAV5 sample by its theoretical value (Equation 17).

$$Packing efficiency = \frac{\begin{matrix}measured DNA molar mass of \\ 0 \% light ( \frac{g}{mol} )\end{matrix}}{\begin{matrix}theoretical DNA molar \\ mass ( \frac{g}{mol} )\end{matrix}} \qquad ( 17 )$$

Packing efficiency (PE) was then used to determine Heavy Capsid Ratio with Equation 18.

$$Heavy Capsid Ratio = \frac{measured DNA molar mass ( \frac{g}{mol} )}{\begin{matrix}theoretical DNA molar \\ mass ( \frac{g}{mol} ) \times PE\end{matrix}} \qquad ( 18 )$$

Using these equations, the heavy capsid content for AAV samples containing 0 to 100% light capsids was calculated. A plot of measured heavy capsid percentage as a function of known light capsid content fit a linear regression model with R²>0.99 (FIG. 5B). With the content of heavy capsids known, more accurate Vg titers were achieved by multiplying Cp titer by the ratio of heavy capsids. However, this calculation still assumes the light capsids in the sample do not have any genome. Since light capsids are not truly empty (FIGS. 4C and 4D), their encapsidated DNA skews Vg titer values. To prevent this, the Vg titer of a light AAV sample was calculated using Equation 19.

$$Vg titer of Light AAV Sample ( \frac{Vg}{mL} ) = Cp titer ( Cp / mL ) \times Heavy Capsid Ratio \qquad ( 19 )$$

The contribution of light capsid genomes to Vg titer values were then corrected with the known ratio of light capsids using Equation 20.

Light Capsid Ratio=1−Heavy Capsid Ratio (20)

Finally, putting these calculations together, Vg titer corrected to reflect relative heavy and light capsid content was achieved using Equation 21.

Corrected Vg Titer (Vg/mL)=(Cp titer*Heavy Capsid Ratio)−(Vg titer of Light AAV Sample*Light Capsid Ratio) (21)

MALS-derived Cp and corrected Vg titers were plotted as a function of light capsids for both constructs. While Cp values remained constant, Vg titers decreased linearly with increasing light capsid content (FIG. 5C). With corrected Vg titers, sample Cp/Vg ratios were calculated and plotted as a function of the expected values (FIG. 5D). The measured and expected Cp/Vg values demonstrated linear correlation with R²>0.99. The average difference in Cp and Vg titers from the expected values calculated for each sample is summarized in FIGS. 5A and 5B. In contrast to SEC-UV only derived titers, SEC-MALS improved titer accuracy, with less than 4% difference from expected values in samples spiked with up to 80% light capsids. Larger differences in Vg titer were observed in samples with 90-100% light capsids. These differences are likely due to difficulty in estimating the absolute extinction coefficient and dn/dc values of the light capsid genomes, which are variably sized. The calculations instead use the extinction coefficient and dn/dc values of the entire theoretical genome, which becomes less applicable for samples containing 90-100% light capsids.

In-Depth AAV Capsid Analysis with SEC-MALS

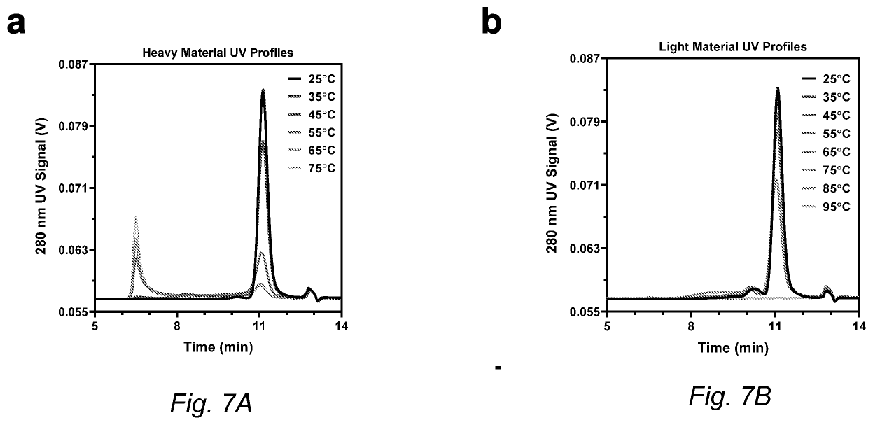

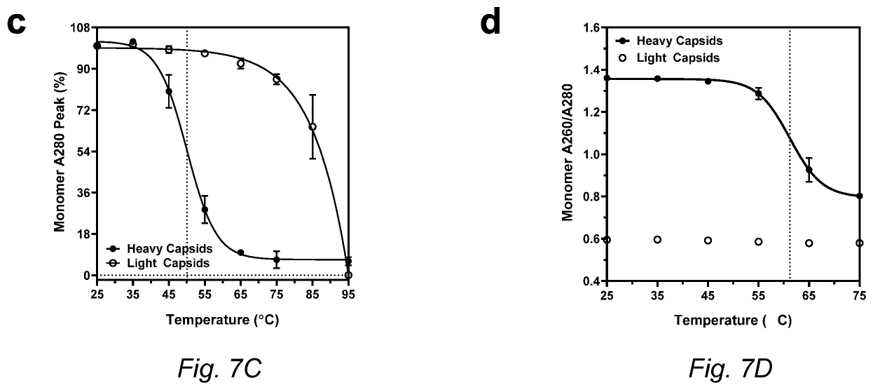

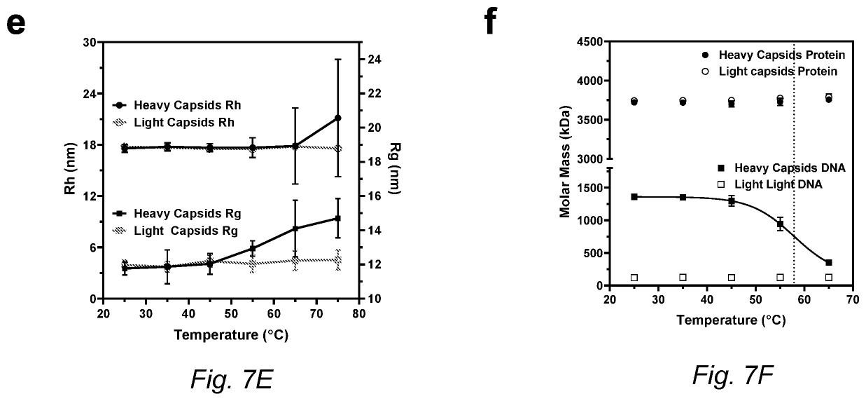

To demonstrate the practical applications of SEC-MALS, heavy and light AAV samples incubated at temperatures ranging from 25° C.-95° C. were analyzed. Samples were incubated for 30 minutes at each respective temperature directly prior to injection onto the SEC column. Various capsid forms (e.g. monomer, dimer, etc.) and extrinsic protein and nucleic acid impurities were observed using SEC A260 (data not shown) and A280 elution profiles for heavy material (FIG. 7A) and light material (FIG. 7B). Monomer peak area were also calculated for heavy and light capsids as a function of temperature (FIG. 7C). Changes in peak area correlated with biophysical changes in the sample which can be used to evaluate capsid integrity and stability. For heavy capsids, increasing temperature resulted in a decrease in the AAV monomer peak and an increase in the nucleic acid peak (A260/A280>1.7) at 6.5 minutes. The light capsids, meanwhile, were found to be much more thermally stable at higher temperatures, supporting the idea that internal pressure from the encapsidated DNA causes capsid instability. (Horowitz et al., J Virol 87, 2994-3002 (2013), Ivanovska et al., Proc Natl Acad Sci USA 104, 9603-9608 (2007)). Trends observed in both light and heavy capsid UV profiles were mirrored by the MALS elution profiles (data not shown). To evaluate whether the observed changes in the SEC elution profile represented capsid destabilization and genome release, the A260/A280 ratio as a function of temperature was monitored. At 25° C., the A260/A280 ratio of heavy capsids and light capsids was 1.34 and 0.6 respectively (FIG. 7D). As the temperature increased, the A260/A280 ratio for heavy capsids decreased to 0.8, with an inflection between 55 and 65° C., while that of light capsids remained constant. A decrease in the A260/280 ratio indicates a decrease in the amount of encapsidated DNA, which is further supported by the appearance of an increase free DNA peak at 3 min having A260/A280 ratio of ˜2 (FIG. 7A). To explore this further, MALS was used to monitor the size distribution and possible breakdown of capsids with temperature. The hydrodynamic radius (Rh) and radius of gyration (Rg) of the monomeric heavy and light capsid species were evaluated with increasing temperature. While the Rh and Rg of the light capsids remained constant, both radii were found to increase with increasing temperature for heavy capsids (FIG. 7E). An increase in both size and variability measured by MALS further supports the destabilization observed by A280 and A260/280. Interestingly, protein-Conjugate Analysis confirmed that the molar mass of the capsid protein remained constant for the heavy and light capsids, while the molar mass of the encapsidated DNA in heavy capsids decreased as a function of temperature (FIG. 7F). These results along with the observed extrinsic DNA by A280, support the event of a breakdown in capsid structure and DNA leakage above 45° C. Furthermore, they demonstrate the utility of SEC-MALS in elucidating biophysical changes in AAV, such as the increased thermal stability of light AAV capsids compared to heavy particles.

### Discussion

SEC-MALS is a simple, high-fidelity method to characterize wide-ranging physical attributes of AAV capsids. It provides a straightforward, single-method approach to measure AAV Cp and Vg titers without a standard curve and offers multiple ways to determine light to heavy capsid ratios. By exploiting the absorbance, light-scattering, and refractive properties inherent to the capsids and their encapsidated DNA, raw SEC-MALS data can be distilled into meaningful quantifications of capsid attributes. Its ease of use, reproducibility, and wealth of information it provides make SEC-MALS arguably one of the most versatile tools available for AAV characterization.

Cp and Vg titers of AAV capsids are commonly measured independently using Capsid ELISAs and qPCR, which can be time-intensive and highly variable highlighting the need for more accurate and precise titration methods. (Fagone et al., Hum Gene Ther Methods 23, 1-7 (2012), Kuck et al., J Virol Methods 140, 17-24 (2007), Dorange and Le Bec, Cell Gene Ther. Insights, 119-129 (2018). Vg titers reported using optical density have less variability than those of qPCR by up to 1-log. (Sommer et al., Mol Ther 7, 122-128(2003)). Though optical density is a simple assay capable of measuring both Cp and Vg titers, results can be skewed by protein and nucleic acid impurities. As another UV spectrophotometry method, SEC retains the advantages of optical density with the additional advantage of separating capsids from impurities on the column. Thus, AAV samples do not need to be highly purified to obtain accurate titers by SEC. Furthermore, inter-assay precision is substantially improved by SEC to <1%, compared to ˜16% in qPCR. (Lock et al., Hum Gene Ther Methods 25, 115-125 (2014), Paysic et al., Anal Bioanal Chem 408, 67-75 (2016), Pacouret et al., Mol Ther 25, 1375-1386 (2017)). A limitation of the SEC method is the coelution of light and heavy capsids from the column. As a result, titer error from A260 and A280 convolution increases with light-capsid content. However, the error in SEC Vg titers reaches to the routine variability of qPCR (˜15-20%) for samples containing over 50% light capsids only. These methods also share the common limitation of requiring a standard curve to calculate titers. While a correction factor for the presence of light capsids could be used to account for their absorbance contribution and improve the accuracy of SEC-derived titers that work is beyond the scope of the current study. The current study shows that combining SEC with MALS removes these limitations. MALS measurements are not affected by A260 and A280 convolution and, as an absolute method, MALS does not require a standard curve. ddPCR has also been shown to improve intra- and inter-assay precision to less than 2.21% and 8% as compared to 5.35% and 16.5% for qPCR, respectively, without a need for a standard curve. (Lock et al., Hum Gene Ther Methods 25, 115-125 (2014), Paysic et al., Anal Bioanal Chem 408, 67-75 (2016), Pacouret et al., Mol Ther 25, 1375-1386 (2017)). However, unlike SEC-MALS, the ddPCR method cannot measure both Cp and Vg titers along with other physical capsid attributes. SEC-MALS provides accurate Cp and Vg titers with improved precision in a 20-minute run without the need for sample manipulation, a standard curve, or a labor-intensive protocol. In addition, biophysical characteristics like capsid integrity and stability can also be monitored from the same method.

Somewhat of a swiss-army-knife method, SEC-MALS is a multifunctional approach to AAV characterization. It has emerged as a powerful tool for AAV product development and process analytics. This study highlights the potential of SEC-MALS for development and application to biophysically characterize viral vectors across industry and academic platforms.

### Example 2

### Improved Distribution Analysis and Quantification of Adeno-Associated Virus Particles by Size Exclusion Chromatography and/or Multi Angle Light Scattering (SEC-MALS) Techniques

### Material and Methods

### Sample Preparation

Analytical size exclusion chromatography: Sepax SEC 1000 column (7.8 mm ID×300 mm L) from Sepax technologies were used for analytical SEC analysis. The column utilizes Sepax proprietary surface technologies that achieves uniform hydrophilic, and neutral nanometer thick films chemically bonded on high purity and enhanced mechanical stability silica. The Sepax proprietary surface technologies allow the chemistry of thin film formation to be well controlled, which results in high column-to-column reproducibility. The nature of the chemical bonding and the maximum bonding density of the thin film benefit SRT SEC phases with high stability. The uniform surface coating enables high efficiency separation. The narrowly dispersed, spherical silica particles of the SRT packings for SEC-100, SEC-150, SEC300, SEC-500, SEC-1000 and SEC-2000 have nominal pore sizes at 100 Å, 150 Å, 300 Å, 500 Å, 1,000 Å, and 2,000 Å, respectively. Their specially designed large pore volume (ca. 1.35 mL/g for SRT SEC-150, 300 and 500, and ca. 1.0 mL/g for SRT SEC-100, 1000 and 2000) enables high separation capacity, leading to high separation resolution. SRT SEC columns are packed with a proprietary slurry technique to achieve uniform and stable packing bed density for maximum column efficiency.

50 μL samples were injected onto a Sepax SRT SEC-1000 column (referred to as the stationary phase) and eluted with an isocratic elution buffer (referred to as the mobile phase) of PBS (2×)+10% EtOH at a 1 mL/min flow rate. The stationary and mobile phases were contained within an Agilent Series 1260 Infinity II LC System (Agilent, Waldbronn, Germany) consisting of an automated, thermally-controlled 1290 vialsampler and binary pump. UV absorbance of column eluates at 260 nm and 280 nm was detected by a multiple-wavelength diode array detector, and ChemStation OpenLab LC systems software was used for controlling the HPLC system and analyzing UV absorbance data. Everything post-injection was performed at 22-25° C.

Multiangle light scattering (MALS): Multi Angle Light scattering analysis was performed using DAWN HELEOS 18-angle detector (Wyatt, Santa Barbara, Calif., USA) and an Optilab rEX refractive index detector (Wyatt, Santa Barbara, Calif., USA). Astra software was used for acquiring and analyzing MALS data.

Analytical ultracentrifugation (AUC): A Beckman Coulter ProteomeLab XL-I AUC equipped with absorbance and Rayleigh interference (RI) optics was used for analysis of all samples. Data were analyzed with the c(s) method as implemented in the program Sedfit. Within Sedfit, c(s) distributions were integrated to establish sedimentation coefficients of individual peaks, the signal average sedimentation coefficient and relative amounts of species.

Calculation of capsid and vector genome content: capsid and vector genome concentration was calculated based on following equations:

$$\frac{Cp}{ml} = \frac{Capsid Protein Mass ( g )}{Capsid Protein MW ( \frac{g}{mol} )} \times \frac{6.022 \times {10}^{23} ( \frac{cp}{mol} )}{injection vol ( mL )}$$

$$\frac{Vg}{ml} = \frac{\begin{matrix}Encapsidated DNA \\ Mass ( g )\end{matrix}}{Encapsidated DNA MW ( \frac{g}{mol} )} \times \frac{6.022 \times {10}^{23} ( \frac{cp}{mol} )}{injection vol ( mL )}$$

Protein and DNA mass is calculated by MALS and RI signals
Capsid protein and encapsidated DNA Mw is calculated by MALS and RI signal using following known parameters:

$$\frac{dn}{d c} ( AAV 5 Protein ) = 0.185$$

$$\frac{dn}{d c} ( Vector DNA ) = 0.17$$

$$ε 280 nm ( AAV 5 Capsid ) = 1.79$$

$$ε 280 nm ( Vector DNA ) = 17.$$

### Results

Characterizing and Quantifying AAV particles with SEC and SEC-MALS

SEC MALS System: The SEC-MALS system includes a HPLC system, a size exclusion column, UV detector, MALS detector, and a differential RI detector.

An example of a SEC system (i.e. a SEC-HPLC system) includes a size exclusion column fluidly connected to a source of a solvent and sample, a pump capable of flowing the solvent and sample through the size exclusion column, and an absorbance detector capable of measuring light absorption of the effluent from the size exclusion column.

An example of an SEC-MALS system, where the SEC-HPLC system with size exclusion column(s) is fluidly connected to the UV detector, MALS detector, and differential RI detector. In operation, a sample is first flowed through a SEC-HPLC system including size exclusion column(s). Effluent from the SEC-HPLC system is then flowed to the UV detector, MALS detector, and differential RI detector.

In using SEC or SEC-MALS to analyze a sample containing AAV particles, one can characterize properties of the AAV particles and quantify the titer of the AAV particles within a short time period (e.g. 20 minutes). Furthermore, both SEC and SEC-MALS are orthogonal techniques for multiple assays with minimum variability, provides process and long-term stability information fast and efficient in a high through put manner, and allows for different fractions to be collected and analyzed separately. Examples of the properties that can be characterized through SEC-MALS analysis include visualizing the aggregation profile of the AAV particles, estimating the concentration of nucleic acid that outside of or not encapsidated within intact capsids, examining the structural integrity of the capsids, estimating the percentage of AAV particles (i.e. heavy capsids) and empty capsids (i.e. light capsids) in the sample, determine particle size distribution (e.g. volume-based particle sizes or diameter/radius of the spheres) of the AAV particles, calculating the weight averaged molecular weights of the capsids and vector genomes. Examples of titer quantification include quantifying capsid titer and vector genome titer. To acquire the same information, a substantial number of conventional techniques such as analytical ultracentrifugation (AUC), electronic microscopy (EM), dynamic light scattering analysis (DLS), fluorescence analysis, enzyme-linked immunosorbent assays (ELISA), quantitative polymerase chain reaction (qPCR), and droplet digital PCR (ddPCR) would have to be employed.

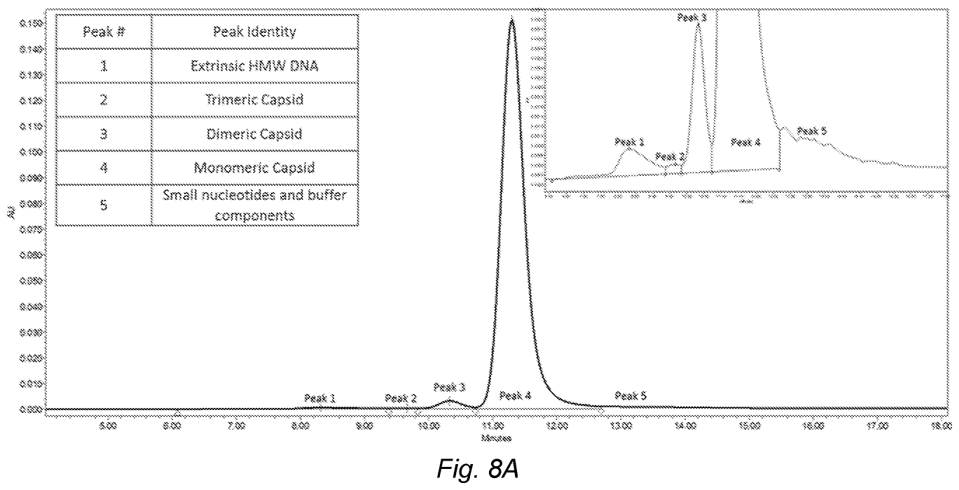

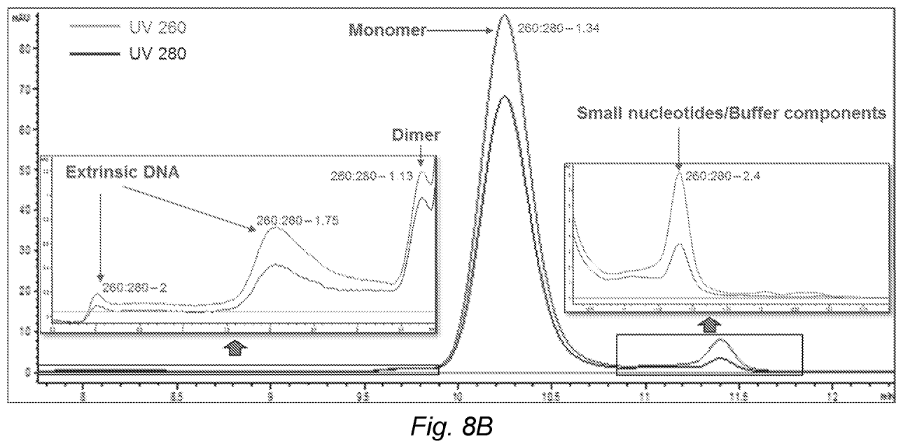

SEC-HPLC Analysis of AAV Preparation: SEC, as the name suggests, separates molecules in solution by size. The separation of AAV5 capsid particles using Sepax SRT SEC-1000 column was monitored by injecting 50 μL sample onto the column (referred to as the stationary phase) and eluted with an isocratic elution buffer (referred to as the mobile phase) of PBS (2×)+10% EtOH at a 1 mL/min flow rate. The stationary and mobile phases were contained within an Agilent Series 1260 Infinity II LC System (Agilent, Waldron, Germany) consisting of an automated, thermally-controlled 1290 vial sampler and binary pump. Column eluates were monitored using UV, MALS and RI detector. The resulting heterogeneity, distribution, and aggregation profile was captured by different detectors as shown in FIG. 8A.

FIG. 8A shows a representative SEC-HPLC profile of the sample measured at the 260 nm wavelength. The profile shows a number of absorbance unit (AU) peaks and each peak represents a different product or component. For example as shown in FIG. 8A, the main peak (i.e. peak #4) represents the monomeric capsids in the sample. Capsid aggregates such as trimeric and dimeric capsids are represented by peaks #2 and #3. Peak #1 represents high molecular weight nucleic acid from cells and peak #5 represents small nucleotides and buffer components.

Following sample elution from the column, UV absorbance at 260 and 280 nm is recorded to obtain elution profiles (FIG. 8A). A distinct main peak is eluted followed by smaller peaks on left side and right side of the main peak. These elution profiles feature a distinct major peak at ˜11.5 mins and minor peaks on left side and right side of the main peak. It is apparent that species in solution elute from the column in order of decreasing size, and AAVS monomeric capsid species are separated from higher order species and other extrinsic protein and DNA impurities. SEC column provides the ability to fraction collect the peaks separately for characterization. Each peak was isolated separately and characterized by various techniques like AUC, PCR and MALS to establish the identity of the eluting peaks. Data confirmed the presence of predominant monomeric capsid population encapsulating the gene of interest and further dimer and multimer forms of capsid along with extrinsic DNA and small nucleotide fragments as shown in FIG. 8A.

FIG. 8B shows another representative SEC-HPLC profile of the sample measured at the 260 nm and 280 nm wavelengths.

Monitoring elution profile at both 260 and 280 nm in this method provides a great tool to observe the presence of various forms of capsid (monomeric and higher aggregates) as well as any extrinsic protein or DNA based impurity in the sample (FIG. 8B). 260/280 ratio of monomeric capsid species was observed to be constant from batch to batch at ˜1.34 and other species like DNA or DNA protein complex was also found to be have a consistent ratio higher than the monomeric capsids.

The profile of FIG. 8B is similar to the profile of FIG. 8A in that the profile of FIG. 8B shows a main peak representing monomeric capsids. Additionally, the absorbance measurement at the 260 nm wavelength quantifies the nucleic acid concentration of the peaks and the absorbance measurement at the 280 nm wavelength quantifies the protein concentration of the peaks. By identifying the 260 nm/280 nm AU ratio, one could characterize the products represented by the peaks. For example, the main peak of FIG. 8B has a 260 nm/280 nm AU ratio of 1.34 and represents monomer AAV particles. FIG. 8B also shows aggregated AAV particles (e.g. dimer AAV particles) displaying a 260 nm/280 nm AU ratio of 1.13, extrinsic nucleic acid displaying 260 nm/280 nm AU ratios of 1.75 and 2, and small nucleotides and buffer components displaying a 260 nm/280 nm AU ratio of 2.4. Accordingly, monomer AAV particles can display a 260 nm/280 nm AU ratio ranging from greater than 1.13 to less than 1.75.

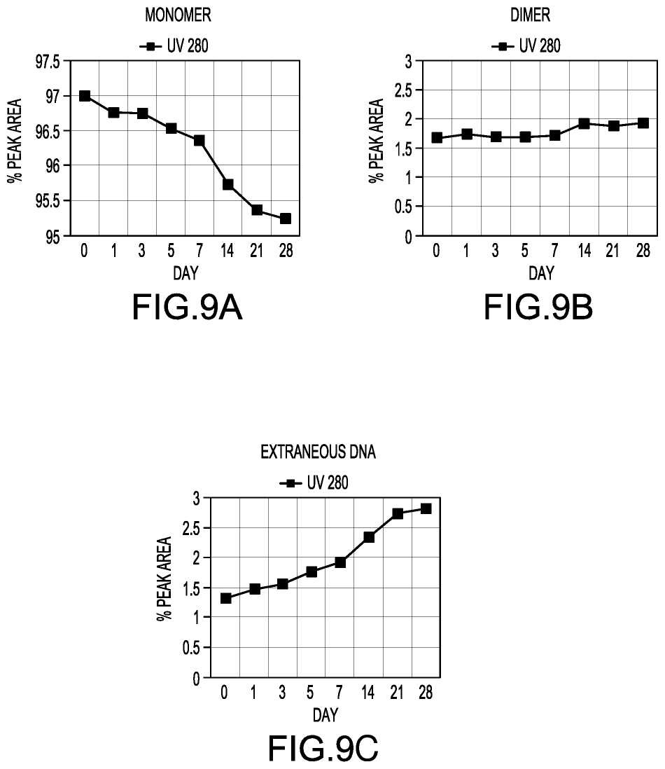

Analysis of Capsid Structural Integrity and Stability: FIGS. 9A, 9B, and 9C show an analysis of capsid stability of AAV samples, where each sample is modified to have a property that is different from the others.

The method can, for example, be utilized as a stability indicating assay since you can monitor the changes in the major peak areas as a function of time and keep a track of sample stability. A mini accelerated stability study was performed at 25° C. for 1 month to provide a proof of principal data to support the stability application of the method. The data showed that a two-fold increase in the extrinsic DNA peak and a corresponding drop in monomer peak area as function of time. Particularly, AAV samples were stored at were stored at 25° C. for 0, 1, 3, 5, 7, 10, 14, 21, and 28 days and separately analyzed by SEC-HPLC. The % peak area of the monomer AAV particles as shown in FIG. 9A decreased and the % peak area of extraneous nucleic acid as shown in FIG. 9C increased relative to longer storage time. Particularly, the % peak area of the extraneous deoxyribonucleic acid (DNA) peak increased linearly by about 2-fold. This suggests a change in the stability of the capsids. The % peak area of the dimer AAV particles as shown in FIG. 9B did not substantially change with longer storage times.

MALS Analysis of AAV Preparation: A sample from the AAV preparation was analyzed by MALS. As indicated above, effluent from the SEC-HPLC system is flowed to the UV detector, MALS detector, and differential RI detector.

The UV and differential RI detectors are used to provide the nucleic acid and protein concentration measurements.

The refractive index increments (dn/dc) for protein and nucleic acid (i.e. DNA) are used for calculating the concentration of protein and nucleic acid in the sample from measurements with the differential RI detector. The refractive index increments for protein and nucleic acid are provided below.

$$\frac{dn}{d c} ( Protein ) = 0.185$$

$$\frac{dn}{d c} ( DNA ) = 0.17$$

The extinction coefficients (E) for the capsid and vectors are used to calculate concentrations from absorbance measurements of the sample at 280 nm as measured by the UV detector. The extinction coefficients are derived from multiple absorbance measurements of empty capsids and unencapsulated vector genomes. For example, the extinction coefficients for an AAV5 capsid and vector genome are provided below.

ε(AAV5 Capsid)=1.79 ε(Vector DNA)=17.0

By using both refractive index and UV absorbance measurements, the mass of the capsids and vector genomes in the sample can be calculated.

The following equations apply to calculating the mass of the capsids and vector genomes from changes in the refractive index.

$${C}_{dRI} = \frac{Δ n}{{( \frac{dn}{d c} )}_{v}}$$

$${( \frac{dn}{d c} )}_{v} = {( \frac{dn}{d c} )}_{cp} \cdot x + {( \frac{dn}{d c} )}_{DNA} \cdot ( 1 - x )$$

Δn is the change in the refractive index as detected by the differential RI detector.

(dn/dc)ᵥ is the refractive index increment of the AAV vector.

(dn/dc)_{cp} is the refractive index increment of the capsid.

(dn/dc)_{DNA} is the refractive index increment of the vector genome.

x is the mass fraction of the capsid.

The following equations apply to calculating the mass of the capsids and vector genomes from changes in absorbance.

$${C}_{UV} = \frac{{A}_{280}}{{ε}_{v} \cdot L}$$

$${ε}_{v} = {ε}_{cp} \cdot x + {ε}_{DNA} \cdot ( 1 - x )$$

A₂₈₀ is absorbance as detected by the UV detector.

εᵥ is the extinction coefficient of the AAV vector.

L is the path length

ε_{cp} is the extinction coefficient of the capsid

ε_{DNA} is the extinction coefficient of the vector genome.

x is the mass fraction of the capsid.

As shown in the equations below, the calculations derived from the refractive index and UV absorbance correlate each other.

$${C}_{dRI} = {C}_{UV}$$

$$\frac{Δ n}{{( \frac{dn}{d c} )}_{cp} \cdot {x ( \frac{dn}{d c} )}_{DNA} \cdot ( 1 - x )} = \frac{{A}_{280}}{{ε}_{cp} \cdot x + {ε}_{DNA} \cdot ( 1 - x ) \cdot L}$$

The MALS detector employs static light scatter to measure the molar mass and concentration of the AAV particles. As shown in the equation below, light scattered at 0° is directly proportional to the molar mass and mass concentration of the AAV particles.

$${I ( θ )}_{scattered} ∝ M {c ( \frac{dn}{d c} )}^{2}$$

I(θ)_{scattered} is the intensity of the light scatter.

M is the molar mass of the AAV particles.

c is the concentration of the AAV particles.

do/dc is the refractive index increment of the AAV particles.

The MALS detector can also measure the average size of the particles by dynamic light scatter. With dynamic light scatter, the variation of scattered light with scattering angle is proportional to the average size of the scattering molecules. The average size of the particles includes measurements of the hydrodynamic radius (Rₕ) and radius of gyration (R_{g}). Rₕ is understood to be the radius of an equivalent hard sphere diffusing at the same rate as the molecule under observation. R_{g} is understood to be the mass weighted average distance from the core of a molecule to each mass element in the molecule.

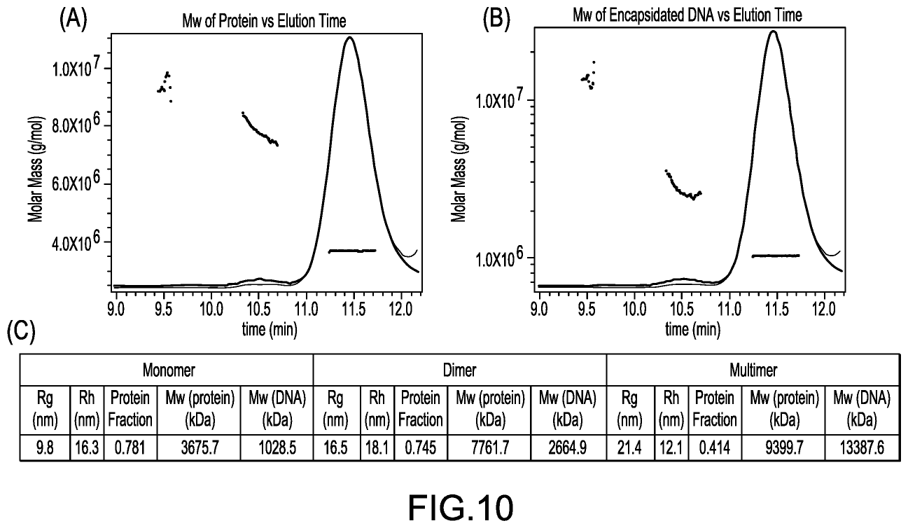

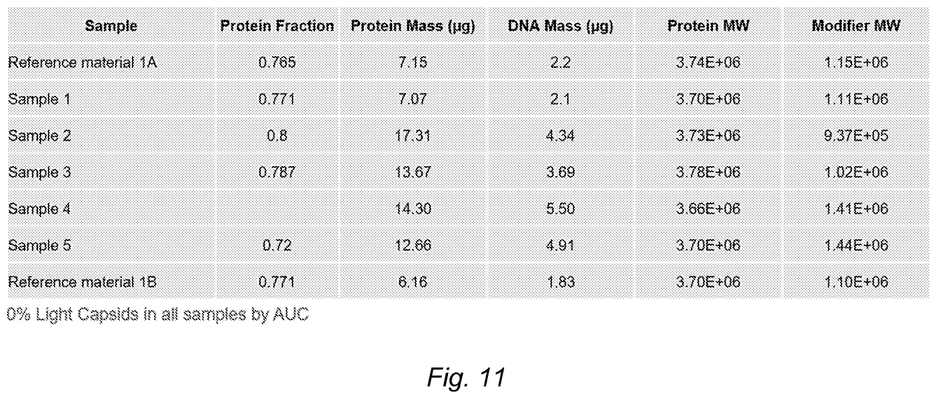

(A) and (B) of FIG. 10 show the molar mass of capsids and encapsidated DNA. The peaks shown in (A) and (B) of FIG. 10 correlate to the peaks shown in FIGS. 8A and 8B, where the major peak represent monomer AAV particles and the smaller peaks represent AAV particle aggregates. (A) and (B) of FIG. 10 also show that the particle size of the monomer AAV particles are substantially uniform. (C) of FIG. 10 and FIG. 11 show the particle sizes and molecular weights of the AAV particles. In addition to characterizing the properties of the AAV particles, the information is also useful for quantifying the titer of the AAV preparation. Also as indicated in FIG. 11, the percentage of light capsid in the sample was confirmed by analytical ultracentrifugation to be 0%.

Quantifying Titer of AAV in Preparation from SEC-MALS Analysis: Assuming that the sample does not contain empty capsids, the capsid and vector concentrations can be calculated using the following formulas.

$$Capsid Concentration ( cp / mL ) : \frac{Capsid Mass ( g )}{Capsid MW ( \frac{g}{mol} )} \times \frac{6.022 \times {10}^{23} ( \frac{cp}{mol} )}{injection vol ( mL )}$$

$$Vector Concentration ( vg / mL ) : \frac{DNA Mass ( g )}{DNA MW ( \frac{g}{mol} )} \times \frac{6.022 \times {10}^{23} ( \frac{cp}{mol} )}{injection vol ( mL )}$$

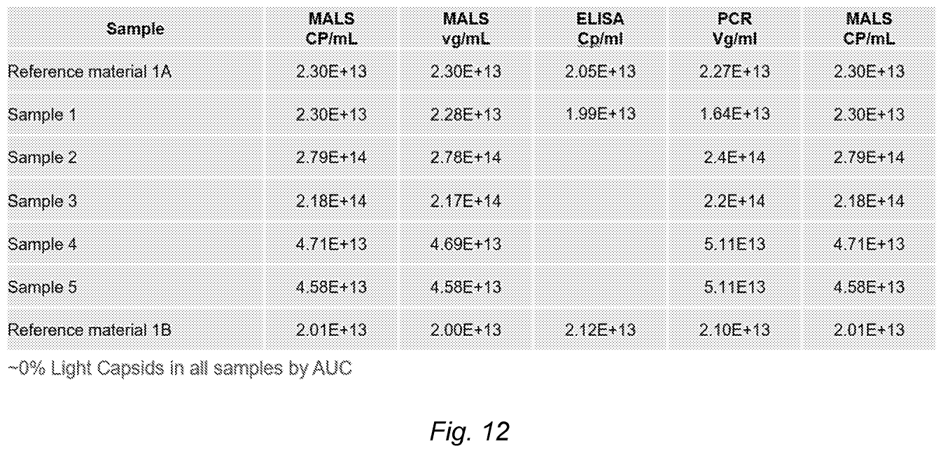

FIG. 12 shows the titer calculations from the MALS analysis as shown in FIG. 11. The percentage of light capsid in the sample was confirmed by analytical ultracentrifugation to be 0%.

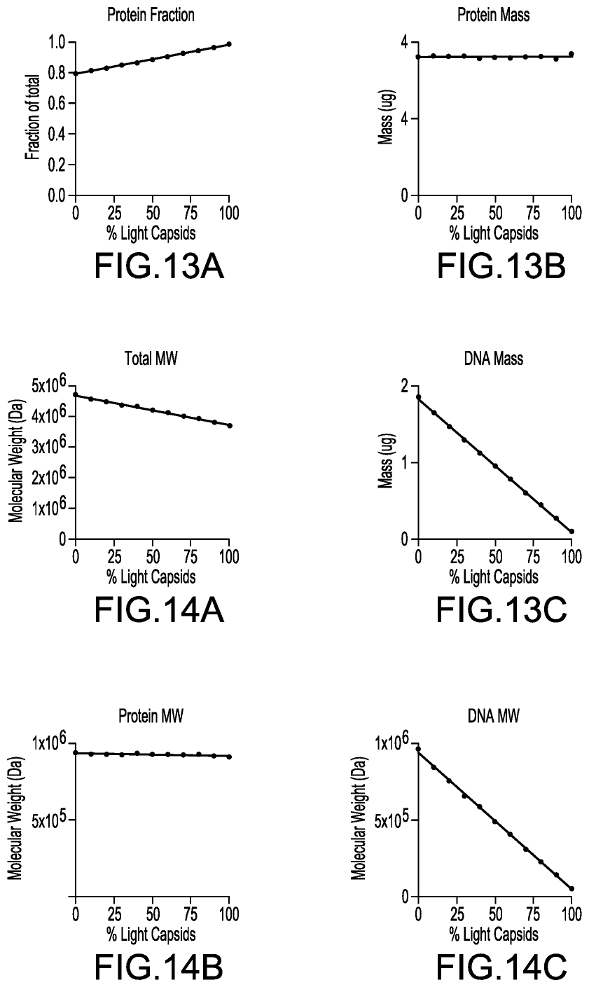

Quantifying Titer of AAV in Preparation Containing Empty Capsids: FIG. 13A, 13B, and 13C highlights that the total mass of nucleic acid in the sample decreases linearly with increasing concentrations of empty capsids. FIG. 13C particularly shows the total mass of nucleic acid fraction decreasing linearly with increasing concentrations of empty capsids, whereas FIGS. 13A and 13B show that the fraction of the protein increases and the total mass of the protein fraction stay the same with increasing the amount of empty capsid and reducing the amount of full capsids. Since the concentration of empty capsids effects the total mass of nucleic acid, the percentage of empty capsids should be determined in the starting material.

The weight of the vector genome combined with the weight of the capsid equals the total molecular weight of the AAV particle. For example, an AAV particle with a capsid MW of 3.73×10⁶ kilo Daltons (kDa) and a 4.9 kilobase (kb) vector genome MW of 1.53×10⁶ kDa has a theoretical MW of 5.23×10⁶ kDa. But as shown in FIG. 14A, the measured MW of the AAV particles is 4.71×10⁶ kDa. Also as shown in FIGS. 14B and 14C, the measured MWs for the capsid and 4.9 kb vector genome are 3.73×10⁶ kDA and 0.96×10⁶ kDa.

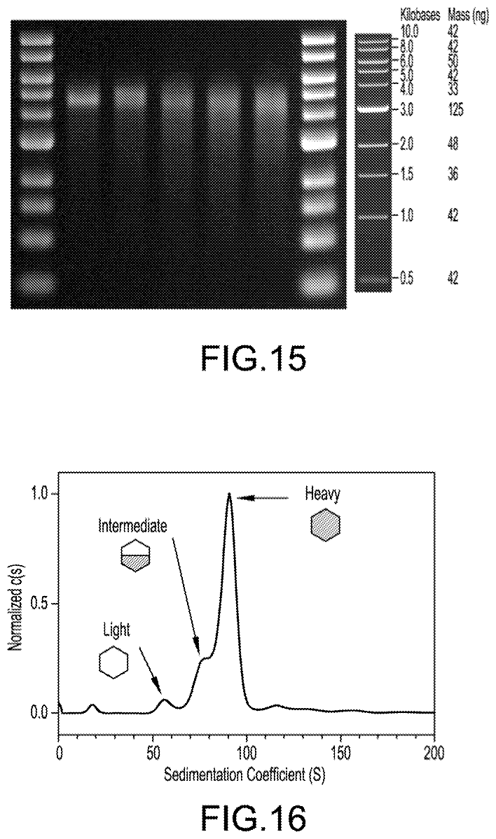

FIGS. 15 and 16 shows examples highlighting the samples have different concentrations of capsids with different MW vector genomes.

To address the discrepancy between the theoretical and calculated MW, one can look to the packaging efficiency of the AAV particle. For example, an equation is provided below to determine the packaging efficiency based upon the calculated MW of a 4.9 kb vector genome (i.e. 0.96 megadaltons (MDa) and theoretical packaging limit of an AAV particle (i.e. the theoretical MW of 4.7 kb vector genome or 1.44 MDa).

$$Packing Efficiency ( {PE}_{AAV 5} ) : \frac{Calculated {MW}_{DNA}}{Theoretical {MW}_{PL , DNA}} = \frac{0.96 E 6}{1.44 E 6} = 66 \%$$

Given the unexpected packaging efficiency determined by SEC-MALS, the following equations provides a correction for empty capsids in the sample.

$$\% Full Capsids ( PF ) = \frac{Measured {MW}_{DNA}}{Calculated {MW}_{PL , DNA}}$$

$$Calculated {MW}_{DNA} = Theoretical {MW}_{PL , DNA} \times PE$$

$$PF = \frac{Measured {MW}_{DNA} ( \frac{g}{mol} )}{Theoretical {MW}_{PL , DNA} \times {PE}_{AAV 5}}$$

The capsid and vector concentrations are calculated from the following equations.

$$Capsid Concentration ( cp / mL ) : \frac{Capsid Mass ( g )}{Capsid MW ( \frac{g}{mol} )} \times \frac{6.022 \times {10}^{23} ( \frac{cp}{mol} )}{injection vol ( mL )}$$

$$Vector Concentration ( vg / mL ) : \frac{DNA Mass ( g )}{DNA MW ( \frac{g}{mol} )} \times \frac{6.022 \times {10}^{23} ( \frac{cp}{mol} )}{injection vol ( mL )}$$

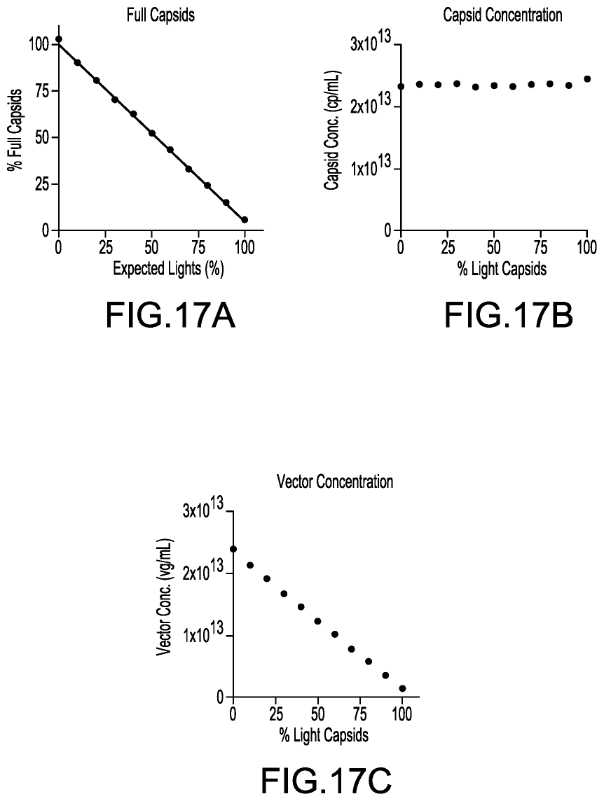

FIG. 17A highlights that the total concentration of full capsids (i.e. AAV particles) in the sample decreases linearly with increasing concentrations of empty capsids. FIG. 17C particularly shows that the vector genome concentration decreases linearly with increasing concentrations of empty capsids, whereas FIG. 17B shows that the capsid concentration stays the same with increasing concentrations of empty capsids.

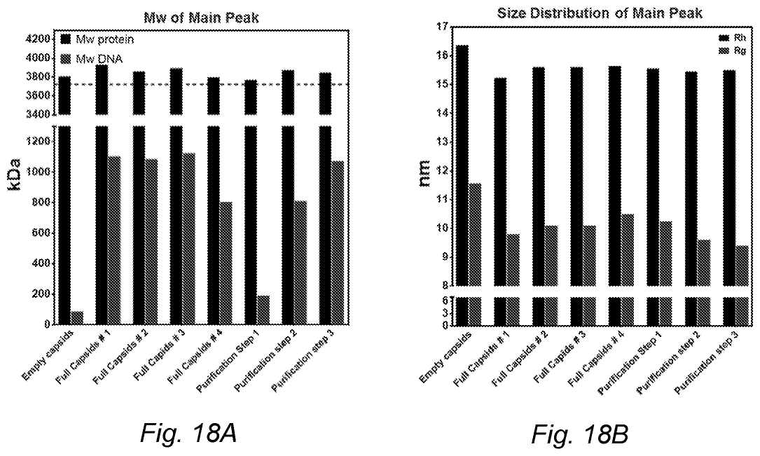

Analysis of MW of Size Distribution of Different Samples: FIG. 18A highlights that the MW of the capsids remained constant but MW of encapsidated vector genome changed as function of empty capsids. FIG. 18B highlights that the Rₕ of capsids is constant and that the R_{g} is dependent on percent concentration of empty capsid in the sample.

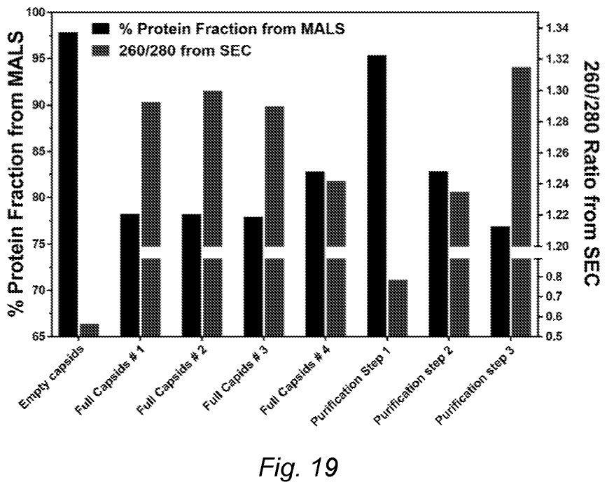

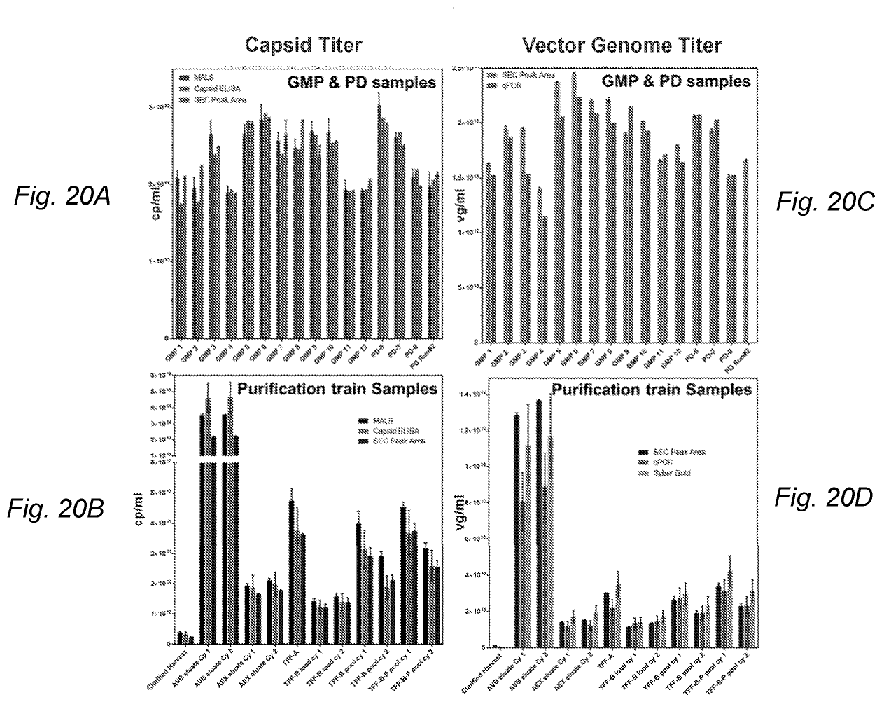

Based on these calculation, an estimation of the percent concentration of empty capsid can be determined as shown in FIG. 19. As shown in FIGS. 20A, 20B, 20C, and 20D, these estimations are comparable to conventional assays.

### Example 3

### Biophysical Stability Investigation of Viral Capsids Used for Gene Therapy

### Materials and Methods

All buffer components were purchased from J. T. Baker (Center Valley, Pa., USA). Buffers were prepared with purified water from a Milli-Q® EMD Millipore system (Burlington, Mass., USA) and filtered through a 0.2 μm polyether sulfone membrane (Nalgene, Rochester, N.Y., USA).

Viral Samples: In-house lentiviral (LV) pH study samples were dialyzed against citrate (pH 4.00), phosphate (pH 6.00 and pH 8.00), Tris (pH 7.40), or carbonate (pH 10.00) buffers containing 300 mM NaCl and 2 mM MgCl2, while lentiviral salt study samples were dialyzed against Tris buffer (pH 7.40) with 2 mM MgCl2 at the desired salt concentration (0-1 M). All dialyses were performed over a 15-minute period with 0.1 mL, 20 kDA molecular weight cut-off Slide-A-Lyzer™ MINI Dialysis Devices (Thermo Fisher Scientific, Waltham, Mass., USA). Though this method slightly diluted samples, the dialysis devices were not found to alter the size or structure of LV particles (data not shown). Adeno-associated viral (AAV) samples were obtained from internal process development runs, hereafter referred to as “Source A,” or acquired from ViGene Biosciences (Rockville, Md., USA), referred to as “Source B.”

LV Biophysical Characterization Method Development: For preliminary SEC experiments, 100 μL of LV samples were injected onto a TSKgel G5000PWXL column (300.0 mm×7.8 mm i.d.), with a 2× PBS, pH 7.40 buffer used for isocratic elution at a 0.300 mL/min flow rate. Elution fractions were collected automatically, using ChemStation OpenLab LC systems software, according to time, with each fraction collected over a 2-minute period from 17-41 minutes. Method optimization involved the evaluation of two columns, three flow rates, and nine buffers, summarized in Table B. The TSKgel G5000PWXL column used in preliminary experiments was selected with a Tris buffer mobile phase (20 mM Tris, 300 mM NaCl, 2 mM MgCl2, pH 7.40) at a flow rate of 0.300 mL/min. All experiments were performed from 22-25° C.

TABLE B
Summary of columns, flow rates, and elution buffers evaluated
for LV chromatography method optimization. Selected method
parameters are represented in bold.
Columns
TSKgel G5000PWXL
TSKgel G-DNA-PW
Flow Rates
0.300 mL/min
0.500 mL/min
1.000 mL/min
Buffers
2xPBS, pH 7.40
2xPBS, 10% EtOH, pH 7.40
20 mM Tris, 150 mM NaCl, pH 7.40
20 mM Tris, 150 mM NaCl, 2 mM MgCl₂, pH 7.40
20 mM Tris, 150 mM NaCl, 2 mM MgCl₂, 2% sucrose, pH 7.40
20 mM Tris, 300 mM NaCl, pH 7.40
20 mM Tris, 300 mM NaCl, 2 mM MgCl₂, pH 7.40
20 mM Tris, 300 mM NaCl, 2 mM MgCl₂, 2% sucrose, pH 7.40

Size-Exclusion Chromatography coupled to multi-angle light scattering (SEC-MALS): SE-HPLC experiments were performed on an Agilent Series 1260 Infinity II LC System (Agilent, Waldbronn, Germany) consisting of an automated, thermally-controlled 1290 vialsampler and binary pump. UV absorbance at 280 nm and 260 nm was detected by a multiple-wavelength diode array detector, and ChemStation OpenLab LC systems software was used for controlling the HPLC system and analyzing UV absorbance data. Coupled to our LC system, MALS signals were detected by a DAWN HELEOS 18-angle detector (Wyatt, Santa Barbara, Calif., USA) and an Optilab rEX refractive index detector (Wyatt, Santa Barbara, Calif., USA). Astra 7.1.2 software was used for acquiring and analyzing MALS data. For lentiviral experiments, 25 μL of sample was injected onto a TSKGel G5000PWXL column with a Tris buffer mobile phase at a flow rate of 0.3 mL/min (as described above). All samples were thawed from the freezer at 25° C. directly prior to injection. Everything post-injection was performed at 22-25° C. For adeno-associated viral particle experiments, 25 μL of sample was injected, 2× PBS+10% EtoH was used for isocratic elution, and a flow rate of 1 mL/min was applied. All samples were thawed from the freezer at 37° C. prior to injection. Everything post-injection was performed at 22-25° C.

Far UV Circular Dichroism (CD) Spectroscopy: Far UV CD spectra were collected using a Jasco J-1500 spectropolarimeter equipped with a six-position cuvette holder (Jasco, Oklahoma City, Okla., USA), thermostatically controlled at 25° C. For lentiviral experiments, 30 μL of sample was loaded in a 0.1 mm path length cuvette while 170 μL of sample was loaded in a 1 mm path length cuvette for adeno-associated viral experiments. All data were collected in the 190- to 250-nm wavelength range with a resolution of 0.2 nm, scanning speed of 50 nm/min, response time of 4 s, and a bandwidth of 2 nm. Spectra presented are an average of 10 consecutive measurements.

Dynamic Light Scattering (DLS): An UNcle all-in-one biologics stability screening platform (UNchained Labs, Pleasanton, Calif., USA) was used to record DLS measurements over the course of a stepped temperature-ramp designed under Freeform Mode. This freeform method was designed to achieve measurements in 2 degree increments from 25-95° C. in as little time as possible. 8.8 μL samples were loaded into Uni wells directly prior to measurement. 4 DLS measurements were taken at 5 seconds each for each sample. All samples were measured in triplicate.

Intrinsic Capsid Fluorescence: The “Tm & Tagg with optional DLS” application of the UNcle instrument was used to record intrinsic fluorescence of exposed tryptophan or tyrosine capsid-protein residues. Using laser-excited light at a wavelength of 473 nm, fluorescence emission spectra were recorded at wavelengths between 500 and 700 nm. 8.8 μL samples were loaded into Uni wells and heated at a stepped thermal ramp from 25 to 95° C. in 2.5° C. increments. All samples were measured in triplicate.

Extrinsic Capsid Fluorescence: The “Tm With SYPRO” application of the UNcle instrument was used to record sample extrinsic fluorescence. SYBR Gold Nucleic Acid dye (Invitrogen, Carlsbad, CA, USA) diluted in phosphate buffer to the recommended working concentration was added to each sample immediately prior to loading in the 8.8 μL Uni wells. The same excitation/emission wavelengths and temperature program used in the intrinsic fluorescence experiment were followed. All measurements were done in triplicate. The lowest unfolding temperature was determined fitting the fluorescence intensity as a function of temperature to the Boltzmann Equation. The transition temperature value corresponds to the mid-point or inflection point of the transition.

Alkaline Agarose Gel Electrophoresis: Encapsulated genome size distribution was confirmed by 0.8% agarose gel electrophoresis, staining with GelRed Nucleic Acid Gel Stain (Biotium), and visualizing under UV-light using a Chemidoc imaging system. Agarose gel was prepared dissolving 0.8 g SeaKem LE agarose in 100 mL 1× alkaline buffer (50 mM NaOH, 1 mM EDTA). Before electrophoresis, 1.0E+11 vg of samples was added to 1.5 μL 10% SDS (Ambion) and 7.5 μL 6× Alkaline Gel Loading Dye (Alfa Aesar) and brought to 25 with 1× alkaline buffer. 7.8 μL of 1 kb ladder (New England Biolabs) was added to 6.3 μL 10% SDS, 30 μL 6× alkaline loading dye, and 30.95 μL of 1× alkaline buffer for triplicate runs. 18 μL of samples and ladders were loaded on the gel, which was resolved using 52 V/cm for 3.5 hours, before three 10-minute washes in neutralizing buffer (1 M Tris-HCl, 1M NaCl, pH 7.40). DNA on the gel was stained with GelRed solution (30 μL GelRed, 20 mL neutralizing buffer, 180 mL Milli-Q H2O) for 30 minutes, followed by three 10-minute washes in Milli-Q H2O. DNA was visualized on a Chemidoc imaging system using the GelRed read setting.

### Results

### Qualitative and Quantitative Characterization of Lentivirus Particles by Size-Exclusion Chromatography Coupled to Multi-Angle Light Scattering

Using a method previously outlined in Steppert et al. (J. Chromatogr. 1487: 89-99, 2017) for the characterization of virus-like particles as a starting point, a SEC-MALS method to biophysically analyze LV particles was developed. This method allowed for qualitative and quantitative evaluation of LV particles, and offered a new, reproducible way to rapidly estimate LV particle titer.

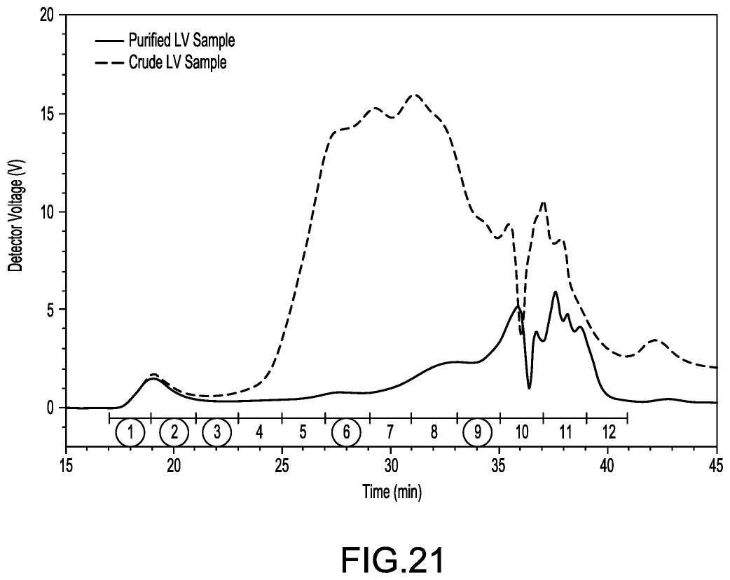

Preliminary SEC experiments were performed by injecting 100 μL of both crude and purified LV samples onto the TSKgel G5000PWXL column (FIG. 21). 2× PBS at pH 7.40 was used as an elution buffer and a flow rate of 0.3mL/min was applied. SEC elution profiles, detected by UV absorbance at 280 nm. Fractions 1-12 were collected after injection of purified LV sample and circled fractions were selected for p24 and ddPCR analysis. These analyses confirmed presence of LV particles eluting in the void volume of the column, represented by the peak around the 19 minute mark. Remaining peaks between 25 and 45 minutes represent protein or nucleic acid sample impurities.

Initial inspection of the crude LV SEC profile revealed substantial sample heterogeneity, with minimal resolution of peaks between the 25 and 40 minute elution times. These peaks were considerably reduced, however, in the purified LV SEC profile. Given LV magnitude, in the mega-Dalton size range, it was expected that the LV particles would elute within the void volume of the column, in which large, unretained molecules elute, represented by the initial peak around the 19 minute mark. Indeed, the presence of LV particles eluting at this time was confirmed by p24 and droplet digital PCR (ddPCR) analysis of various elution fractions (F1-3, 6, & 9) of the purified LV injection (Table C).

TABLE C
p24 and ddPCR analysis results of selected purified LV elution fractions.
p24 ELISA Results

| | | | | | |
|---|---|---|---|---|---|
| Fraction | 1 | 2 | 3 | 6 | 9 |
| P24 (ng/mL) | <Range | ~5 | ~2 | <Range | <Range |

ddPCR Results

| | | | | | |
|---|---|---|---|---|---|
| Fraction | 1 | 2 | 3 | 6 | 9 |
| LV RNA | 2.60E+06 | 3.10E+07 | 4.10E+07 | 7.40E+06 | 3.60E+06 |
| (copies/mL) | | | | | |
| LV RNA per | 3.0% | 35.7% | 48.4% | 8.6% | 4.2% |
| Fraction | | | | | |

The p24 ELISA analysis of selected fractions revealed the highest concentration of p24 in the second fraction, corresponding with the void volume peak (FIG. 21 and Table C). In accordance with these results, ddPCR fraction analysis confirmed the highest concentration of LV RNA in fractions two and three (Table D), further supporting the elution of LV particles in the void volume peak. The remaining UV peaks between 25 and 45 minutes are thought to represent elution of residual protein and nucleic acid impurities. With identification of LV elution represented by the first peak eluting after ˜19 minutes, subsequent method development focused on enhancing resolution and separation of this peak from the remaining impurity peaks.

Various columns, flow rates, and buffers (summarized in Table B, Example 1) were evaluated in the interest of LV chromatography method optimization. Though column screening took place, the original TSKgel G5000PWXL column used in preliminary experiments was ultimately selected due to effective LV separation in the void volume from elution of other sample impurities. Additionally, increasing the flow rate beyond 0.300 mL/min was found to reduce the time between LV vector and impurity elution, minimizing peak separation (data not shown). As a result, the original 0.300 mL/min flow rate applied in preliminary experiments was also selected moving forward. Buffers were screened with the goal of maximizing LV vector stability while minimizing vector adsorption to the column. Finding this balance poses an important challenge in viral vector chromatography optimization. While LV sample additives may function to stabilize vector structure, they can also exert effects on vector-column hydrophobic interactions, thus influencing vector adsorption to the column.

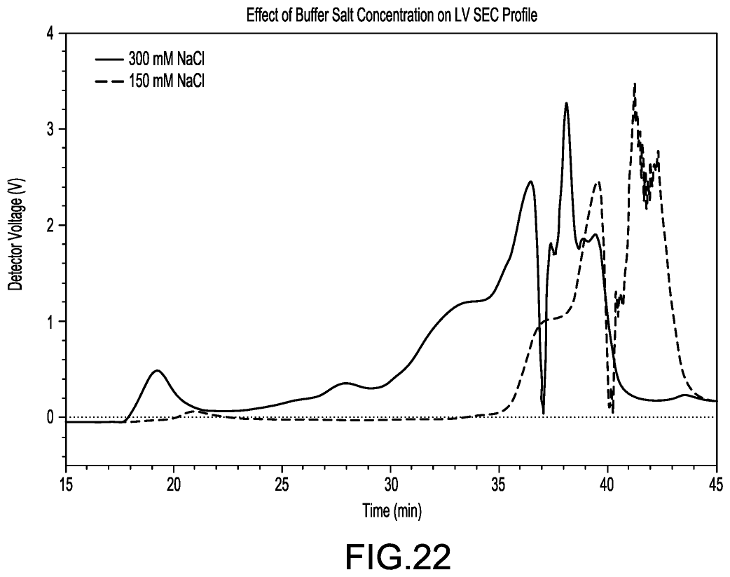

In the present study, NaCl, MgCl₂, and sucrose were evaluated as buffer components. Different pH values were not screened as pH 7.40, corresponding with cytosolic pH, was preferred. Given phosphate is prone to interacting with sample additives, such as metal ions, Tris was also evaluated as a buffer rather than the PBS used in preliminary experiments. This buffer screening process highlighted three notable observations. Firstly, addition of ethanol to the mobile phase, even minimally (˜10%), greatly reduced the LV UV peak signal (data not shown), suggesting LV vectors may be prone to degradation, aggregation, or precipitation in the presence of alcohol. Secondly, at least 300 mM NaCl was required to prevent LV vector adsorption to the column (FIG. 22), as evidenced by loss of LV UV peak signal and increased retention of sample peaks when NaCl concentration was lower than 300 mM. Thirdly, while MgC12 did not impact UV absorbance signals, the presence of sucrose in the mobile phase resulted in substantial adsorption and peak retention (data not shown). In light of these observations, 20 mM Tris buffer containing 300 mM NaCl and 2 mM MgCl2 at pH 7.40 applied at a 0.300 mL/min flow rate through the TSKgel G5000PWXL column were the parameters selected for all of the following LV chromatographic experiments.

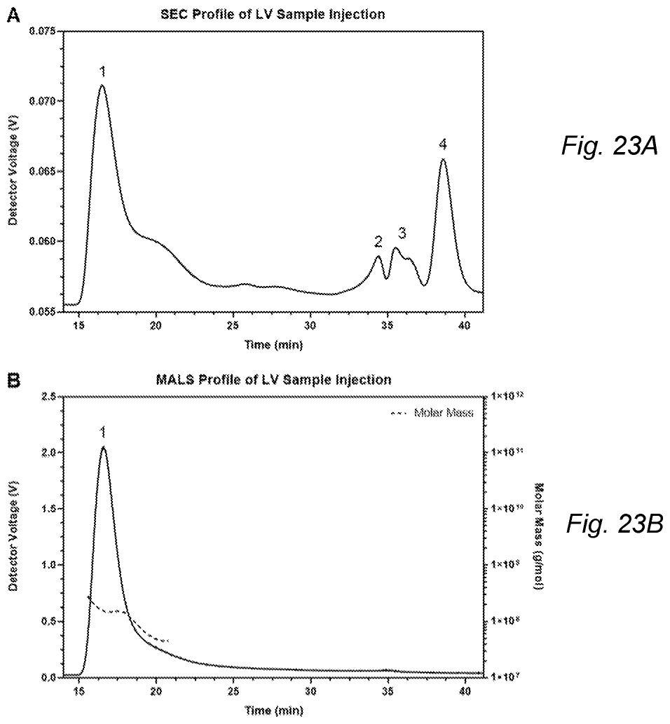

Developing an LV biophysical characterization method, the coupling of SEC to MALS was evaluated as a means of broadening the scope of accessible data. Following the previously outlined chromatographic parameters, 40 μL of purified LV was injected in triplicate on different days to obtain an average SEC-MALS profile (FIGS. 23A and 23B). While four distinguishable peaks were observed in the SEC UV profile (FIG. 23A), only one predominant peak was observed in the MALS elution profile which notably corresponded with the LV SEC elution peak (FIG. 23B).

LV particles are expected to substantially scatter light due to their large mega-dalton size. Because of this, the elution of LV particles will theoretically be represented by a large MALS peak (around the 17 minute mark). Indeed, the presence of such a peak corresponding with the void volume peak in the SEC elution profile further supports the previously reported p24 and ddPCR results indicating the elution of LV particles at this time. Additionally, the lack of MALS peaks corresponding with impurity peaks (peaks 2-4) in the SEC elution profile indicates these impurities are small protein or nucleic acid impurities which are not capable of scattering much light.

Molar mass analysis of the predominant MALS peak indicated the presence of higher-order species eluting with LV vector particles in the void volume of the column, as indicated by the drop in molar mass across the initial half of the MALS peak. This data corresponds with the p24 and ddPCR analysis which detected the majority of p24 and LV RNA copies in the second elution fraction represented by the latter half of the void volume peak (FIG. 21 and Table C). Importantly, initial quantitative analysis of the latter half of the predominant MALS peak revealed particles eluting at this time had an average molecular weight and radius consistent with literature values of LV particle size (Table D). With SEC, p24, ddPCR, and MALS analysis results all indicating elution of LV particles in the latter half of the void volume peak, subsequent method development focused on utilizing the MALS number density procedure to achieve titer estimations of LV particles eluting at this timeframe.

TABLE D.
Average size of LV particles detected by MALS
MALS Size Analysis Results of LV Particles

| Molecular Weight (Da) | 1.16E+08 ± 0.02 |
|---|---|
| Hydrodynamic Radius (nm) | 59.11 ± 1.33 |

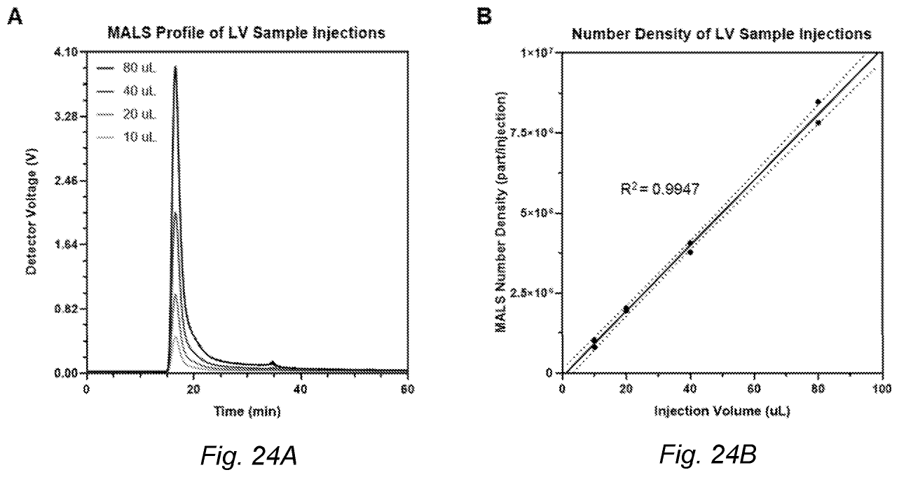

With the LV elution peak identified, quantification of LV particles by MALS was evaluated through a linearity study. LV sample volumes of 10 μL, 20 μL, 40 μL, and 80 μL were injected in triplicate on different days and evaluated using the MALS number density procedure. MALS peaks were proportional to injection volumes (FIG. 24A), and, consistent with initial MALS analysis and literature values, the average molecular weight and radius of LV particles eluted around 17 minutes was 1.27E+08±0.06 Da (n=10) and 62.50±3 nm (n=12), respectively. While the size of LV particles analyzed by the MALS detector remained consistent across injections, the number of particles detected increased linearly with sample volume (Table E). Confirmation of the linear model was achieved by F-test statistics with a 95% confidence interval between LV sample injections of 10 μL to 80 μL, with an average slope of 1.0×105, an average intercept of 1.1×10−5, and an average correlation coefficient of 0.9947 (FIG. 24B). Thus, moving forward, SEC-MALS was established as a useful biophysical tool for gaining both qualitative sample information, such as sample heterogeneity and molecular weight distribution, and quantitative information, i.e. diameter, molecular weight, and number density, of viral particles in solution. Notably, the MALS number density procedure, allowing direct quantification of viral particles without need of a calibration curve, was shown to be a new, reliable way of rapidly estimating viral particle titer.

TABLE E
Summary of LV size and titer estimation using the MALS number density procedure
MALS Number Density Analysis Results

| Injection Volume (uL) | 10 | 20 | 40 | 80 |
|---|---|---|---|---|
| Number Density (particles/injection) | 9.42E+05 ± 1.00 | 1.99E+06 ± 0.04 | 3.87E+06 ± 0.14 | 8.16E+06 ± 0.33 |
| Molecular Weight (Da) | 1.14E+08 ± 0.03 | 1.18E+08 ± 0.00 | 1.16E+08 ± 0.02 | 1.12E+08 ± 0.06 |
| Hydrodynamic Radius (nm) | 62.24 ± 1.46 | 61.50 ± 1.35 | 59.11 ± 1.33 | 57.11 ± 0.98 |

### Lentivirus pH Stability Investigation

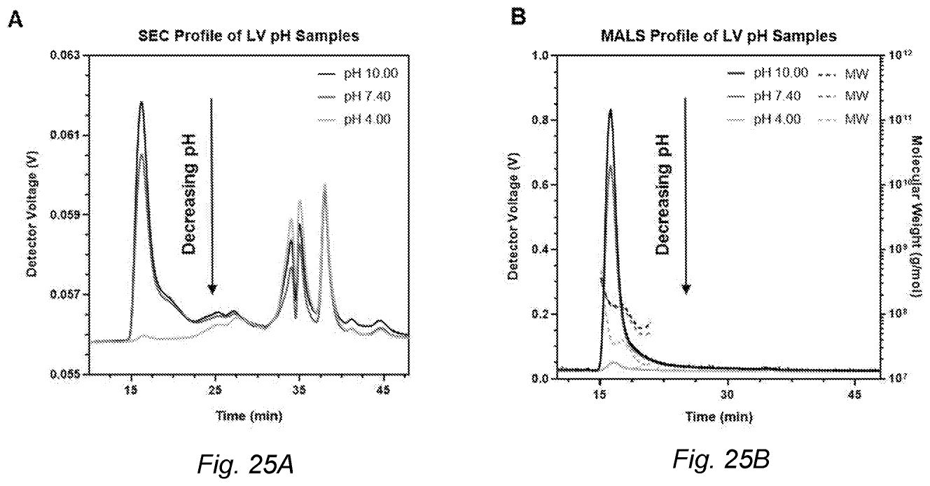

Having developed an SEC-MALS-based method demonstrated to be a valuable means of qualitatively and quantitatively assessing LV particles, it was applied to the subsequent LV pH stability investigation. LV samples were dialyzed to respective pH buffers directly prior to injection under the same conditions as the linearity study described above. The SEC elution profile of LVs at pH 4.00, 7.40, and 10.00 was monitored to encompass a wide pH range. As previously described, in-house ddPCR and p24 analysis confirmed the elution of LVs from this column ˜17 minutes following injection. Notably, the main LV 280 nm UV peak was almost completely lost in the SEC elution profile of LV particles at pH 4.00 (FIG. 25A). Furthermore, this peak was enlarged in the elution profile of LV particles at pH 10.00 compared to that at pH 7.00 (FIG. 25A). This same trend was mirrored in the MALS profiles (FIG. 25B), with the prominent LV MALS peak enlarged in the pH 10.00 profile and sharply decreased in the pH 4.00 profile. In accordance with these observations, MALS particle number and size analysis (Table F) revealed a ˜15 to ˜20 fold decrease in the number of particles at pH 4.00 compared to that at pH 7.40 and 10.00, respectively. Additionally, while particle hydrodynamic radius at pH 4.00 remained consistent, the molecular weight of pH 4.00 particles was ˜3 fold smaller than literature values (Table F). While the size of LV particles at pH 7.40 and 10.00 were consistent, the number of particles at pH 10.00 was increased ˜1 fold in accordance with the increased peak size observed in the pH 10.00 SEC-MALS profile (Table F, FIGS. 25A and 25B). These results suggested, not only vector disassembly at low pH, but perhaps preservation of vector integrity at high pH.

TABLE F
Summary of LV Size and Titer Estimation as a Function of pH
MALS Particle Number and Size Analysis Results

| pH | 4.00 | 7.40 | 10.00 |
|---|---|---|---|
| Number Density (particles/injection) | 7.91E+04 ± 0.29 | 1.21E+06 ± 0.23 | 1.55E+06 ± 0.21 |
| Molecular Weight (Da) | 3.55E+07 ± 0.04 | 1.26E+08 ± 0.02 | 1.34E+08 ± 0.02 |
| Hydrodynamic Radius (nm) | 62.71 ± 1.60 | 62.94 ± 0.76 | 62.13 ± 0.78 |

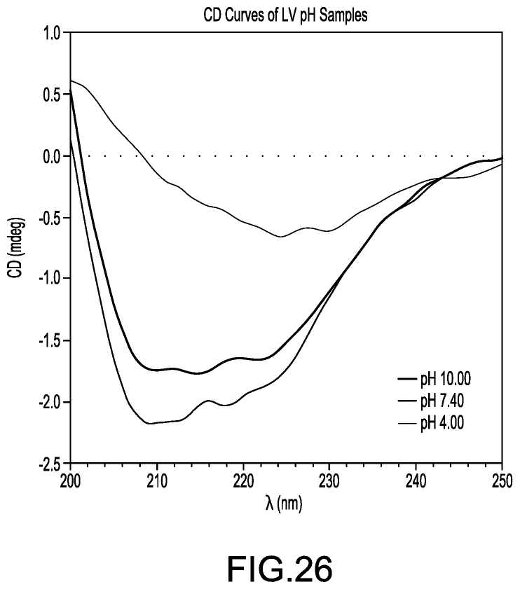

In order to comprehensively evaluate the effect of pH on LV particle disassembly, circular dichroism (CD) was used to determine the effect of pH on LV secondary structure. CD spectra result from the cumulative interaction of light with chiral molecules. Each protein has a specific CD signature that is largely influence by the overall 3D structure of the protein. This makes CD a useful tool for analyzing protein secondary structure. Furthermore, protein secondary-structural elements such as alpha helices and beta-sheets have characteristic CD signals. A predominantly alpha-helical structure is characterized by two minima around 210 nm and 220 nm, while a structure that predominantly consists of beta-sheets is characterized by one minimum around 220 nm. CD data for LV particles at pH 7.40 suggests a predominantly alpha-helical conformation (FIG. 26). Consistent with SEC-MALS analysis of the LV pH samples, this alpha-helical conformation is entirely lost in LV particles at pH 4.00, suggesting complete vector disassembly. However, vector protein conformation is retained in LV particles at pH 10.00, supporting the notion that high pH serves to protect LV vector integrity.

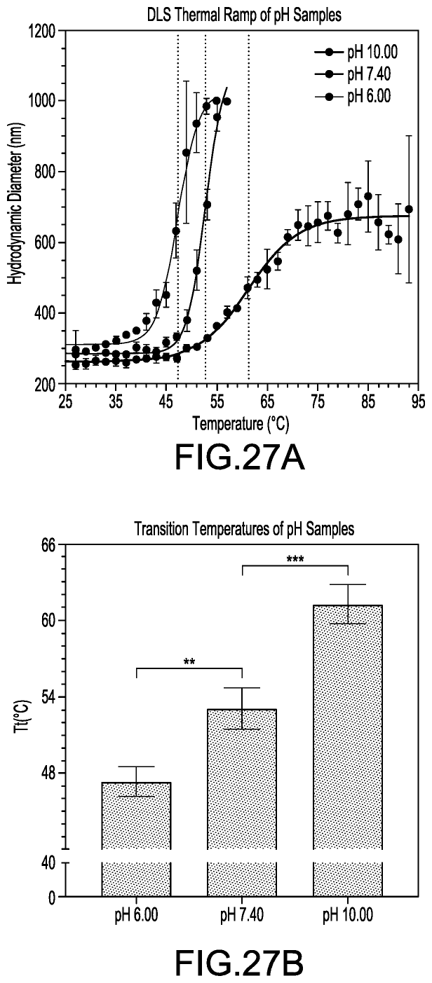

With consistent results from both SEC-MALS and CD data suggesting a positive correlation between pH and LV particle integrity, the thermal stability of LV particles was evaluated as a function of pH by monitoring dynamic light scattering (DLS) of LV particles with increasing temperature. DLS measures the rate at which particles suspended in a solution diffuse. These particles undergo Brownian motion which dictates that larger particles will diffuse more slowly than smaller ones. DLS approximates a particle's size based on the rate at which it diffuses. As the unfolding of proteins is linked to their aggregation, a DLS melting curve can be used to determine the melting point of LV particles. In other words, as the proteins of the LV particles unfold, the particles will aggregate and collectively scatter light, which is perceived by the DLS instrument as one large particle. Thus, sharp increases in particle size indicate protein unfolding with temperature. Of note, particles dialyzed to pH 4.00 directly preceding DLS assessment were already aggregated beyond the DLS instrument's limit of detection (>1000 nm diameter). For this reason, the DLS melting curve of LV particles dialyzed to pH 6.00, 7.40, and 10.00 is reported (FIGS. 27A and 27B). These results indicated LV particles at pH 6.00 were least thermally stable, with a melting temperature of ˜47° C., followed by those at pH 7.40 with a melting temperature of ˜53° C., and, finally, pH 10.00 at ˜61° C. (FIG. 27B). Interestingly, while LV particles at pH 6.00 and 7.40 quickly reached the DLS instrument's upper limit of detection upon melting, LV particles at pH 10.00 never reached this limit (FIG. 27A). Thus, as evaluated by SEC-MALS, CD, and DLS, the integrity of LV particles appears to be directly correlated with pH as results obtained from all three techniques suggest vector disassembly at low pH and vector preservation at high pH.

### Lentivirus Salt Stability Investigation

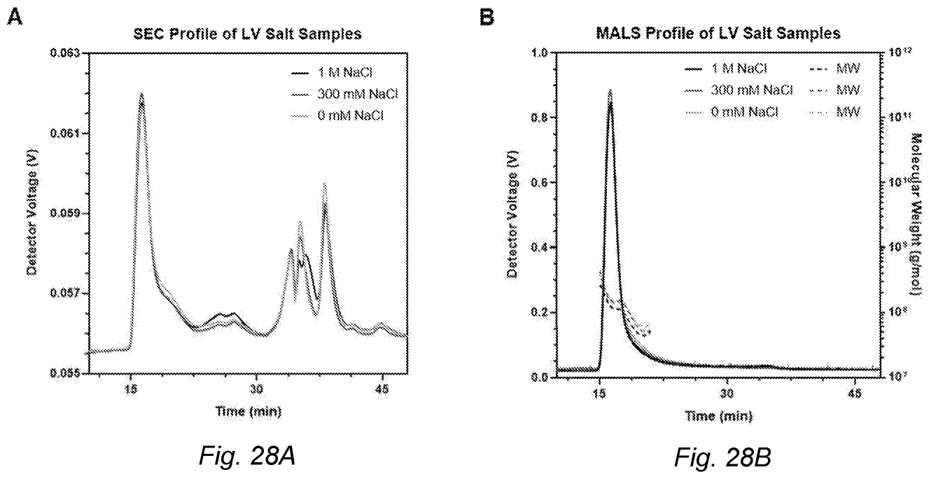

In addition to pH, LV particle stability was evaluated as a function of pH using the same SEC-MALS based method and biophysical tools. LV samples were dialyzed to respective salt buffers directly prior to injection under the same chromatographic conditions as the linearity and pH studies described above. The SEC elution profile of LVs at 0 mM, 300 mM, and 1 M NaCl was monitored. Interestingly, unlike in the pH stability study, no major differences were observed in the LV sample SEC-MALS profile as a function of salt concentration (FIGS. 28A and 21B). Similarly, the molecular-weight trends across the predominant MALS peaks were consistent across ionic conditions (FIG. 28B). Additionally, though the size of LV particles determined by MALS at all three ionic conditions was consistent with literature values, the number of LV particles increased slightly with increasing salt concentration.

TABLE G
Summary of LV Size and Titer Estimation as a Function of Salt
MALS Particle Number and Size Analysis Results

| Salt Concentration | 0 mM | 300 mM | 1 mM |
|---|---|---|---|
| Number Density (particles/injection) | 1.46E+06 ± 0.24 | 1.54E+06 ± 0.22 | 1.79E+06 ± 0.22 |
| Molecular Weight (Da) | 1.40E+08 ± 0.03 | 1.44E+08 ± 0.02 | 1.11E+08 ± 0.03 |
| Hydrodynamic Radius (nm) | 63.27 ± 0.77 | 62.34 ± 0.77 | 60.78 ± 0.75 |

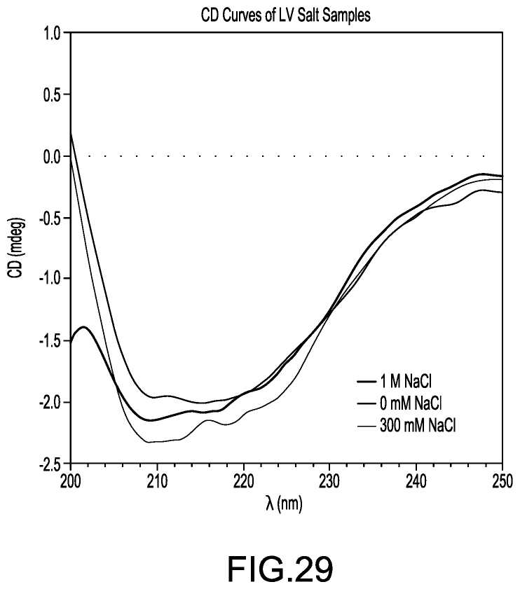

Though no effects of salt concentration on LV particle disassembly were apparent using SEC-MALS, CD was used to see if LV secondary structure altered with ionic strength. Consistent with SEC-MALS results, a predominantly alpha-helical structure was observed for LV particles at all three ionic conditions (FIG. 29), further suggesting salt concentration does not have any immediate effects on LV particle integrity.

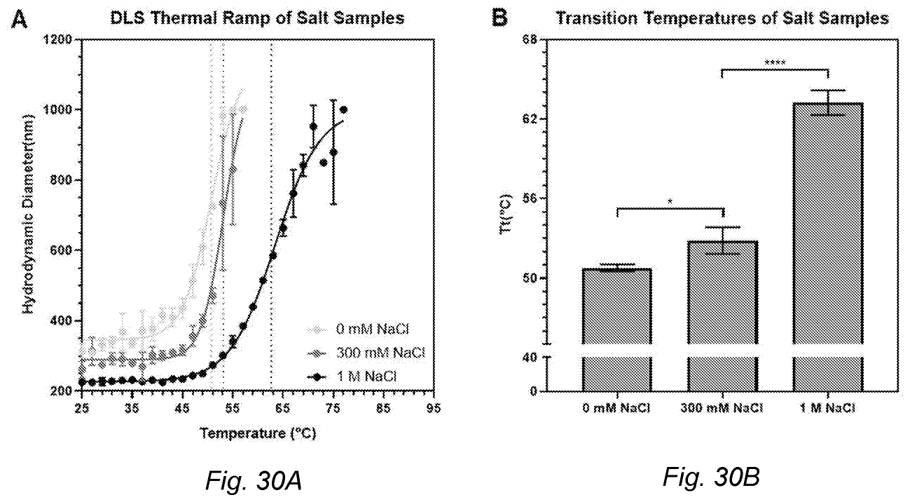

Though ionic conditions did not have any immediate effects on LV particle integrity as observed by SEC-MALS and CD, their effect on LV particle thermal stability was evaluated using a DLS thermal ramp. Interestingly, the thermal stability of LV particles was found to increase with salt concentration. The DLS melting curve of LV particles dialyzed to 0 mM, 300 mM, and 1 M NaCl is reported (FIG. 30A). These results indicated LV particles at 0 mM NaCl were least thermally stable, with a melting temperature of ˜50° C., followed by those at 300 mM NaCl with a melting temperature of ˜53° C., and, finally, 1 M NaCl at ˜63° C. (FIG. 30B). Thus, LV particles appear less stable at low ionic conditions, with high salt concentration functioning to increase LV particle stability while decreasing aggregation propensity.

Adeno-associated Virus type 5 Thermal Integrity Investigation

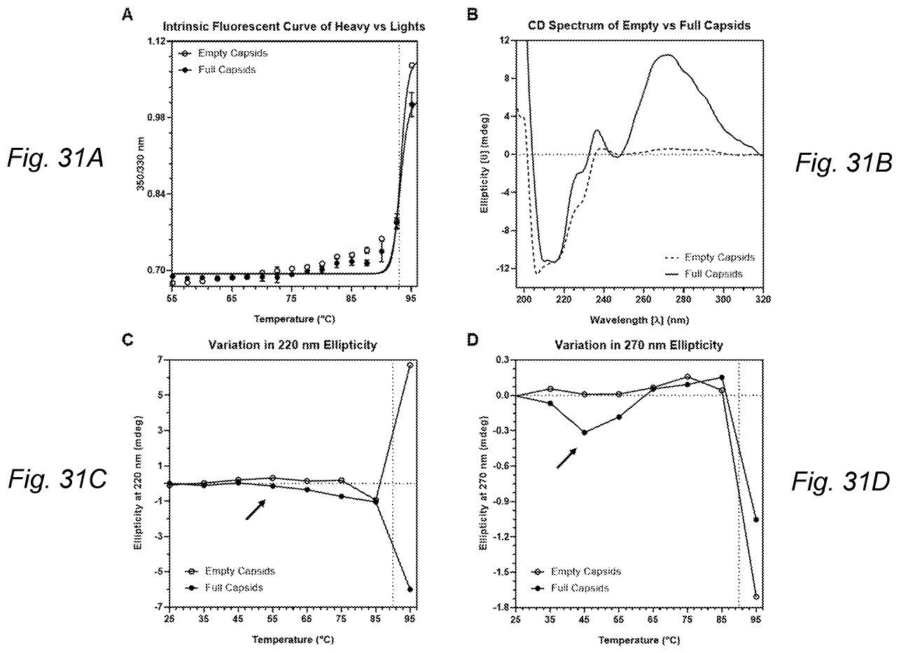

Differential scanning fluorimetry (DSF) was used to investigate the role of encapsulated genome size on AAV capsid thermal stability. In this technique, capsids are heated in the presence of a fluorescent dye (often SYPRO-orange™) which binds to hydrophobic regions of unfolding proteins. As the capsid proteins unfold, their hydrophobic pockets are exposed, enabling binding of the dye and greater fluorescence. Hence, increases in fluorescence indicate the unfolding of proteins with temperature. Using this technique, the melting temperature of AAV5 capsids has been reported as ˜89° C., regardless of encapsulated genome size. Indeed, using an analogous method, the melting temperature of empty (no encapsulated genome) and full (encapsulated genome) rAAV5 capsids was consistent with these reports (FIG. 31A). These results were obtained measuring the intrinsic fluorescence of tryptophan and tyrosine residues of the capsid proteins. As the proteins unfold, their tryptophan and tyrosine residues gain exposure to solvent, which increases their fluorescence. Thus, similarly to DSF, increases in intrinsic fluorescence signify protein unfolding with temperature. While these initial results appeared to support the insignificance of genome size in determining capsid thermal stability, results supporting the contrary were achieved shortly thereafter using CD. Initial representative far-UV CD spectra of empty and full capsids showed differences at 210 nm and 270 nm (FIG. 31B). Empty-capsid CD spectra displayed a curve suggesting a predominantly alpha-helical conformation, with a prominent 210 nm minimum, compared to full capsids, which had a curve with the 220 nm minimum more indicative of beta-sheets. Not surprisingly, full-capsid CD spectra showed absorption at 270 nm, where DNA absorbs light, which was not observed for empty-capsids. Of note, while CD-obtained melting temperatures for both empty and full capsids were consistent with previous reports, a biphasic melting curve was observed for full capsids that was not seen for empty ones (FIGS. 31C and 31D). This biphasic curve was observed when monitoring CD ellipticity at 220 nm (FIG. 31C) and 270 nm (FIG. 31D). These results warranted further investigation of the thermal integrity of full capsids in comparison to light ones.

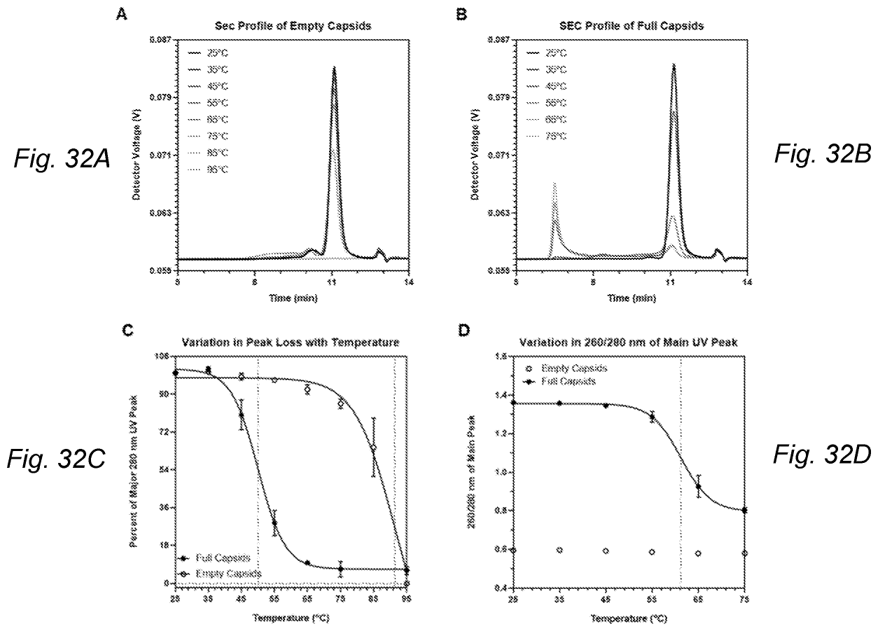

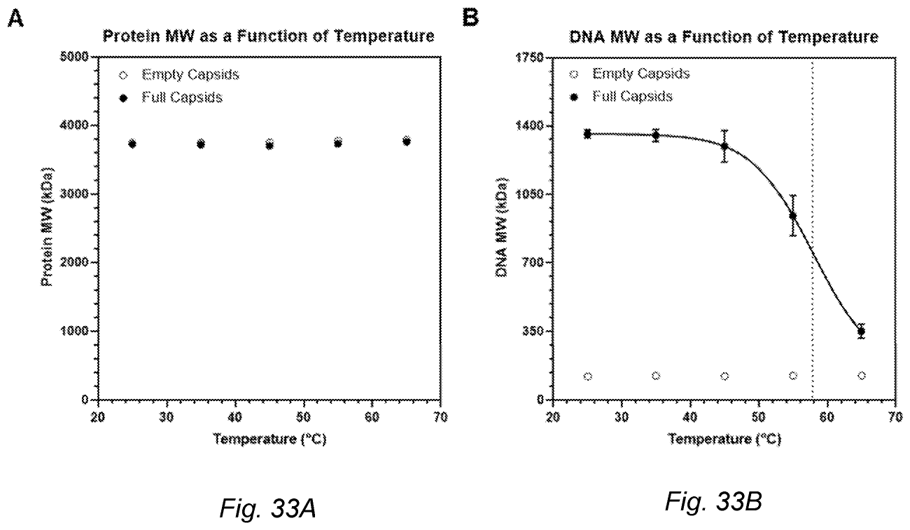

To ascertain any differences between the integrity of empty and full capsids with heat exposure, SEC-MALS was used to evaluate both types of capsids incubated at 10° C. intervals ranging from 25° C.-95° C. Capsid samples were incubated for 30 minutes at each respective temperature directly prior to injection with 2× PBS+10% EtOH mobile phase at a 1.0 mL/min flow rate. Following incubation at 25° C., both capsid types displayed a uniform SEC profile, with a predominant 280 nm UV peak (FIGS. 32A and 32B) and corresponding MALS peak (not shown) around 11 minutes after injection. While this profile remained unchanged for both empty and full capsids after incubation at 35° C., two very distinct trends were observed with increasing heat in the injections that followed (FIGS. 32A, 32B, and 32C). For the empty capsids, a gradual ˜15% reduction of the predominant capsid peak was observed from 45° C. to 75° C. (FIGS. 32A and 32C). After incubation at 85° C. the percentage of the main peak dropped to —65% of the original and then plummeted to nearly 0% following incubation at 95° C. (FIGS. 32A and 32C). In stark contrast, the main peak for the full capsids dropped ˜20% after incubation at 45° C. and then plummeted to ˜10% of the original by 65° C. (FIGS. 32B and 32C). The trends observed in both empty and full capsid UV profiles was mirrored by the MALS elution profiles (data not shown). Because nucleic acids absorb light at 260 nm while proteins absorb at 280 nm, comparing the area under the curve of the 260 nm vs 280 nm UV elution profiles to obtain a 260:280 ratio can be a useful tool in characterizing the protein-DNA capsid complex. Monitoring this ratio as a function of temperature highlighted an interesting transition in the full capsids which was absent in the empty ones. As expected, the initial 260:280 ratio of full capsids was much higher than light ones at 25° C. (˜1.4 vs ˜0.6), due to their encapsulated genome (FIG. 32D). However, while the 260:280 ratio of empty capsids remained constant up to 75° C., a drop in this ratio was observed for full capsids around 60° C. (FIG. 32D). These results indicated that the amount of encapsulated DNA in full capsids decreased at this temperature, suggesting possibly a breakdown in capsid structure and subsequent DNA leakage. To explore this further, the MALS protein-conjugate procedure was used to monitor the molecular weight of the protein and encapsulated DNA separately as a function of temperature for both empty and full capsids. This analysis confirmed that while the molecular weight of the protein capsid remains constant from 25° C. to 65° C. for both empty and full capsids (FIG. 33A), the molecular weight of the encapsulated DNA in heavy capsids decreases as a function of temperature (FIG. 33B). These results supported the notion that capsids containing a genome breakdown and spill their encapsulated DNA at a much earlier temperature than 1) empty capsids breakdown and 2) melting of capsid proteins. Importantly, the trend observed in the thermal stability of full capsids as assessed by SEC-MALS was consistent with the biphasic event observed in the CD melting curve, indicating the presence of a transition temperature at which capsid breakdown occurs long before melting of the capsid proteins around 90° C.

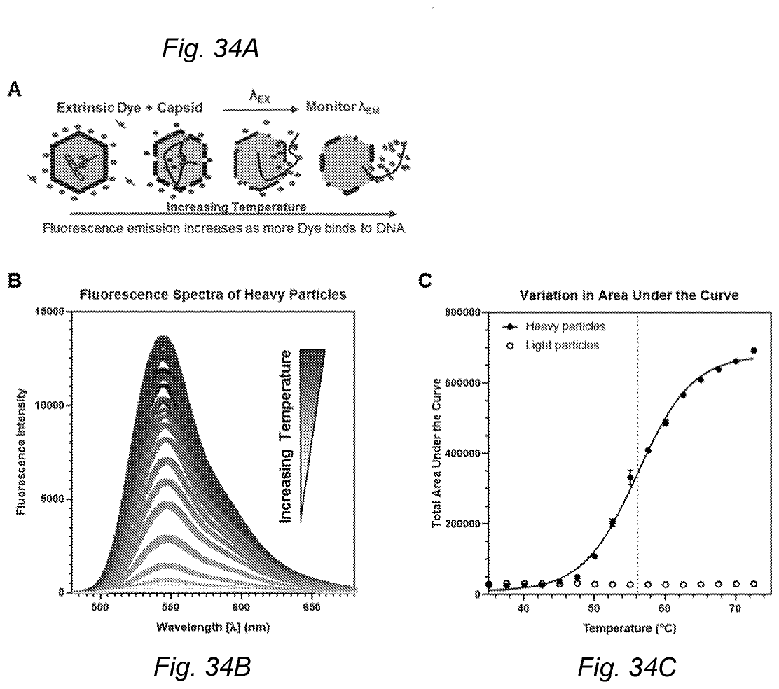

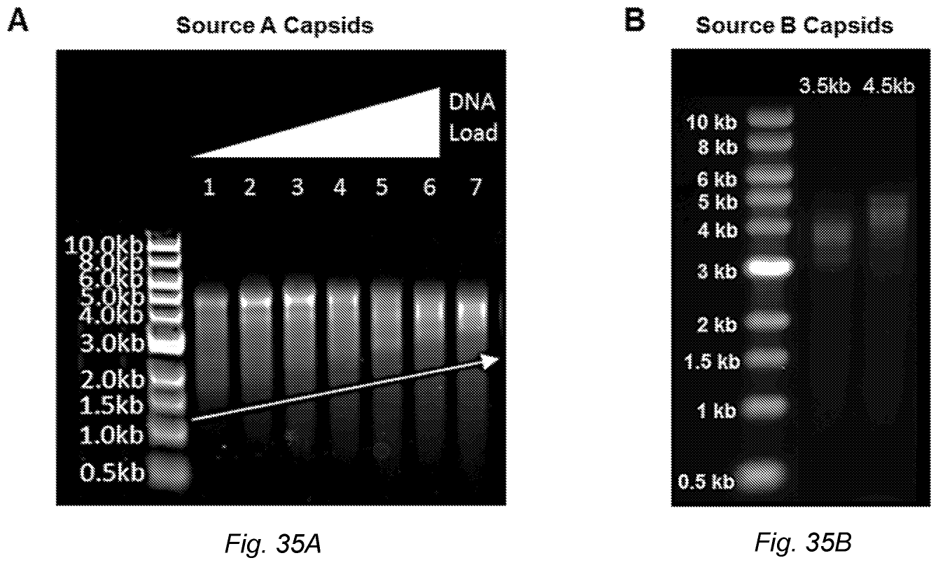

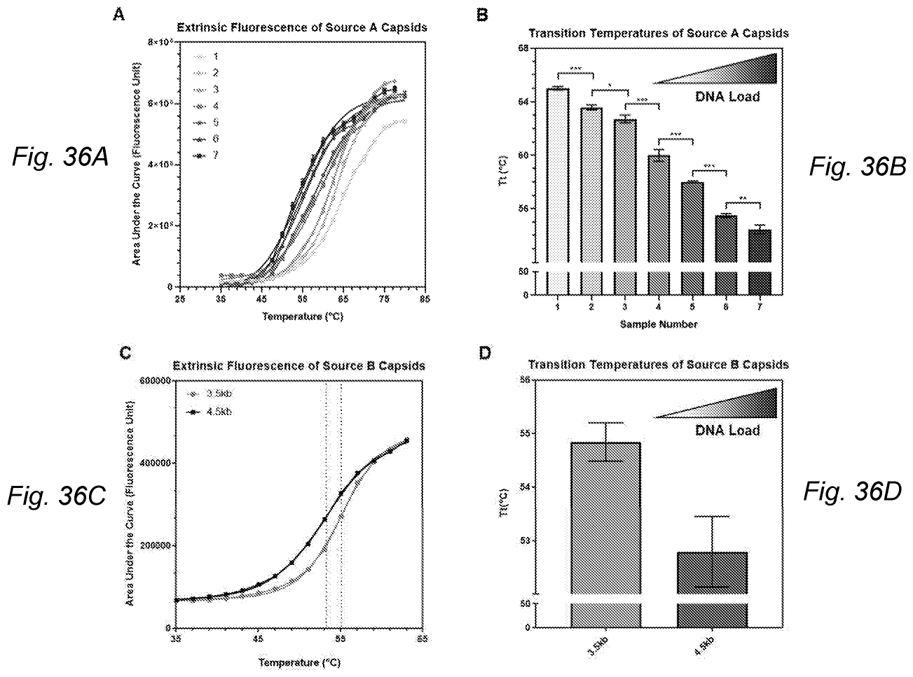

With evidence to support the notion that presence of an encapsulated genome affects capsid stability, rAAV5 capsids coating a range of single-stranded genome sizes were assessed to more closely monitor the effect of genome size on thermal integrity. If capsid integrity is indeed compromised before the melting of capsid proteins at 90° C., DNA should be released into solution. Therefore, to monitor capsid disassembly, extrinsic capsid fluorescence was monitored using SYBR Gold Nucleic Acid dye. The premise behind this method involves the binding of SYBR Gold dye to DNA expelled from capsids into solution (FIG. 34A). As the capsids break down, more DNA is able to bind SYBR Gold, and the intensity of the fluorescent curve increases. Thus, the extrinsic fluorescent curve can be used to monitor the breakdown of capsids rather than capsid melting temperature (as evaluated by DSF and intrinsic fluorescence). To test this method, the fluorescence of both full and empty rAAV5 capsids were mixed with SYBR gold dye was measured from 25° C. to 95° C. As expected, the fluorescent curve of full capsids increased with temperature (FIG. 34B), indicating binding of SYBR gold dye to expelled DNA, while the fluorescent curve of empty capsids was negligible due to the lack of DNA for SYBR gold to bind (data not shown). By plotting the area under the fluorescent curves as a function of temperature, a distinct trend and transition temperature was observed for heavy capsids, consistent with the trend observed in the CD thermal melts (FIG. 34C). No trend was observed for empty capsids (FIG. 34C). These results further indicate the presence of a transition temperature upon which capsid breakdown occurs —55° C. Using this method, rAAV5 capsids coating a range of single-stranded genome sizes were assessed to determine any perceivable difference in the transition temperature of capsids as a function of encapsulated genome size. Extrinsic fluorescence of capsid samples 1-7, coating a range of single stranded genomes in order of increasing genome size (FIG. 35A), mixed with SYBR Gold dye was evaluated as a function of temperature. Strikingly, capsid transition temperatures inversely correlated with genome size (FIGS. 36A and 36B). That is, a linear trend in transition temperatures was observed among capsid samples 1-7, with capsids in sample 1 having the highest transition temperature and capsids in sample 7 having the lowest. These results indicate that capsids coating larger genomes are less thermally stable than those coating smaller genomes.

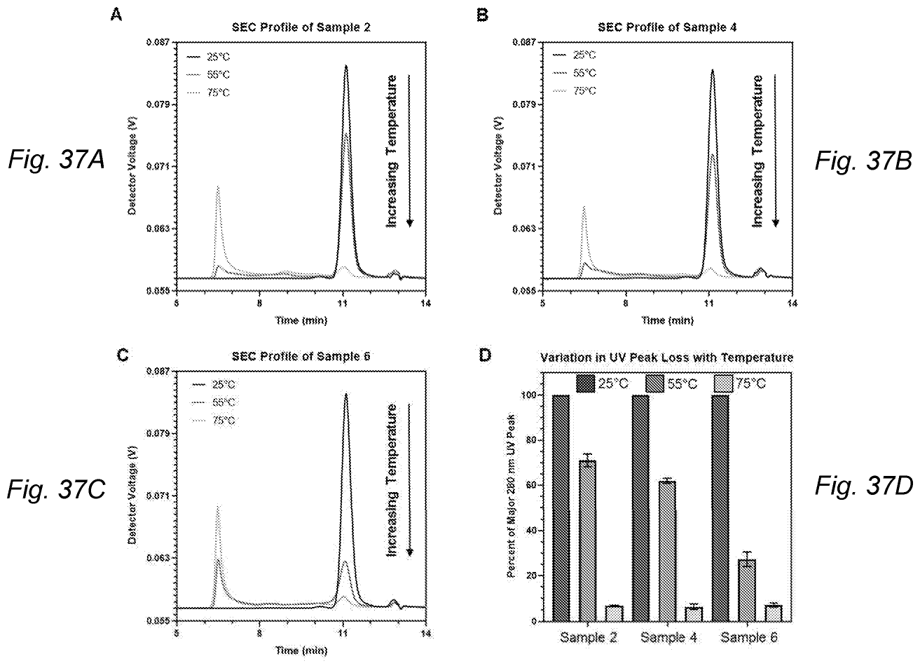

To further evaluate the effect of genome size on rAA5 capsid integrity, the thermal stability of capsids from samples 2, 4, and 6 (encompassing a wide range of genome size) were evaluated via SEC-MALS. Capsid samples were incubated for 30 minutes at 25° C., 55° C., and 75° C. directly prior to injection onto an SEC-1000 Sepax column with 2× PBS+10% EtOH mobile phase at a 1.0 mL/min flow rate. Following incubation at 25° C., all capsids displayed a uniform SEC profile, with a predominant 280 nm UV peak (FIGS. 37A, 37B, and 37C) and corresponding MALS peak (not shown) around 11 minutes after injection. However, differences in the area of the predominant UV peak were observed following incubation at 55° C. between each of the three capsid samples (FIGS. 37A, 37B, and 37C). While a ˜29% reduction in the main UV peak was observed for sample 2 capsids (coating the smallest genome), a ˜38% reduction was observed for sample 4 capsids, and a large ˜73% reduction was observed for sample 6 capsids (coating the largest genome) (FIG. 37D). Following incubation at 75° C., the main UV peak of all three capsids dropped to less than ˜10% of the original. While these results indicate that all three capsid structures broke down before 75° C., the onset of capsid breakdown was different for all three as regulated by the size of their encapsulated genome.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

1. A method for characterizing and quantifying viral particles comprising the steps of:
analyzing by size exclusion chromatography (SEC) a sample containing viral particles having vector genomes encapsulated within capsids;
and from the SEC analysis, determining at least one of the following:
quantifying aggregation of the viral particles in the sample,
quantifying a concentration of nucleic acid and/or protein impurities in the sample,
determining weight averaged molecular weights of the capsids and/or vector genomes,
and quantifying concentrations of viral particles and/or capsids devoid of encapsulated vector genomes in the sample.

2. The method of claim 1, wherein the SEC analysis comprises fractioning the sample by size exclusion chromatography and measuring light absorption by the fractions at wavelengths of about 260 nanometer (nm) and about 280 nm.

3. The method of claim 2, further comprising identifying viral particles in at least one of the fractions when the at least one of the fractions exhibits a first wavelength to the second wavelength (first wavelength: second wavelength) absorbance ratio of 1.13 to less than 1.75.

4. A method for monitoring the structural integrity of capsids comprising the steps of:
analyzing by SEC a plurality of samples from a preparation comprising viral particles having vector genomes encapsulated within capsids, where each of the samples is modified to have a property that is different from the others; and
from the SEC analysis, monitoring the structural integrity of the capsids in each of the samples;
wherein changes in protein and nucleic acid concentrations between the samples correlate to changes in the structural integrity of the capsids.

5. The method of claim 4, wherein the SEC analysis comprises fractioning the sample by size exclusion chromatography and measuring light absorption by the fractions at wavelengths of about 260 nm and about 280 nm.

6. The method of claim 5, further comprising identifying viral particles in at least one of the fractions when the at least one of the fractions exhibits a first wavelength to the second wavelength (first wavelength: second wavelength) absorbance ratio of 1.13 to less than 1.75.

7. The method of claim 4, which comprises determining storage stability of the samples of lengths of time at 25° C. based on the structural integrity of the capsids.

8. A method for characterizing and/or quantifying viral particles comprising the steps of:
analyzing by SEC and size exclusion chromatography multi-angle lights scattering (SEC-MALS) a sample of a preparation of viral particles having vector genomes encapsulated within capsids;
and from the SEC and SEC-MALS analysis determining at least one of the following,
quantifying aggregation of the viral particles in the preparation,
quantifying a concentration of nucleic acid and/or protein impurities in the preparation,
determining weight averaged molecular weights of the capsids and vector genomes, and/or
quantifying concentrations of viral particles and capsids devoid of encapsulated vector genomes in the preparation.

9. The method of claim 8, wherein the SEC analysis comprises fractioning the sample by size exclusion chromatography and measuring light absorption by the fractions at wavelengths of about 260 nm and about 280 nm.

10. The method of claim 9, further comprising identifying intact rAAV particles in at least one of the fractions when the at least one of the fractions exhibits a first wavelength to the second wavelength (first wavelength: second wavelength) absorbance ratio of 1.13 to less than 1.75.

11. The method of claim 8, wherein the SEC-MALS analysis comprises fractioning the sample by size exclusion chromatography and measuring at least one of refractive indexes of the fractions, light absorption by the fractions at a wavelength within the ultraviolet spectrum, and intensity of light scatter by the fractions using multiangle light scattering analysis (MALS).

12. The method of claim 8 further comprising determining a size distribution of the viral particles and/or the capsids devoid of encapsulated vector genomes in the preparation by dynamic light scattering analysis.

13. The method of claim 12, wherein the size distribution of the viral particles is the radius of gyration (Rg) and/or hydrodynamic radius (Rh) of the viral particles.

14. The method of claim 8, wherein the quantifying concentrations of viral particles and capsids devoid of encapsulated vector genomes in the preparation comprises determining the Rg and Rh of the viral particles and/or the capsids devoid of encapsulated vector genomes by dynamic light scattering analysis, wherein a ratio of Rg to Rh correlates to the percentage concentration of viral particles in the preparation.

15. A method for monitoring the structural integrity of capsids comprising the steps of:
analyzing by SEC and SEC-MALS a plurality of samples from a preparation comprising viral particles having vector genomes encapsulated within capsids, where each of the samples is modified to have a property that is different from the others; and
from the SEC and SEC-MALS analysis, monitoring the structural integrity of the capsids in each of the samples;
wherein changes in protein and nucleic acid concentrations between the samples correlate to changes in the structural integrity of the capsids.

16. The method of claim 15, wherein the SEC analysis comprises fractioning the sample by size exclusion chromatography and measuring light absorption by the fractions at wavelengths of about 260 nm and about 280 nm.

17. The method of claim 16, further comprising identifying intact rAAV particles in at least one of the fractions when the at least one of the fractions exhibits a first wavelength to the second wavelength (first wavelength: second wavelength) absorbance ratio of 1.13 to less than 1.75.

18. The method of claim 15, wherein the SEC-MALS analysis comprises at least one of fractioning the sample by size exclusion chromatography and measuring refractive indexes of the fractions, light absorption by the fractions at a wavelength within the ultraviolet spectrum, and intensity of light scatter by the fractions using MALS.

19. The method of claim 15, which comprises determining storage stability of the samples of lengths of time at 25° C. based on the structural integrity of the capsids.

20. The method as claim 8, wherein the quantifying and determining steps do not require a calibration curve.

21. The method as claim 1, wherein the viral particles are adeno-associated viral particles or lentivirus viral particles.

22. The method as in claim 1 further comprising, prior to SEC or SEC-MALS analysis, analyzing the sample or plurality of samples by analytical ultracentrifugation to identify the viral particles and/or capsids without encapsidated vector genomes.
